SEQUENCE LISTING (18 sequences)

SEQ ID NO: 1 - protein, 674 aa, Enterococcus faecalis
MTKSVKFLVLLLVMILPIAGALLIGPISFGAELSKSSIVDKVELDHTTLYQGEMTSIKVSFSDKENQKIKPGDTITLTLPDALVGMTENDGSPRKINLNGLGEVFIYKDHVVATFNEKVESLHNVNGHFSFGIKTLITNSSQPNVIETDFGTATATQRLTIEGVTNTETGQIERDYPFFYKVGDLAGESNQVRWFLNVNLNKSDVTEDISIADRQGSGQQLNKESFTFDIVNDKETKYISLAEFEQQGYGKIDFVTDNDFNLRFYRNKARFTSFIVRYTSTITEAGQHQATFENSYDINYQLNNQDATNEKNTSQVKNVFVEGEASGNQNVEMPTEESLDIPLETIDEWEPKTPTSEQATETSEKTGATETAESSQPEVHVSPTEEENPDEGGTLGTIEPIIPEKPSVTTEENGVTETAEGSQPEVHVSPTEEENPDESETLGTITPIIPEKPSVTTEENGATETAESSQPEVHVSPAEEENPDESETLGTITPIIPEKPSVTTEENGTTETAESSQPEVHVSPTEEENLDESETLGTIEPIIPEKPSVTTEENGTTETAESSQPEVHVSPTKEITTTEKKQPSTETTVEKNKNVTSKNQPQILNAPLNTVKNEGSPQLAPQLLSEPIQKLNEANGQRELPKTGTTKTPFMLIAGILASTFAVLGVSYLQIRKN

SEQ ID NO: 2 - protein, 317 aa, Enterococcus faecalis
MTKIYRRLIIGVTLAISAFLLASCGQTTQSPKEKKELTVMTTFYPMYDFAKQVVGDEGEVELLIPAGTEPHDYEPSAKDLAKITDADVFVYNSKELETWVPNVIENLDTKKVSIVEASQSIQLMQGTEEEESGEEGHEGHNHSHELDPHVWLDPVLAQKEVTAIRDALIKKYPEKKAVFEKNTAAYLEKLTALDKEYQAAFAGAKNRTFVTQHAAFGYLAKQYGLTQEPIAGISPDQEPSPSRLAELKKYIKTNNVSVIYFEASASTKVAKTLADETGVELAVLNPLESLTQKEQEAGENYVSVMKENLAALQKSIH

SEQ ID NO: 3 - protein, 511 aa, Enterococcus faecalis
MKKFTLPLLAALSLILFGACGKTNTSDKTADGKEKLSIVTTFYPMYDFTKNIVGDEGDVKLLIPAGSEPHDYEPSAKDMATIHDADVFVYHNENMESWVPKAAKGWKKGAPNVIKGTENMVLLPGSDEDGHDHDHEHGEEGHHHELDPHTWVSPHRAIQEVTNIKEQLVKLYPKKAKTFETNAEKYLTKLTALDKEFQTALKDAKQKSFVTQHAAFGYLALDYGLKQVPIAGLTPEQEPTAGRLAELKKYVTDNQIRYIYFEKNANDKIAKTLADEANVQLEVLNPLESLTQKQMDNGEDYLSVMKENLTALKKTTDTAGKEVQPETSEKTEKTVANGYFKDSEVAERTLADYAGNWQSVYPLLKDGTLDQVFDYKAKLKKDKTPAEYKTYYDAGYQTDVDHINITDSTIEFLVDGKPQKFTYKAAGYKILNYAKGNRGVRFLFETDDANAGRFKYVQFSDHNIAPTKAAHFHIFFGGDSQESLFNEMDNWPTYYPNDLSKQEIAQEMIAH

SEQ ID NO: 4 - protein, 1154 aa, Enterococcus faecalis
MLIHKRKERTDIKLLKKFGVFGVVFLLVSSYFIPLIGYAETAKEVEITSAQMITDENDKTNINIELNLLNQTEQPLQREIQLKNAQFMDTAVIEKDGYSYQVTNGTLYLTLDAQVKKPVQLSLAVEQSSLQTAQPPKLLYENNEYDVSVTSEKITVEDSAKESTEPEKITVPENTKETNKNDSAPENTEQPTATEEVTNPFAEARMAPATLRANLALPLIAPQYTTDNSGTYPTANWQPTGNQNVLNHQGNKDGGTQWDGQTSWNGDPTNRTNSYIEYGGTGDQADYAIRKYARETTTPGLFDVYLNVRGNVQKEITPLDLVLVVDWSGSMNENNRIGEVQKGVNRFVDTLADSGITNNINMGYVGYSSDSYNNNAIQMGPFDTVKNPIKNITPSSTRGGTFTQKALRDAGDMLATPNGHKKVIVLLTDGVPTFSYKVSRVQTEADGRFYGTQFTNRQDQPGSTSYISGSYNAPDQNNINKRINSTFIATIGEAMALKQRGIEIHGLGIQLQSDPRANLSKQQVEDKMREMVSADENGDLYYESADYAPDISDYLAKKAVQISGTVVNGKVVDPIAEPFKYEPNTLSMKSVGPVQVQTLPEVSLTGATINSNEIYLGKGQEIQIHYQVRIQTESENFKPDFWYQMNGRTTFQPLATAHEKVDFGVPSGKAPGVKLNVKKIWEEYDQDPTSRPDNVIYEISRKQVTDTANWQTGYIKLSKPENDTSNSWERKNVTQLSKTADESYQEVLGLPQYNNQGQAFNYQTTRELAVPGYSQEKIDDTTWKNTKQFKPLDLKVIKNSSSGEKNLVGAVFELSGKNVQTTLVDNKDGSYSLPKDVRLQKGERYTLTEVKAPAGHELGKKTTWQIEVSEQGKVSIDGQEVTTTNQVIPLEIENKFSSLPIRIRKYTMQNGKQVNLAEATFALQRKNAQGSYQTVATQKTDTAGLSYFKISEPGEYRMVEQAGPLGYDTLAGNYEFTVDKYGKIHYAGKNIEENAPEWTLTHQNHLKPFDLTVHKKADNQTPLKGAKFRLTGPDTDIELPKDGKETDTFVFENLKPGKYVLTETFTPEGYQGLKEPIELIIREDGSVTIDGEKVADVLISGEKNNQITLDVTNQAKVPLPETGGIGRLWFYLIAISTFVIAGVYLFIRRPEGSV

SEQ ID NO: 5 - protein, 308 aa, Enterococcus faecalis
MKKFSLFFLTLLAGLTLAACGNQAAEKKEKLAIVTTNSILSDLVKNVGQDKIELHSIVPIGTDPHEYEPLPEDIAKASEADILFFNGLNLETGGNGWFNKLMKTAKKVENKDYFSTSKNVTPQYLTSAGQEQTEDPHAWLDIENGIKYVENIRDVLVEKDPKNKDFYTENAKNYTEKLSKLHEEAKAKFADIPDDKKLLVTSEGAFKYFSKAYDLNAAYIWEINTESQGTPEQMTTIIDTIKKSKAPVLFVETSVDKRSMERVSKEVKRPIYDTLFTDSLAKEGTEGDTYYSMMNWNLTKIHDGLMSK

SEQ ID NO: 6 - protein, 1873 aa, Enterococcus faecalis
MFGKNNKHMILKKGRKRVLKYSIKKLSVGVASVLVGVGLVFGATGIVNAQMGEGRLANYSASGNTFQENPGYTKNYNFSDLQFNPKAITGDVLQGNTIDFEVYGKHNIAASTANWEIRLQLDERLAQYVEKIQVDPKKGVGNSRRTFVRINDSLGRPTNIWKVNYIRANDGLFAGAETTDTQTAPNGVITFEKNLDEIFKEIGADNLKSDRLMYRIYLVSHQDDDKIVPGIESTGYFLTDQDDFYNKLDVSENNSDQFKHGSVNTKYEEANIQTKDGSGSTGANGAIILDHKLTKEKNFSYSTSAKGTPWYANYKIDERLVPYVSGIQMHMVQADKVAYNVAFESGKKVADLAIERREGHENYGMGSITDNDLTKLIDFANASPRPIVVRYVLQLTKPLDEILEEMKAADKIEENAPFGEDFIFDSWLSDTNKKLIQNTYGTGYYYLQDIDGDGNPDDKEESGDTNPYIGKPELEEVYDVDTTVKGKVFIHELAGTGHKAQLVDKEGTVLAEKTIAPNEKDGAPISDTVEFEFTGVDSSKLIAKDELKIQIVSPGFDKPEEGSTVIKESPKAVDKQTVVVGFKPDAKESIRNNKNLPEDAEYSWKTEPDTSNVTDSTKGIVTVKIGNRTFDVDVEFAVKASQAMENDATYVPITTTPETTIQSGKPTFDKPDVPLANDAFSVLDVYNKDFGNASVDANTGIVTFTPAKGVGESEPITGTIPIKIVYQDGSVGTTDLAVTVSKNIYENPGENIPAGYHKVTFTAGEGTSIESGTTVFAVKDGVSLPEDKLPVLKAKDGYTDAKWPEEATQPIKADDTEFVSSATKLDDIIENPGDNIPAGYHKVTFTAGEGTSIESGTTVFAVKDGVSLPEDKLPVLKAKDGYTDAKWPEEATQPIKADDTEFVSSATKLDDIIENPGDNIPAGYHKVTFTAGEGTSIESGTTVFAVKDGVSLPEDKLPVLKAKDGYTDAKWPGEATQPIKADDTEFVSSATKLDDKSDADKYTPEGQKVTTELNKEPDASEGIKNKKDLPKDAKYTWKEKVDISTAGNKKGTVVVTYSDGSSDEVEVDVTVTDNRSDADKYEPTVEGEKVEIGGKVDLTDNVTNLPTLPQGTTVTDVTPGGTIDTNTPGNYEGVIEVTYPDGTKDTVKVPVEVTDNRSDADKYEPTVEGEKVEIGGKVDLTDNVTNLPTLPQGTTVTDVTPGGTIDTNTPGNYEGVIEVTYPDGTKDTVKVPVEVTDNRSDADKYEPTVEGEKVEIGGKVDLTDNVTNLPTLPQGTTVTDVTPGGTIDTNTPGNYEGVIEVTYPDGTKDTVKVPVEVTDNRSDADKYTPMVEGEKVEIGGKVDLTDNVTNLPTLPQGTTVTDVTPGGTIDTNTPGNYEGVIEVTYPDGTKDTVKVPVEVTDNRSDADKYEPTVEGEKVEIGGKVDLTDNVTNLPTLPQGTTVTDVTPGGTIDTNTPGNYEGVIEVTYPDGTKDTVKVPVEVTDNRSDADKYEPTVEGEKVEIGGKVDLTDNVTNLPTLPQGTTITDVTPGGTIDTNTPGNYEGVIEVTYPDGTKDTVKVPVEVTDNRSDADKYEPTVEGEKVEIGGKVDLTDNVTNLPTLPQGTTITDVTPGGTIDTNTPGNYEGVIEVTYPDGTKDTVKVPVEVTDNRSDADKYTPKGQKVTTELNKEPEASDGIKNKSDLPKGTMYFWKEKVDVGIPGNKKATVVVIYPDGSKEEVEVVISVVDKKAPNKPQVDPITDGDKIVTGKTEPNADVTVTLPDGSQYHGTADKSGYFKVNVPKLEAGTKVKVTSTDESGNTSEPTDVVVSSNELNGGKGNGTDSKTNNNQDKKQFLKTYPKTGEVDSNIYTIAGGLILLGTLGLLGYEKWKKEDE

SEQ ID NO: 7 - protein, 91 aa, Enterococcus faecium
LYQGEMTSIKVSFSDKENQKIKPGDTITLTLPDELVGMTENDGSPRKINLNGLGEVFIYKDHVVATFNEKVESLHNVNGHFSFGIKTLITN

SEQ ID NO: 8 - protein, 509 aa, Enterococcus faecium
MKRLIGILATLVLTGMLAGCGASGEAGDSKEKLSVMTTFYPMYDFTKAIVGDEGEVELLIPAGTDSHDYEPSAKDMAKIQDTDIFVYNDENMETWVPAIQKTLQEGNVHTIKATEGMLLLPGSEEGHDHDHEHGEEGHTHEHELDPHVWLAPSLAIKQVASIRDQLIEAYPEKQEVWTKNAAAYTEKLQALHQLYQETFKQAKQRSFVTQHAAFNYLALEYGLNQVSIAGLSSSEEPSAARIAELKHFVKEHGINYIYFEENAKDSIARTLANEAGVSLEVLNPLEGLTNEQIENGENYLSIMEANLEALKKTTETENLLEDTLTPKKEKSVYNGYFEDEDVQDRSLSDWAGTWQSVDTYVEDGTFDPVFEYKEKLNQDKTAQEYKEYYTKGYQTDIASIKIGEKEMAFTFKDGATKRSNYRYVGKEILTYEAGNRGVRYLFEAEDKESGAFRYIQFSDHTIAPADSEHFHLYAGNESQEALLKEMENWPTFYPDTMDGREIAQAMLLH

SEQ ID NO: 9 - protein, 312 aa, Enterococcus faecium
MKKYLLTATVMIGALLFAACGNTNKEADKKEDLTIVTTFYPMYDFTKEIVGDEGNVKLLIPAGTEPHDFEPSAKERAEISDADVFVYNSSDMEFFVDSLKDSVDSKQTLMIEAAKGIDRLESQEADEHEESEEGHGHSHEYDPHVWLDPVLAIKEVRTIAEELGEKYPDKKETFTKNADAYIKKLEALDQKYSDELKDATNRTFVTQHAAFAYLANQYDLKQVAISGVSPDQEPTPSRLAELKEFVKKNNIKVIYFEENASSKVAETLSNETGVKLEVLNPLESLTNEQIKAGENYISVMEKNLKALKESIN

SEQ ID NO: 10 - protein, 1129 aa, Enterococcus faecium
MTTTGKKLKVIFMLIILSLSNFVPLSAIADTTDDPTVLETISAEVISDQSGKKALNIKLNANNTSAEKIEKEIGLVENYLSDVERKEGDGYAYQVNSGKITLEISSNTKQTIDLSFPIDPALYHSQANKLIVDNKEYDIIDETENKKDTDVSVPKPDEIEEESSKENENSVSPFTLPTLSLPAVSVPSNQTIPTEYTTDDQGTYPKASWQPTGNTNVLDHQGNKNGTNQWDGINSWNGDPNDRTHSYIEYGGTGNQADYAIRKYAKETSTPGLFDVYLNARGNVQKDITPLDLVLVVDWSGSMNDNNRIGEVKIGVDRFVDTLADSGITDKINMGYVGYSSEGYSYSNGAVQMGSFDSVKNQVKSITPSRTNGGTFTQKALRDAGSMLSVPNGHKKVIVLLTDGVPTFSYKVQRVHAQSSSNYYGTQFSNTQDRPGNTSLISRIYDAPDQNNLSRRIDSTFIATIGEAMALKERGIEIHGLGIQLQSDPAAGLSKAEVESRMRQMVSSDEKGDLYYESADHATDISEYLAKKAVQISATVSNGQINDPIAEPFIYQPGTLSVKSVGTSPTTVTPSISIEGNTIKSNQIYLGKDQEIQIHYQVRIQTENEDFHPNFWYQMNGRTTFQPNIDTNELAEFGIPSAKAPGVSLHIKKLWEEFDNNLADRPDQVTFEIQREHTTNAAAWKNGYIRIIKPAKDTTNTWERADIDKLSANSGESYQEILSLPQYNNQGQAFSYQTIKELPVPGYDSQQIDAMTWKNTKQFTPLNLKITKNSSTGEKDLIGAVFKLTGDSIDTLLTDHGDGTYSLPENVKLQKEMTYTLTETKAPEGHGLSKKTTWEIKIASDGTVTIDGKTVTTSDDTIQLTIENPFVEVPVAVRKYAMQGTDKEINLKGAAFSLQKKEANGTYQPIDSQTTNEKGLASFDSLTPGKYRVVETAGPAGYDTSPGNYEFQIDKYGKIIYTGKNTEMTNNVWTLTHQNRLKAFDLTVHKKEDNGQTLKGAKFRLQGPEMDLESPKDGQETDTFLFENLKPGTYTLTETFTPEGYQGLKEPVTIVIHEDGSIQVDGQDHESVLSPGAKNNQISLDITNQAKVPLPETGGIGRLGIYLVGMIGCAFSIWYLFLKKERGGS

SEQ ID NO: 11 - protein, 292 aa, Enterococcus faecium
MKTKKSLFLILAISLEVSKEKLSVVATNSILADMAKEVGTDKIDIHSIVPVGTDPHEYEVLPEDIKKASDADVILYNGLNLETGNSWFDNLMETAKKEEGKDYFAVSKNVEPLYLTSGEEHTKADPHAWLDLSNGIKYVQEIARIFSEKDAENATHYKRNAETYVEKLKELDTQAKESFASIEENKKLLVTSEGAFKYFSRAYDLPAAYIWEINTESQGTPDQMKAIIDQVRAKEVPVLFVERVAKETGLEIYDKLFTDSIAKEGEQGDSYYQMMKWNIETIHEGLSQTKES

SEQ ID NO: 12 - protein, 1900 aa, Enterococcus faecium
MVSKNNKRVFLEKTKKRVLKYSIKKLSVGVASVLVGVGLVLGTTELVQAQDEISPSTPLETAISSVQVGDKVASGNTFQENPGYTKNYNFSDLQFSPQELTGDTLKGNTIGFEVYGKHNIAASTKNWEIRLQLDERLAKYVEKIQVDPKKGIGSSRRTFVRINDSLGRPTNIWKVNYIRASDGLFAGAETTDTQTAPNGVITFEKSLDEIFKEIGIDNLKTDRLMYRIYLVSHQDDDKIVPGIDSTGYFLTDSDDFYNSLDVSENNPDQFKHGSVSAKYEEPNTQTKDGSGSTGANGAIILDHKLTKNYNFSYSASAKGTPWYANYKIDERLVPYVAGIQMHMVQADKVTYDVSFESGKKVADLAIERRKDHENYGVGSITDNDLTKLIDFANASPRPVVIRYVLQLTKPLDEILEDMKATAQVEENKPFGEDFIFDSWLSDTNKKLIQNTYGTGYYYLQDIDGDGNPDDKEESGDTNPYIGKPELEEVYDVDTTVKGKVFIHELAGTGHKAQLVDKEGTVLAEKTIAPNEKDGAPISDTVEFEFTGVDSSKLIAKDELKIQIVSPGFDKPEEGSTVIKESPKAVDKQTVVVGFKPDAKESIRNNKNLPEDAEYSWKTEPDTSNVTDSTKGIVTVKIGNRTFDVDVEFAVKASQAMENDATYVPITTTPETTVQSGKPTFDKPDVPLAKDAFSILDVYNKDFGNASVDANTGIVTFTPAKGVGESEPITGTIPIKIVYQDGSVGTTDLAVTVSKDIYENPGENIPAGYHKVTFTAGEGTSIESGTTVFAVKDGVSLPEDKLPVLKAKDGYTDAKWPEEATQPITADDTEFVSSATKLDDIIENPGENIPAGYHKVTFTAGEGTSIESGTTVFAVKDGVSLPEDKLPVLKAKDGYTDAKWPEEATQPITADDTEFVSSATKLDDIIENPGENIPAGYHKVTFTAGEGTSIESGTTVFAVKDGVSLPEDKLPVLKAKDGYTDAKWPEEATQPITADDTEFVSSATKLDDIIENPGENIPAGYHKVIFTAGEGTSIESGTTVFAVKDGVSLPEDKLPVLKAKDGYTDAKWPEEATQPITADDTEFVSSATKLDDIIENPGENIPAGYHKVIFTAGEGTSIESGTTVFAVKDGVSLPEDKLPVLKAKDGYTDAKWPEEATQPITADDTEFVSSATKLDDKSDADKYNPEGQKVTTELNKEPDASEGIKNKEDLPKDTKYTWKEKVDVSAAGNKKGTVVVTYSDGSSDEVEVDVTVTDNRSDADKYEPTVEGEKVEVGGTVDLTDNVTNLPTLPEGTTVTDVTPDGTIDTNTPGNYEGVIEVTYPDGTKDTVKVPVEVTDNRSDADKYTPMVEGEKVEVGGTVDLTDNVTNLPTLPEGTTVTDVTPDGTIDTNTPGNYEGVIEVTYPDGTKDTVKVPVEVTDNRSDADKYTPMVEGEKVEVGGTVDLTDNVTNLPTLPEGTTVTDVTPGGTIDTNTPGNYEGVIEVTYPDGTKDTVKVPVEVTDNRSDADKYEPTVEGEKVEVGGTVDLTDNVTNLPTLPEGTTVTDVTPGGTIDTNTPGNYEGVIEVTYPDGTKDTVKVPVEVTDNRSDADKYTPMVEGEKVEVGGTVDLTDNVTNLPTLPEGTTVTDVTPDGTIDTNTPGNYEGVIEVTYPDGTKDTVKVPVEVTDNRSDADKYNPEGQKVTTDLNKEPDASEGIKNKEDLPKGTTYTWKEKVDVSTAGNKKGIVVVTYPDGSKEEVEVTISVEDKKAPNKPQVDPITEGDQIVTGKTEPNAEVTVTLPNGSQYHGTADKNGNFTVKVPKLEAGTKVIVTATDESGNTSEPTNVVVSSNEKDSEKAVSKDNKTDNQGSKQNTNRGKSSPQKQSSKAYPKTGEIDSNIFTISGGLILLGTLGLLGYKNRKKENE

SEQ ID NO: 13 - protein, 337 aa, Enterococcus faecalis
GAELSKSSIVDKVELDHTTLYQGEMTSIKVSFSDKENQKIKPGDTITLTLPDALVGMTENDGSPRKINLNGLGEVFIYKDHVVATFNEKVESLHNVNGHFSFGIKTLITNSSQPNVIETDFGTATATQRLTIEGVTNTETGQIERDYPFFYKVGDLAGESNQVRWFLNVNLNKSDVTEDISIADRQGSGQQLNKESFTFDIVNDKETKYISLAEFEQQGYGKIDFVTDNDFNLRFYRNKARFTSFIVRYTSTITEAGQHQATFENSYDINYQLNNQDATNEKNTSQVKNVFVEGEASGNQNVEMPTEESLDIPLETIDEWEPKTPTSEQATETSEKT

SEQ ID NO: 14 - protein, 293 aa, Enterococcus faecalis
GQTTQSPKEKKELTVMTTFYPMYDFAKQVVGDEGEVELLIPAGTEPHDYEPSAKDLAKITDADVFVYNSKELETWVPNVIENLDTKKVSIVEASQSIQLMQGTEEEESGEEGHEGHNHSHELDPHVWLDPVLAQKEVTAIRDALIKKYPEKKAVFEKNTAAYLEKLTALDKEYQAAFAGAKNRTFVTQHAAFGYLAKQYGLTQEPIAGISPDQEPSPSRLAELKKYIKTNNVSVIYFEASASTKVAKTLADETGVELAVLNPLESLTQKEQEAGENYVSVMKENLAALQKSIH

SEQ ID NO: 15 - protein, 487 aa, Enterococcus faecalis
GSDKTADGKEKLSIVTTFYPMYDFTKNIVGDEGDVKLLIPAGSEPHDYEPSAKDMATIHDADVFVYHNENMESWVPKAAKGWKKGAPNVIKGTENMVLLPGSDEDGHDHDHEHGEEGHHHELDPHTWVSPHRAIQEVTNIKEQLVKLYPKKAKTFETNAEKYLTKLTALDKEFQTALKDAKQKSFVTQHAAFGYLALDYGLKQVPIAGLTPEQEPTAGRLAELKKYVTDNQIRYIYFEKNANDKIAKTLADEANVQLEVLNPLESLTQKQMDNGEDYLSVMKENLTALKKTTDTAGKEVQPETSEKTEKTVANGYFKDSEVAERTLADYAGNWQSVYPLLKDGTLDQVFDYKAKLKKDKTPAEYKTYYDAGYQTDVDHINITDSTIEFLVDGKPQKFTYKAAGYKILNYAKGNRGVRFLFETDDANAGRFKYVQFSDHNIAPTKAAHFHIFFGGDSQESLFNEMDNWPTYYPNDLSKQEIAQEMIAH

SEQ ID NO: 16 - protein, 840 aa, Enterococcus faecalis
ATEEVTNPFAEARMAPATLRANLALPLIAPQYTTDNSGTYPTANWQPTGNQNVLNHQGNKDGSAQWDGQTSWNGDPTNRTNSYIEYGGTGDQADYAIRKYARETTTPGLFDVYLNVRGNVQKEITPLDLVLVVDWSGSMNENNRIGEVQKGVNRFVDTLADSGITNNINMGYVGYSSDGYNNNAIQMGPFDTVKNPIKNITPSSTRGGTFTQKALRDAGDMLATPNGHKKVIVLLTDGVPTFSYKVSRVQTEADGRFYGTQFTNRQDQPGSTSYISGSYNAPDQNNINKRINSTFIATIGEAMALKQRGIEIHGLGIQLQSDPRANLSKQQVEDKMREMVSADENGDLYYESADYAPDISDYLAKKAVQISGTVVNGKVVDPIAEPFKYEPNTLSMKSVGPVQVQTLPEVSLTGATINSNEIYLGKGQEIQIHYQVRIQTESENFKPDFWYQMNGRTTFQPLATAPEKVDFGVPSGKAPGVKLNVKKIWEEYDQDPTSRPDNVIYEISRKQVTDTANWQTGYIKLSKPENDTSNSWERKNVTQLSKTADESYQEVLGLPQYNNQGQAFNYQTTRELAVPGYSQEKIDDTTWKNTKQFKPLDLKVIKNSSSGEKNLVGAVFELSGKNVQTTLVDNKDGSYSLPKDVRLQKGERYTLTEVKAPAGHELGKKTTWQIEVSEQGKVSIDGQEVTTTNQVIPLEIENKFSSLPIRIRKYTMQNGKQVNLAEATFALQRKNAQGSYQTVATQKTDTTGLSYFKISEPGEYRMVEQSGPLGYDTLAGNYEFTVDKYGKIHYAGKNIEENAPEWTLTHQNNLKPFDLTVHKKADNQTPLKGAKFRLTG

SEQ ID NO: 17 - protein, 285 aa, Enterococcus faecalis
AAEKKEKLAIVTTNSILSDLVKNVGQDKIELHSIVPIGTDPHEYEPLPEDIAKASEADILFFNGLNLETGGNGWFNKLMKTAKKVENKDYFSTSKNVTPQYLTSAGQEQTEDPHAWLDIENGIKYVENIRDVLVEKDPKNKDFYTENAKNYTEKLSKLHEEAKAKFADIPDDKKLLVTSEGAFKYFSKAYDLNAAYIWEINTESQGTPEQMTTIIDTIKKSKAPVLFVETSVDKRSMERVSKEVKRPIYDTLFTDSLAKEGTEGDTYYSMMNWNLTKIHDGLMSK

SEQ ID NO: 18 - protein, 370 aa, Enterococcus faecalis
GATGIVNAQMGEGRLANYSASGNTFQENPGYTKNYNFSDLQFNPKAITGDVLQGNTIDFEVYGKHNIAASTANWEIRLQLDERLAQYVEKIQVDPKKGVGNSRRTFVRINDSLGRPTNIWKVNYIRANDGLFAGAETTDTQTAPNGVITFEKNLDEIFKEIGADNLKSDRLMYRIYLVSHQDDDKIVPGIESTGYFLTDQDDFYNKLDVSENNSDQFKHGSVNTKYEEANIQTKDGSGSTGANGAIILDHKLTKEKNFSYSTSAKGTPWYANYKIDERLVPYVSGIQMHMVQADKVAYNVAFESGKKVADLAIERREGHENYGMGSITDNDLTKLIDFANASPRPIVVRYVLQLTKPLDEILEEMKAADK

(19) United States
(12) Patent Application Publication
(10) Pub. No.: US 2025/0269004 A1
(43) Pub. Date: Aug. 28, 2025

(54) ENTEROCOCCUS FAECALIS VACCINE AND USES THEREOF

(71) Applicants: Vaxcyte, Inc., San Carlos, CA (US); University of Florida Research Foundation, Inc., Gainesville, FL (US)

(72) Inventors: Jeffery C. Fairman, Mountain View, CA (US); Aym M. Berges, San Carlos, CA (US); Angie A. Sedra, San Carlos, CA (US); Jose A. Lemos, Gainesville, FL (US)

(21) Appl. No.: 18/857,949

(22) PCT Filed: Apr. 18, 2023
(86) PCT No.: PCT/US2023/065878

§ 371 Date: Oct. 18, 2024

Related U.S. Application Data
(60) Provisional application No. 63/332,110, filed on Apr. 18, 2022.

(51) Int. Cl. A61K 39/09; A61K 9/00; A61K 39/00; A61P 31/04

(52) U.S. Cl. CPC A61K 39/09; A61K 9/0019; A61P 31/04; A61K 2039/55505; A61K 2039/55572; A61K 2039/55577; A61K 2039/575

(57) ABSTRACT

The present disclosure provides immunogenic compositions comprising at least one recombinant polypeptide antigen derived from an Enterococcus bacterium (e.g., E. faecalis, E. faecium, E. durans). The disclosure further provides methods, and uses of the immunogenic compositions, for protecting or treating a subject from infection by an Enterococcus bacterium. Such infections may cause, or worsen, conditions such as root canal failure, endocarditis, bacteremia, urinary tract infections, prostatitis, intraabdominal infection, cellulitis, dysbiotic gastrointestinal tract, prosthetic joint infection, or wound infections.

### CROSS-REFERENCE TO RELATED APPLICATIONS

This application claims the benefit of U.S. Provisional Application No. 63/332,110, filed Apr. 18, 2022, the disclosure of which is hereby incorporated by reference in its entirety.

### DESCRIPTION OF THE TEXT FILE SUBMITTED ELECTRONICALLY

The contents of the electronic sequence listing (STRO_023_01WO_SeqList_ST26.xml; Size: 28,215 bytes; and Date of Creation: Mar. 15, 2023) are herein incorporated by reference in its entirety.

### TECHNICAL FIELD

This invention relates generally to the field of microbiology and vaccine development.

### BACKGROUND

There are approximately 15 million root canal procedures performed in the US every year, and ˜20% (3 million) fail due to infection. This infection is primarily caused by Enterococcus faecalis, a species of bacteria from the large Enterococcus genus of gram-positive bacteria. The cost of follow-up treatment or implant for a failed root canal currently averages $2,000. Assuming a 50% reimbursement, the cost to insurance companies would be around $3 billion annually. Additionally, E. faecalis causes other significant, and sometimes fatal, opportunistic human diseases, including endocarditis, urinary tract infections, prostatitis, intra-abdominal infection, cellulitis, and wound infections.

Species of the Enterococcus genus also have a high intrinsic antibiotic resistance, and some strains have gained resistance to powerful second-line antibiotics such as vancomycin (see e.g., Arias, C. A., et al. Nat. Rev. Microbiol. 2012, 266-278; Puchter, L., et al. Antimicrob. Resist. Infect. Control 2018). A vaccine that reduces the rate of root canal failure, or any of the other conditions described herein, would have favorable pharmacoeconomic and human health implications. However, there is presently no approved, or promising, candidate vaccine. Accordingly, there is a need for a first-in-class preventative medicines and treatments for Enterococcus infection.

### SUMMARY

Generally, an immunogenic composition for an Enterococcus infection comprises at least one recombinant Enterococcus polypeptide antigen or fragment thereof. The immunogenic composition may comprise at least one recombinant Enterococcus faecalis polypeptide antigen or fragment thereof. The polypeptide antigens may, for example be an Ace polypeptide antigen, an AdcA polypeptide antigen, an AdcAII polypeptide antigen, an EbpA polypeptide antigen, an EfaA polypeptide antigen, an Esp polypeptide antigen, or any suitable recombinant polypeptide antigen derived from an Enterococcus protein.

An immunogenic composition may, for example, comprise a first and a second recombinant Enterococcus polypeptide antigen. An immunogenic composition may also comprise a first, second, and third recombinant Enterococcus polypeptide antigen. Some immunogenic compositions may comprise a first, second, third, and fourth recombinant Enterococcus polypeptide antigen. Some immunogenic compositions may comprise a first, second, third, fourth, and fifth recombinant Enterococcus polypeptide antigen. An immunogenic composition may also comprise 6 or greater recombinant Enterococcus polypeptide antigens.

Generally, a method for inducing a protective immune response against an Enterococcus bacterium may include administering an immunogenic composition comprising at least one recombinant Enterococcus polypeptide antigen. Generally, a use of an immunogenic composition may induce a protective immune response against an Enterococcus bacterium. Generally, an immunogenic composition, or method or use thereof, might be useful for a patient suffering from, or at risk for suffering from, root canal failure, endocarditis, bacteremia, urinary tract infections, prostatitis, intraabdominal infection, cellulitis, dysbiotic gastrointestinal tract, prosthetic joint infection, or wound infections.

### BRIEF DESCRIPTION OF THE DRAWINGS

FIG. 1 shows the total average protein and average soluble protein of the expressed polypeptide antigens (μg/mL) as well as the accompanying SDS page gels.

FIG. 2 shows the relative expression of two different EbpA constructs: full-length and the EbpA-IpaH domain.

FIG. 3 shows gels after expression/purification and TEV cleavage for an E. faecalis EfaA polypeptide antigen.

FIG. 4 shows gels after expression/purification and TEV cleavage for an E. faecalis Ace polypeptide antigen.

FIG. 5 shows gels after expression/purification and TEV cleavage for an E. faecalis Esp polypeptide antigen.

FIG. 6 shows gels after expression/purification and TEV cleavage for an E. faecalis AdcA polypeptide antigen.

FIG. 7 shows gels after expression/purification and TEV cleavage for an E. faecalis AdcAII polypeptide antigen.

FIG. 8 shows gels after expression/purification and TEV cleavage for an E. faecalis EpbA polypeptide antigen.

FIG. 9 shows the chromatographs for the SEC-MALS molecular weight analysis for polypeptide antigens of SEQ ID NOs: 12-18.

FIGS. 10A and 10B show SDS-PAGE and SEC-MALS data, respectively, for a batch of expressed polypeptide antigens used in animal studies.

FIG. 11A shows that AdcA is relatively stable to freeze-thaw cycles, with only a small decrease in the main peak magnitude and molecular weight. FIG. 11B shows that AdcAII is also relatively stable to freeze-thaw cycles, with only a small decrease in the main peak magnitude and a slight increase in molecular weight. FIG. 11C shows that Ace is stable to freeze-thaw cycles. FIG. 11D shows the stability of two different Esp samples to freeze-thaw cycles. FIG. 11D shows that Esp is stable when 10% glycerol is added to the storage buffer. FIGS. 11E and 11F show that EfaA and EpbA, respectively, are relatively stable to freeze-thaw cycles.

FIG. 12A shows the resultant immunoblot generated from probing cell lysates generated from: wild-type E. faecalis OG1RF, E. faecalis OG1RF AAdcACB/AAdcA double null mutant, as well as 0.25 μg each of the AdcA and AdcAII antigens used to generate the mouse antisera, with the AdcA mouse antisera. FIG. 12B shows the resultant immunoblot generated from probing cell lysates generated from: wild-type E. faecalis OG1RF, E. faecalis OG1RF AAdcACB/AAdcA double null mutant, as well as 0.25 μg each of the AdcA and AdcAII antigens used to generate the mouse antisera, with the AdcAII mouse antisera.

FIG. 13 shows the survival data from a G. mellonella immunization model utilizing rabbit anti-sera derived from exemplary E. faecalis polypeptide antigen treatment arms.

FIG. 14A shows the dosing schedule for an immunization study in rabbits utilizing exemplary E. faecalis immunogenic compositions. FIG. 14B shows each of the immunogenic compositions tested in the rabbit cohorts.

FIG. 15 shows that anti-sera derived from animals dosed with individual E. faecalis polypeptide antigens raises antigen-specific IgG titers relative to pre-immunized animals and relative to an alum control.

FIG. 16 provides an overview of an opsonophagocytic (OPA) assay used to assess antibody-dependent killing efficacy by neutrophils post-immunization with select E. faecalis polypeptide antigens.

FIG. 17 shows opsonizing/neutralizing effect of anti-sera antibodies on neutrophil-mediated killing of E. faecalis in vitro.

FIG. 18 shows a graphical overview of a passive immunization mouse peritonitis model.

FIG. 19 shows a summary of colony forming unit (CFU) data generated from a mouse peritoneal (peritonitis) infection model for two body locations (peritoneal cavity and spleen) examined for the presence of active E. faecalis after passive immunization.

FIG. 20 shows a summary of colony forming unit data generated from a mouse peritoneal (peritonitis) infection model for kidneys examined for the presence of active E. faecalis after passive immunization.

FIG. 21A, FIG. 21B, and FIG. 21C show terminal bleed IgG titers for ten treatment groups in a passive immunization study to determine the protection efficiency of anti-sera generated against E. faecalis antigen combinations

FIG. 22 shows graphical overview of a passive immunization mouse peritonitis model without a second booster shot of sera.

FIG. 23A, FIG. 23B, FIG. 23C, FIG. 23D, FIG. 23E, FIG. 23F, FIG. 23G, FIG. 23H, show the survival data generated from a G. mellonella immunization model utilizing rabbit anti-sera derived from exemplary E. faecalis polypeptide antigen treatment arms.

FIG. 24A and FIG. 24B show the effectiveness of E. faecalis OG1RF antigen-specific anti-sera against clinically relevant E. faecalis strains and E. faecium strains, respectively. FIG. 24C shows a summary of colony forming unit data generated from a mouse peritoneal (peritonitis) infection model for the presence of active E. faecalis and E. faecium after passive immunization.

### DETAILED DESCRIPTION

Although vaccines against enterococcal infections have been explored previously (see e.g., Kalfopoulou, E., et al. Cells, 2020, 2397), as discussed above, neither a preventative nor therapeutic vaccine has been successfully developed, despite the potentially significant human health repercussions posed by pathogenic Enterococcus species (e.g., E. faecalis, E. faecium, E. durans).

### Immunogenic Compositions

In some embodiments, the present disclosure provides an immunogenic composition comprising at least one recombinant Enterococcus (e.g., E. faecalis, E. faecium, E. durans) polypeptide antigen or a fragment thereof.

As used herein, the term “immunogenic” refers to the ability of an antigen (e.g., a polypeptide), to elicit an immune response, including either a humoral or a cellular immune response, and preferably both. In some embodiments, the subject will display either a therapeutic or protective immunological response to administration of an “effective amount” or “immunologically effective amount” of an immunogenic composition herein such that resistance to new infection will be enhanced and/or the clinical severity of the disease will be reduced. The immunological response will normally be demonstrated by alleviation or elimination of at least one symptom associated with the infection.

The “immunologically effective amount” or “effective amount” of the immunogenic composition is an amount that, either as a single dose or as part of a series of two or more doses, is effective for treating or preventing Enterococcus infection or an Enterococcus-related disease or condition. The amount administered will vary according to several factors, including the overall health and physical condition of the subject, the subject's age, the capacity of the subject's immune system to synthesize relevant antibodies, the form of the composition (e.g., injectable liquid, nasal spray, etc.), and other factors known to the medical practitioner overseeing administration.

The term “treating” refers to therapeutic treatment by the administration of an immunogenic composition where the objective is to lessen or eliminate infection. For example, “treating” may include directly affecting, suppressing, inhibiting, and eliminating infection, as well as reducing the severity of, delaying the onset of, and/or reducing symptoms associated with an infection. Unless otherwise indicated explicitly or implied by context, the term “treating” encompasses “preventing” (or prophylaxis or prophylactic treatment) where “preventing” may refer to reducing the risk that a subject will develop an infection, delaying the onset of symptoms, preventing relapse of an infection, or preventing the development of infection.

As used herein, “recombinant” polypeptides refer to polypeptides produced by recombinant DNA techniques (e.g., produced from cells transformed by an exogenous DNA construct encoding the desired polypeptide). In some embodiments, one or more recombinant polypeptides are synthesized using scalable cell-free protein synthesis (CFPS), as described in U.S. Pat. Nos. 9,040,253, 9,650,621, and Murray, et al. 2013 Current Opin. Chem. Biol. 17 (3): 420-26, all of which are incorporated by reference herein. An expressed polypeptide may contain a leader sequence to assist in isolation and/or purification. For instance, the peptide may have a terminal His (e.g., 6xHis) tag. The peptide may have a cleavable linker (e.g., protease cleavable) between a terminal tag and the primary polypeptide sequence. It should be understood that the polypeptides of the immunogenic compositions described herein are recombinant polypeptides.

In some embodiments, the present disclosure provides a purified recombinant Enterococcus polypeptide antigen. As used herein, when the term “purified” is used in reference to a molecule, it means that the concentration of the molecule being purified has been increased relative to the concentration of the molecule in the environment in which it is produced. The term may also refer to purification of a chemically synthesized molecule from a reaction mixture in which the molecule has been generated as a reaction product. As used herein, when the term “isolated” is used in reference to a molecule, the term means that the molecule has been removed from its native environment. For example, a polynucleotide or a polypeptide naturally present in a living organism is not “isolated,” but the same polynucleotide or polypeptide separated from the coexisting materials in its natural state is “isolated.” An isolated moiety, whether separated from a native environment or from a non-natural environment (e.g., recombinant expression, cell-free expression, chemical synthesis, etc.), is preferably at least about 1% pure, 5% pure, 10% pure, 20% pure, 30% pure, 40% pure, 50% pure, 60% pure, 70% pure, 80% pure, 90% pure, 95% pure, or 99% pure, or 100% pure. As used herein, the term “% pure” indicates the percentage of a composition that is made up of the molecule of interest, by weight.

As used herein, the term “polypeptide” is intended to include any structure comprised of one or more amino acids, and thus includes dipeptides, oligopeptides, polypeptides, and/or polypeptide fragments thereof. The amino acids forming all or a part of a polypeptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine(S), threonine (T), valine (V), tryptophan (W), and tyrosine (Y), as well as non-conventional amino acids such as isomers and modifications of the conventional amino acids, (e.g., D-amino acids, non-protein amino acids, post-translationally modified amino acids, enzymatically modified amino acids, β-amino acids), constructs or structures designed to mimic amino acids (e.g., α,α-disubstituted amino acids, N-alkyl amino acids, lactic acid, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine), and other non-conventional amino acids.

In some embodiments, the polypeptide antigen described herein is an Enterococcus membrane-associate polypeptide. For example, in some embodiments, the polypeptide antigen is an adhesin to collagen of Enterococci (e.g., an “Ace” polypeptide antigen from E. faecalis, E. faceium, E. durans). In certain embodiments, the polypeptide antigen is a zinc ABC transporter substrate-binding lipoprotein (e.g., an “AdcA” polypeptide antigen from E. faecalis, E. faceium, E. durans, an “AdcAII” polypeptide antigen from E. faecalis, E. faceium, E. durans). In certain embodiments, the polypeptide antigen is an endocarditis and biofilm-associate pili protein (e.g., an “EbpA” polypeptide antigen from E. faecalis, E. faceium, E. durans). In certain embodiments, the polypeptide antigen is an ABC superfamily ATP binding cassette transporter (e.g., an “EfaA” polypeptide antigen from E. faecalis, E. faceium, E. durans). In some embodiments, the polypeptide antigen is an enterococcal surface protein (e.g., an “Esp” polypeptide antigen from E. faecalis, E. faceium, E. durans).

Amino acid sequences of exemplary Enterococcus polypeptides are provided below in Table 1.

TABLE 1
Exemplary Enterococcus Polypeptide Antigens

| Seq | Name/ | |
|---|---|---|
| ID | description | Amino acid sequence |
| 1 | Ace WT - | MTKSVKFLVLLLVMILPIAGALLIGPISFGAELSKSSIVDKVELDHTTLYQGEMTSI |
| | E. faecalis | KVSFSDKENQKIKPGDTITLTLPDALVGMTENDGSPRKINLNGLGEVFIYKDHVVAT |
| | | FNEKVESLHNVNGHESFGIKTLITNSSQPNVIETDEGTATATQRLTIEGVINTETGQ |
| | | IERDYPFFYKVGDLAGESNQVRWFLNVNLNKSDVTEDISIADRQGSGQQLNKESFTF |
| | | DIVNDKETKYISLAEFEQQGYGKIDFVTDNDENLRFYRNKARFTSFIVRYTSTITEA |
| | | GQHQATFENSYDINYQLNNQDATNEKNTSQVKNVFVEGEASGNQNVEMPTEESLDIP |
| | | LETIDEWEPKTPTSEQATETSEKTGATETAESSQPEVHVSPTEEENPDEGGTLGTIE |
| | | PIIPEKPSVTTEENGVTETAEGSQPEVHVSPTEEENPDESETLGTITPIIPEKPSVT |
| | | TEENGATETAESSQPEVHVSPAEEENPDESETLGTITPIIPEKPSVTTEENGTTETA |
| | | ESSQPEVHVSPTEEENLDESETLGTIEPIIPEKPSVTTEENGTTETAESSQPEVHVS |
| | | PTKEITTTEKKQPSTETTVEKNKNVTSKNQPQILNAPLNTVKNEGSPQLAPQLLSEP |
| | | IQKLNEANGQRELPKTGTTKTPFMLIAGILASTFAVLGVSYLQIRKN |
| 2 | AdcA WT - | MTKIYRRLIIGVTLAISAFLLASCGQTTQSPKEKKELTVMTTFYPMYDFAKQVVGDE |
| | E. faecalis | GEVELLIPAGTEPHDYEPSAKDLAKITDADVFVYNSKELETWVPNVIENLDTKKVSI |
| | | VEASQSIQLMQGTEEEESGEEGHEGHNHSHELDPHVWLDPVLAQKEVTAIRDALIKK |
| | | YPEKKAVEEKNTAAYLEKLTALDKEYQAAFAGAKNRTFVTQHAAFGYLAKQYGLTQE |
| | | PIAGISPDQEPSPSRLAELKKYIKTNNVSVIYFEASASTKVAKTLADETGVELAVLN |
| | | PLESLTQKEQEAGENYVSVMKENLAALQKSIH |
| 3 | AdcAII WT - | MKKFTLPLLAALSLILFGACGKTNTSDKTADGKEKLSIVTTFYPMYDFTKNIVGDEG |
| | E. faecalis | DVKLLIPAGSEPHDYEPSAKDMATIHDADVFVYHNENMESWVPKAAKGWKKGAPNVI |
| | | KGTENMVLLPGSDEDGHDHDHEHGEEGHHHELDPHTWVSPHRAIQEVTNIKEQLVKL |
| | | YPKKAKTFETNAEKYLTKLTALDKEFQTALKDAKQKSFVTQHAAFGYLALDYGLKQV |
| | | PIAGLTPEQEPTAGRLAELKKYVTDNQIRYIYFEKNANDKIAKTLADEANVQLEVLN |
| | | PLESLTQKQMDNGEDYLSVMKENLTALKKTTDTAGKEVQPETSEKTEKTVANGYFKD |
| | | SEVAERTLADYAGNWQSVYPLLKDGTLDQVFDYKAKLKKDKTPAEYKTYYDAGYQTD |
| | | VDHINITDSTIEFLVDGKPQKFTYKAAGYKILNYAKGNRGVRFLFETDDANAGREKY |
| | | VQFSDHNIAPTKAAHFHIFFGGDSQESLFNEMDNWPTYYPNDLSKQEIAQEMIAH |
| 4 | EbpA WT - | MLIHKRKERTDIKLLKKFGVFGVVELLVSSYFIPLIGYAETAKEVEITSAQMITDEN |
| | E. faecalis | DKTNINIELNLLNQTEQPLQREIQLKNAQFMDTAVIEKDGYSYQVINGTLYLTLDAQ |
| | | VKKPVQLSLAVEQSSLQTAQPPKLLYENNEYDVSVTSEKITVEDSAKESTEPEKITV |
| | | PENTKETNKNDSAPENTEQPTATEEVTNPFAEARMAPATLRANLALPLIAPQYTTDN |
| | | SGTYPTANWQPTGNQNVLNHQGNKDGGTQWDGQTSWNGDPTNRTNSYIEYGGTGDQA |
| | | DYAIRKYARETTTPGLFDVYLNVRGNVQKEITPLDLVLVVDWSGSMNENNRIGEVQK |
| | | GVNRFVDTLADSGITNNINMGYVGYSSDSYNNNAIQMGPFDTVKNPIKNITPSSTRG |
| | | GTFTQKALRDAGDMLATPNGHKKVIVLLTDGVPTFSYKVSRVQTEADGRFYGTQFTN |
| | | RQDQPGSTSYISGSYNAPDQNNINKRINSTFIATIGEAMALKQRGIEIHGLGIQLQS |
| | | DPRANLSKQQVEDKMREMVSADENGDLYYESADYAPDISDYLAKKAVQISGTVVNGK |
| | | VVDPIAEPFKYEPNTLSMKSVGPVQVQTLPEVSLTGATINSNEIYLGKGQEIQIHYQ |
| | | VRIQTESENEKPDFWYQMNGRTTFQPLATAHEKVDFGVPSGKAPGVKLNVKKIWEEY |
| | | DQDPTSRPDNVIYEISRKQVTDTANWQTGYIKLSKPENDTSNSWERKNVTQLSKTAD |
| | | ESYQEVLGLPQYNNQGQAFNYQTTRELAVPGYSQEKIDDTTWKNTKQFKPLDLKVIK |
| | | NSSSGEKNLVGAVFELSGKNVQTTLVDNKDGSYSLPKDVRLQKGERYTLTEVKAPAG |
| | | HELGKKTTWQIEVSEQGKVSIDGQEVTTTNQVIPLEIENKFSSLPIRIRKYTMQNGK |
| | | QVNLAEATFALQRKNAQGSYQTVATQKTDTAGLSYFKISEPGEYRMVEQAGPLGYDT |
| | | LAGNYEFTVDKYGKIHYAGKNIEENAPEWTLTHQNHLKPFDLTVHKKADNQTPLKGA |
| | | KFRLTGPDTDIELPKDGKETDTFVFENLKPGKYVLTETFTPEGYQGLKEPIELIIRE |
| | | DGSVTIDGEKVADVLISGEKNNQITLDVTNQAKVPLPETGGIGRLWFYLIAISTFVI |
| | | AGVYLFIRRPEGSV |
| 5 | EfaA WT - | MKKFSLFFLTLLAGLTLAACGNQAAEKKEKLAIVTTNSILSDLVKNVGQDKIELHSI |
| | E. faecalis | VPIGTDPHEYEPLPEDIAKASEADILFFNGLNLETGGNGWENKLMKTAKKVENKDYF |
| | | STSKNVTPQYLTSAGQEQTEDPHAWLDIENGIKYVENIRDVLVEKDPKNKDFYTENA |
| | | KNYTEKLSKLHEEAKAKFADIPDDKKLLVTSEGAFKYFSKAYDLNAAYIWEINTESQ |
| | | GTPEQMTTIIDTIKKSKAPVLFVETSVDKRSMERVSKEVKRPIYDTLFTDSLAKEGT |
| | | EGDTYYSMMNWNLTKIHDGLMSK |
| 6 | Esp WT - | MFGKNNKHMILKKGRKRVIKYSIKKESVGVASVEVGVGLVEGATGIVNAQMGEGRLA |
| | E. faecalis | NYSASGNTFQENPGYTKNYNESDLQFNPKAITGDVLQGNTIDFEVYGKHNIAASTAN |
| | | WEIRLQLDERLAQYVEKIQVDPKKGVGNSRRTFVRINDSLGRPTNIWKVNYIRANDG |
| | | LFAGAETTDTQTAPNGVITFEKNLDEIFKEIGADNIKSDRLMYRIYLVSHQDDDKIV |
| | | PGIESTGYFLTDQDDFYNKLDVSENNSDQFKHGSVNTKYEEANIQTKDGSGSTGANG |
| | | AIILDHKLIKEKNFSYSTSAKGTPWYANYKIDERLVPYVSGIQMHMVQADKVAYNVA |
| | | FESGKKVADLAIERREGHENYGMGSITDNDLTKLIDFANASPRPIVVRYVLQLTKPL |
| | | DEILEEMKAADKIEENAPFGEDFIFDSWLSDINKKLIQNTYGTGYYYLQDIDGDGNP |
| | | DDKEESGDINPYIGKPELEEVYDVDTTVKGKVEIHELAGTGHKAQLVDKEGTVLAEK |
| | | TIAPNEKDGAPISDTVEFEFTGVDSSKLIAKDELKIQIVSPGEDKPEEGSTVIKESP |
| | | KAVDKQTVVVGFKPDAKESIRNNKNIPEDAEYSWKTEPDTSNVTDSTKGIVTVKIGN |
| | | RTFDVDVEFAVKASQAMENDATYVPITTTPETTIQSGKPTEDKPDVPLANDAFSVLD |
| | | VYNKDFGNASVDANTGIVTFTPAKGVGESEPITGTIPIKIVYQDGSVGTTDLAVTVS |
| | | KNIYENPGENIPAGYHKVTFTAGEGTSIESGTTVEAVKDGVSLPEDKLPVLKAKDGY |
| | | TDAKWPEEATQPIKADDTEEVSSATKLDDIIENPGDNIPAGYHKVTFTAGEGTSIES |
| | | GTTVEAVKDGVSLPEDKLPVLKAKDGYTDAKWPEEATQPIKADDTEFVSSATKLDDI |
| | | IENPGDNIPAGYHKVTFTAGEGTSIESGTTVFAVKDGVSLPEDKLPVLKAKDGYTDA |
| | | KWPGEATQPIKADDTEFVSSATKLDDKSDADKYTPEGQKVTTELNKEPDASEGIKNK |
| | | KDLPKDAKYTWKEKVDISTAGNKKGTVVVTYSDGSSDEVEVDVTVTDNRSDADKYEP |
| | | TVEGEKVEIGGKVDLTDNVTNLPTLPQGTTVTDVTPGGTIDINTPGNYEGVIEVTYP |
| | | DGTKDTVKVPVEVTDNRSDADKYEPTVEGEKVEIGGKVDLTDNVTNLPTLPQGTTVT |
| | | DVTPGGTIDINTPGNYEGVIEVTYPDGTKDTVKVPVEVTDNRSDADKYEPTVEGEKV |
| | | EIGGKVDLTDNVTNLPTLPQGTTVTDVTPGGTIDTNTPGNYEGVIEVTYPDGTKDTV |
| | | KVPVEVTDNRSDADKYTPMVEGEKVEIGGKVDLTDNVTNLPTLPQGTTVTDVTPGGT |
| | | IDTNTPGNYEGVIEVTYPDGTKDTVKVPVEVTDNRSDADKYEPTVEGEKVEIGGKVD |
| | | LIDNVTNLPTLPQGTTVIDVTPGGTIDINTPGNYEGVIEVTYPDGTKDTVKVPVEVT |
| | | DNRSDADKYEPTVEGEKVEIGGKVDLTDNVTNLPTLPQGTTITDVTPGGTIDTNTPG |
| | | NYEGVIEVTYPDGTKDTVKVPVEVTDNRSDADKYEPTVEGEKVEIGGKVDLTDNVTN |
| | | LPTLPQGTTITDVTPGGTIDTNTPGNYEGVIEVTYPDGTKDTVKVPVEVTDNRSDAD |
| | | KYTPKGQKVTTELNKEPEASDGIKNKSDLPKGTMYFWKEKVDVGIPGNKKATVVVIY |
| | | PDGSKEEVEVVISVVDKKAPNKPQVDPITDGDKIVTGKTEPNADVTVTLPDGSQYHG |
| | | TADKSGYFKVNVPKLEAGTKVKVISTDESGNTSEPTDVVVSSNELNGGKGNGTDSKT |
| | | NNNQDKKQFLKTYPKTGEVDSNIYTIAGGLILLGTLGLLGYEKWKKEDE |
| 7 | Ace WT - | LYQGEMTSIKVSFSDKENQKIKPGDTITLTLPDELVGMTENDGSPRKINLNGLGEVF |
| | E. faecium | IYKDHVVATFNEKVESLHNVNGHFSFGIKTLITN |
| 8 | AdcA WT - | MKRLIGILATLVLTGMLAGCGASGEAGDSKEKLSVMTTFYPMYDETKAIVGDEGEVE |
| | E. faecium | LLIPAGTDSHDYEPSAKDMAKIQDTDIFVYNDENMETWVPAIQKTLQEGNVHTIKAT |
| | | EGMLLLPGSEEGHDHDHEHGEEGHTHEHELDPHVWLAPSLAIKQVASIRDQLIEAYP |
| | | EKQEVWTKNAAAYTEKLQALHQLYQETFKQAKQRSFVTQHAAFNYLALEYGLNQVSI |
| | | AGLSSSEEPSAARIAELKHFVKEHGINYIYFEENAKDSIARTLANEAGVSLEVINPL |
| | | EGLTNEQIENGENYLSIMEANLEALKKTTETENLLEDTLTPKKEKSVYNGYFEDEDV |
| | | QDRSLSDWAGTWQSVDTYVEDGTFDPVFEYKEKLNQDKTAQEYKEYYTKGYQTDIAS |
| | | IKIGEKEMAFTFKDGATKRSNYRYVGKEILTYEAGNRGVRYLFEAEDKESGAFRYIQ |
| | | FSDHTIAPADSEHFHLYAGNESQEALLKEMENWPTFYPDTMDGREIAQAMLLH |
| 9 | AdcAII WT - | MKKYLLTATVMIGALLFAACGNTNKEADKKEDLTIVTTFYPMYDFTKEIVGDEGNVK |
| | E. faecium | LLIPAGTEPHDFEPSAKERAEISDADVFVYNSSDMEFFVDSLKDSVDSKQTLMIEAA |
| | | KGIDRLESQEADEHEESEEGHGHSHEYDPHVWLDPVLAIKEVRTIAEELGEKYPDKK |
| | | ETFTKNADAYIKKLEALDQKYSDELKDATNRTFVTQHAAFAYLANQYDLKQVAISGV |
| | | SPDQEPTPSRLAELKEFVKKNNIKVIYFEENASSKVAETLSNETGVKLEVLNPLESL |
| | | TNEQIKAGENYISVMEKNLKALKESIN |
| 10 | EbpA WT - | MTTTGKKLKVIEMLIILSLSNFVPLSAIADTTDDPTVLETISAEVISDQSGKKALNI |
| | E. faecium | KLNANNTSAEKIEKEIGLVENYLSDVERKEGDGYAYQVNSGKITLEISSNTKQTIDL |
| | | SFPIDPALYHSQANKLIVDNKEYDIIDETENKKDTDVSVPKPDEIEEESSKENENSV |
| | | SPFTLPTLSLPAVSVPSNQTIPTEYTTDDQGTYPKASWQPTGNTNVLDHQGNKNGTN |
| | | QWDGINSWNGDPNDRTHSYIEYGGTGNQADYAIRKYAKETSTPGLEDVYLNARGNVQ |
| | | KDITPLDLVLVVDWSGSMNDNNRIGEVKIGVDRFVDTLADSGITDKINMGYVGYSSE |
| | | GYSYSNGAVQMGSFDSVKNQVKSITPSRTNGGTFTQKALRDAGSMLSVPNGHKKVIV |
| | | LLTDGVPTFSYKVQRVHAQSSSNYYGTQFSNTQDRPGNTSLISRIYDAPDQNNLSRR |
| | | IDSTFIATIGEAMALKERGIEIHGLGIQLQSDPAAGLSKAEVESRMRQMVSSDEKGD |
| | | LYYESADHATDISEYLAKKAVQISATVSNGQINDPIAEPFIYQPGTLSVKSVGTSPT |
| | | TVTPSISIEGNTIKSNQIYLGKDQEIQIHYQVRIQTENEDFHPNFWYQMNGRTTFQP |
| | | NIDTNELAEFGIPSAKAPGVSLHIKKLWEEFDNNLADRPDQVTFEIQREHTTNAAAW |
| | | KNGYIRIIKPAKDTTNTWERADIDKLSANSGESYQEILSLPQYNNQGQAFSYQTIKE |
| | | LPVPGYDSQQIDAMTWKNTKQFTPLNLKITKNSSTGEKDLIGAVFKLTGDSIDTLLT |
| | | DHGDGTYSLPENVKLQKEMTYTLTETKAPEGHGLSKKTTWEIKIASDGTVTIDGKTV |
| | | TTSDDTIQLTIENPFVEVPVAVRKYAMQGTDKEINLKGAAFSLQKKEANGTYQPIDS |
| | | QTTNEKGLASFDSLTPGKYRVVETAGPAGYDTSPGNYEFQIDKYGKIIYTGKNTEMT |
| | | NNVWTLTHQNRLKAFDLTVHKKEDNGQTLKGAKERLQGPEMDLESPKDGQETDTELE |
| | | ENLKPGTYTLTETFTPEGYQGLKEPVTIVIHEDGSIQVDGQDHESVLSPGAKNNQIS |
| | | LDITNQAKVPLPETGGIGRLGIYLVGMIGCAFSIWYLELKKERGGS |
| 11 | EfaA WT - | MKTKKSLFLILAISLEVSKEKLSVVAINSILADMAKEVGIDKIDIKSIVPVGTDPH |
| | E. faecium | EYEVLPEDIKKASDADVILYNGLNLETGNSWEDNLMETAKKEEGKDYFAVSKNVEP |
| | | LYLTSGEEHTKADPHAWLDLSNGIKYVQEIARIFSEKDAENATHYKRNAETYVEKL |
| | | KELDTQAKESFASIEENKKLLVTSEGAFKYESRAYDLPAAYIWEINTESQGTPDQM |
| | | KAIIDQVRAKEVPVLEVERVAKETGLEIYDKLETDSIAKEGEQGDSYYQMMKWNIE |
| | | TIHEGLSQTKES |
| 12 | Esp WT - | MVSKNNKRVFLEKTKKRVLKYSIKKLSVGVASVLVGVGLVLGTTELVQAQDEISPST |
| | E. faecium | PLETAISSVQVGDKVASGNTFQENPGYTKNYNFSDLQFSPQELTGDTLKGNTIGFEV |
| | | YGKHNIAASTKNWEIRLQLDERLAKYVEKIQVDPKKGIGSSRRTFVRINDSLGRPTN |
| | | IWKVNYIRASDGLFAGAETTDTQTAPNGVITFEKSLDEIFKEIGIDNLKTDRLMYRI |
| | | YLVSHQDDDKIVPGIDSTGYFLTDSDDFYNSLDVSENNPDQFKHGSVSAKYEEPNTQ |
| | | TKDGSGSTGANGAIILDHKLTKNYNFSYSASAKGTPWYANYKIDERLVPYVAGIQMH |
| | | MVQADKVTYDVSFESGKKVADLAIERRKDHENYGVGSITDNDLTKLIDFANASPRPV |
| | | VIRYVLQLTKPLDEILEDMKATAQVEENKPFGEDFIFDSWLSDTNKKLIQNTYGTGY |
| | | YYLQDIDGDGNPDDKEESGDTNPYIGKPELEEVYDVDTTVKGKVFIHELAGTGHKAQ |
| | | LVDKEGTVLAEKTIAPNEKDGAPISDTVEFEFTGVDSSKLIAKDELKIQIVSPGEDK |
| | | PEEGSTVIKESPKAVDKQTVVVGFKPDAKESIRNNKNLPEDAEYSWKTEPDTSNVTD |
| | | STKGIVTVKIGNRTFDVDVEFAVKASQAMENDATYVPITTTPETTVQSGKPTEDKPD |
| | | VPLAKDAFSILDVYNKDFGNASVDANTGIVTFTPAKGVGESEPITGTIPIKIVYQDG |
| | | SVGTTDLAVTVSKDIYENPGENIPAGYHKVTFTAGEGTSIESGTTVFAVKDGVSLPE |
| | | DKLPVLKAKDGYTDAKWPEEATQPITADDTEFVSSATKLDDIIENPGENIPAGYHKV |
| | | TFTAGEGTSIESGTTVFAVKDGVSLPEDKLPVLKAKDGYTDAKWPEEATQPITADDT |
| | | EFVSSATKLDDIIENPGENIPAGYHKVTFTAGEGTSIESGTTVFAVKDGVSLPEDKL |
| | | PVLKAKDGYTDAKWPEEATQPITADDTEFVSSATKLDDIIENPGENIPAGYHKVIFT |
| | | AGEGTSIESGTTVFAVKDGVSLPEDKLPVLKAKDGYTDAKWPEEATQPITADDTEFV |
| | | SSATKLDDIIENPGENIPAGYHKVIFTAGEGTSIESGTTVFAVKDGVSLPEDKLPVL |
| | | KAKDGYTDAKWPEEATQPITADDTEFVSSATKLDDKSDADKYNPEGQKVTTELNKEP |
| | | DASEGIKNKEDLPKDTKYTWKEKVDVSAAGNKKGTVVVTYSDGSSDEVEVDVTVTDN |
| | | RSDADKYEPTVEGEKVEVGGTVDLTDNVTNLPTLPEGTTVTDVTPDGTIDTNTPGNY |
| | | EGVIEVTYPDGTKDTVKVPVEVTDNRSDADKYTPMVEGEKVEVGGTVDLTDNVTNLP |
| | | TLPEGTTVTDVTPDGTIDTNTPGNYEGVIEVTYPDGTKDTVKVPVEVTDNRSDADKY |
| | | TPMVEGEKVEVGGTVDLTDNVTNLPTLPEGTTVTDVTPGGTIDTNTPGNYEGVIEVT |
| | | YPDGTKDTVKVPVEVTDNRSDADKYEPTVEGEKVEVGGTVDLTDNVTNLPTLPEGTT |
| | | VTDVTPGGTIDTNTPGNYEGVIEVTYPDGTKDTVKVPVEVTDNRSDADKYTPMVEGE |
| | | KVEVGGTVDLTDNVTNLPTLPEGTTVTDVTPDGTIDTNTPGNYEGVIEVTYPDGTKD |
| | | TVKVPVEVTDNRSDADKYNPEGQKVTTDLNKEPDASEGIKNKEDLPKGTTYTWKEKV |
| | | DVSTAGNKKGIVVVTYPDGSKEEVEVTISVEDKKAPNKPQVDPITEGDQIVTGKTEP |
| | | NAEVTVTLPNGSQYHGTADKNGNFTVKVPKLEAGTKVIVTATDESGNTSEPTNVVVS |
| | | SNEKDSEKAVSKDNKTDNQGSKQNTNRGKSSPQKQSSKAYPKTGEIDSNIFTISGGL |
| | | ILLGTLGLLGYKNRKKENE |
| 13 | Ace [30-366 | GAELSKSSIVDKVELDHTTLYQGEMTSIKVSFSDKENQKIKPGDTITLTLPDALVGM |
| | fragment] - | TENDGSPRKINLNGLGEVFIYKDHVVATFNEKVESLHNVNGHFSFGIKTLITNSSQP |
| | E. faecalis | NVIETDFGTATATQRLTIEGVTNTETGQIERDYPFFYKVGDLAGESNQVRWELNVNL |
| | | NKSDVTEDISIADRQGSGQQLNKESFTFDIVNDKETKYISLAEFEQQGYGKIDFVTD |
| | | NDENLRFYRNKARFTSFIVRYTSTITEAGQHQATFENSYDINYQLNNQDATNEKNTS |
| | | QVKNVFVEGEASGNQNVEMPTEESLDIPLETIDEWEPKTPTSEQATETSEKT |
| 14 | AdcA [25-317 | GQTTQSPKEKKELTVMTTFYPMYDFAKQVVGDEGEVELLIPAGTEPHDYEPSAKDLA |
| | fragment] - | KITDADVFVYNSKELETWVPNVIENLDTKKVSIVEASQSIQLMQGTEEEESGEEGHE |
| | E. faecalis | GHNHSHELDPHVWLDPVLAQKEVTAIRDALIKKYPEKKAVFEKNTAAYLEKLTALDK |
| | | EYQAAFAGAKNRTFVTQHAAFGYLAKQYGLTQEPIAGISPDQEPSPSRLAELKKYIK |
| | | TNNVSVIYFEASASTKVAKTLADETGVELAVLNPLESLTQKEQEAGENYVSVMKENL |
| | | AALQKSIH |
| 15 | AdcAII | GSDKTADGKEKLSIVTTFYPMYDFTKNIVGDEGDVKLLIPAGSEPHDYEPSAKDMAT |
| | [26-511 | IHDADVFVYHNENMESWVPKAAKGWKKGAPNVIKGTENMVLLPGSDEDGHDHDHEHG |
| | fragment] - | EEGHHHELDPHTWVSPHRAIQEVTNIKEQLVKLYPKKAKTFETNAEKYLTKLTALDK |
| | E. faecalis | EFQTALKDAKQKSFVTQHAAFGYLALDYGLKQVPIAGLTPEQEPTAGRLAELKKYVT |
| | | DNQIRYIYFEKNANDKIAKTLADEANVQLEVLNPLESLTQKQMDNGEDYLSVMKENL |
| | | TALKKTTDTAGKEVQPETSEKTEKTVANGYFKDSEVAERTLADYAGNWQSVYPLLKD |
| | | GTLDQVFDYKAKLKKDKTPAEYKTYYDAGYQTDVDHINITDSTIEFLVDGKPQKFTY |
| | | KAAGYKILNYAKGNRGVRFLFETDDANAGRFKYVQFSDHNIAPTKAAHFHIFFGGDS |
| | | QESLFNEMDNWPTYYPNDLSKQEIAQEMIAH |
| 16 | EbpA | ATEEVINPFAEARMAPATLRANLALPLIAPQYTTDNSGTYPTANWQPTGNQNVLNHQ |
| | [193-1032] - | GNKDGSAQWDGQTSWNGDPTNRTNSYIEYGGTGDQADYAIRKYARETTTPGLEDVYL |
| | E. faecalis | NVRGNVQKEITPLDLVLVVDWSGSMNENNRIGEVQKGVNRFVDTLADSGITNNINMG |
| | | YVGYSSDGYNNNAIQMGPFDTVKNPIKNITPSSTRGGTFTQKALRDAGDMLATPNGH |
| | | KKVIVLLTDGVPTFSYKVSRVQTEADGRFYGTQFTNRQDQPGSTSYISGSYNAPDQN |
| | | NINKRINSTFIATIGEAMALKQRGIEIHGLGIQLQSDPRANLSKQQVEDKMREMVSA |
| | | DENGDLYYESADYAPDISDYLAKKAVQISGTVVNGKVVDPIAEPFKYEPNTLSMKSV |
| | | GPVQVQTLPEVSLTGATINSNEIYLGKGQEIQIHYQVRIQTESENFKPDFWYQMNGR |
| | | TTFQPLATAPEKVDFGVPSGKAPGVKLNVKKIWEEYDQDPTSRPDNVIYEISRKQVT |
| | | DTANWQTGYIKLSKPENDTSNSWERKNVTQLSKTADESYQEVLGLPQYNNQGQAFNY |
| | | QTTRELAVPGYSQEKIDDTTWKNTKQFKPLDLKVIKNSSSGEKNLVGAVFELSGKNV |
| | | QTTLVDNKDGSYSLPKDVRLQKGERYTLTEVKAPAGHELGKKTTWQIEVSEQGKVSI |
| | | DGQEVTTTNQVIPLEIENKFSSLPIRIRKYTMQNGKQVNLAEATFALQRKNAQGSYQ |
| | | TVATQKTDTTGLSYFKISEPGEYRMVEQSGPLGYDTLAGNYEFTVDKYGKIHYAGKN |
| | | IEENAPEWTLTHQNNLKPFDLTVHKKADNQTPLKGAKERLTG |
| 17 | EfaA [24-308 | AAEKKEKLAIVTTNSILSDLVKNVGQDKIELHSIVPIGTDPHEYEPLPEDIAKASEA |
| | fragment] - | DILFFNGLNLETGGNGWENKLMKTAKKVENKDYFSTSKNVTPQYLTSAGQEQTEDPH |
| | E. faecalis | AWLDIENGIKYVENIRDVLVEKDPKNKDFYTENAKNYTEKLSKLHEEAKAKFADIPD |
| | | DKKLLVTSEGAFKYESKAYDLNAAYIWEINTESQGTPEQMTTIIDTIKKSKAPVLFV |
| | | ETSVDKRSMERVSKEVKRPIYDTLFTDSLAKEGTEGDTYYSMMNWNLTKIHDGLMSK |
| 18 | Esp [43-411 | GATGIVNAQMGEGRLANYSASGNTFQENPGYTKNYNFSDLQFNPKAITGDVLQGNTI |
| | fragment] - | DFEVYGKHNIAASTANWEIRLQLDERLAQYVEKIQVDPKKGVGNSRRTFVRINDSLG |
| | E. faecalis | RPTNIWKVNYIRANDGLFAGAETTDTQTAPNGVITFEKNLDEIFKEIGADNLKSDRL |
| | | MYRIYLVSHQDDDKIVPGIESTGYFLTDQDDFYNKLDVSENNSDQFKHGSVNTKYEE |
| | | ANIQTKDGSGSTGANGAIILDHKLTKEKNFSYSTSAKGTPWYANYKIDERLVPYVSG |
| | | IQMHMVQADKVAYNVAFESGKKVADLAIERREGHENYGMGSITDNDLTKLIDFANAS |
| | | PRPIVVRYVLQLTKPLDEILEEMKAADK |

The recombinant polypeptide antigens may be full-length wildtype (WT) polypeptide sequences from an Enterococcus species (e.g., E. faecalis, E. faecium, E. durans), such as those of SEQ ID NOs 1-12: The polypeptide antigens may additionally or alternatively be fragments of a full-length wildtype protein from an Enterococcus species. For example, in some embodiments, a polypeptide antigen of the compositions and methods described herein may be a full-length wildtype Ace E. faecalis polypeptide antigen (e.g., SEQ ID NO: 1) or a full-length wildtype E. faecium Ace polypeptide antigen (e.g., SEQ ID NO: 7). In some embodiments, the polypeptide antigen may be a fragment of a full-length wildtype polypeptide antigen (e.g., SEQ ID NO: 13, residues 30-366 of an E. faecalis Ace polypeptide). In some embodiments, a polypeptide antigen may be a fragment of any of the polypeptide antigens described herein. For instance, a fragment may be at least 10%, 15%, 20%, 25%, 30%, 35%, 40%, 45%, 50%, 55%, 60%, 65%, 70%, 75%, 80%, 85%, 90%, or 95% of the length of the polypeptide antigen (e.g., a wildtype polypeptide antigen or antigen sequence specified herein). In other words, a fragment may have at least 10%, 15%, 20%, 25%, 30%, 35%, 40%, 45%, 50%, 55%, 60%, 65%, 70%, 75%, 80%, 85%, 90%, or 95% the number of residues as the parent polypeptide antigen. In some embodiments, the polypeptide antigen fragment may be at least 10-15%, 10-20%, 10-25%, 10-30%, 10-35%, 10-40%, 10-45%, 10-50%, 10-55%, 10-65%, 10-70%, 10-75%, 10-80%, 10-85%, 10-90%, 10-95%, 15-20%, 15-25%, 15-30%, 15-35%, 15-40%, 15-45%, 15-50%, 15-55%, 15-65%, 15-70%, 15-75%, 15-80%, 15-85%, 15-90%, 15-95%, 20-25%, 20-30%, 20-35%, 20-40%, 20-45%, 20-50%, 20-55%, 20-65%, 20-70%, 20-75%, 20-80%, 20-85%, 20-90%, 20-95%, 25-30%, 25-35%, 25-40%, 25-45%, 25-50%, 25-55%, 25-65%, 25-70%, 25-75%, 25-80%, 25-85%, 25-90%, 25-95%, 30-35%, 30-40%, 30-45%, 30-50%, 30-55%, 30-65%, 30-70%, 30-75%, 30-80%, 30-85%, 30-90%, 30-95%, 35-40%, 35-45%, 35-50%, 35-55%, 35-65%, 35-70%, 35-75%, 35-80%, 35-85%, 35-90%, 35-95%, 40-45%, 40-50%, 40-55%, 40-65%, 40-70%, 40-75%, 40-80%, 40-85%, 40-90%, 40-95%, 45-50%, 45-55%, 45-65%, 45-70%, 45-75%, 45-80%, 45-85%, 45-90%, 45-95%, 50-55%, 50-65%, 50-70%, 50-75%, 50-80%, 50-85%, 50-90%, 50-95%, 55-65%, 55-70%, 55-75%, 55-80%, 55-85%, 55-90%, 55-95%, 65-70%, 65-75%, 65-80%, 65-85%, 65-90%, 65-95%, 65-75%, 65-80%, 65-85%, 65-90%, 65-95%, 70-75%, 70-80%, 70-85%, 70-90%, 70-95%, 75-75%, 75-80%, 75-85%, 75-90%, 75-95%, 80-85%, 80-90%, 80-95%, 85-90%, 85-95%, or 85-95% of the length of the polypeptide antigen or antigen sequence specified herein, and/or at least. Generally, useful fragments are antigenic, e.g., the fragment will be of sufficient length to provoke a protective immune response (e.g., such as measured by the methods, including animal models, described in this specification, including the examples).

The immunogenic compositions described herein comprise at least one recombinant Enterococcus (e.g., E. faecalis, E. faecium, E. durans) polypeptide antigen or a fragment thereof. In some embodiments of such immunogenic compositions, the at least one recombinant E. faecalis polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen. In some embodiments of such immunogenic compositions, the at least one recombinant E. faecium polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen. In some embodiments of such immunogenic compositions, the at least one recombinant E. durans polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen.

In some embodiments, the at least one recombinant Enterococcus polypeptide antigen is an Ace polypeptide antigen or a fragment thereof. The Ace polypeptide antigen may, for instance, comprise or consist of the amino acid sequence of SEQ ID NOs: 1, 7, or 13. In some embodiments, the Ace polypeptide antigen comprises or consists of an amino acid sequence that is at least 85%, 90%, 95%, 96%, 97%, 98%, or 99% identical to any one of SEQ ID NOs: 1, 7, or 13. In some embodiments, the Ace polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 1, 7, or 13.

“Percent identity” (e.g., “90% identical to”) in the context of the polypeptide sequences described herein, refers to two or more sequences that are the same or have a specified percentage of amino acid residues that are the same, when compared and aligned for maximum correspondence over a given length (comparison window), as measured using a sequence comparison algorithm (e.g., BLASTP or the Smith-Waterman homology search algorithm). In the present context, the percent sequence homology may be determined over the full-length of the polypeptide or just a portion. One method for calculating percent sequence homology is the BLASTP program having its defaults set at a wordlength (W) of 3, an expectation (E) of 10, and the BLOSUM62 scoring matrix; see, e.g., Henikoff et al. (1989) Proc. Natl. Acad. Sci. USA 89: 15915. Exemplary determination of sequence alignment and % sequence identity employs the BESTFIT or GAP programs in the GCG Wisconsin Software package (Accelrys, Madison Wis.), using the default parameters provided. If these preferred methods of calculating sequence identity give differing amounts, the method giving the higher sequence identity controls.

In some embodiments, the at least one recombinant Enterococcus polypeptide antigen is an AdcA polypeptide antigen or a fragment thereof. The AdcA polypeptide antigen may, for instance, comprise or consist of the amino acid sequence of SEQ ID NOs: 2, 8, or 14. In some embodiments, the AdcA polypeptide antigen comprises or consists of an amino acid sequence that is at least 85%, 90%, 95%, 96%, 97%, 98%, or 99% identical to any one of SEQ ID NOs: 2, 8, or 14. In some embodiments, the AdcA polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 2, 8, or 14.

In some embodiments, the at least one recombinant Enterococcus polypeptide antigen is an AdcAII polypeptide antigen or a fragment thereof. The AdcAII polypeptide antigen may, for instance, comprise or consist of the amino acid sequence of SEQ ID NOs: 3, 9, or 15. In some embodiments, the AdcAII polypeptide antigen comprises or consists of an amino acid sequence that is at least 85%, 90%, 95%, 96%, 97%, 98%, or 99% identical to any one of SEQ ID NOs: 3, 9, or 15. In some embodiments, the AdcAII polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 3, 9, or 15.

In some embodiments, the at least one recombinant Enterococcus polypeptide antigen is an EbpA polypeptide antigen or a fragment thereof. The EbpA polypeptide antigen may, for instance, comprise or consist of the amino acid sequence of SEQ ID NOs: 4, 15, or 16. In some embodiments, the EbpA polypeptide antigen comprises or consists of an amino acid sequence that is at least 85%, 90%, 95%, 96%, 97%, 98%, or 99% identical to any one of SEQ ID NOs: 4, 15, or 16. In some embodiments, the EbpA polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 4, 15, or 16.

In some embodiments, the at least one recombinant Enterococcus polypeptide antigen is an EfaA polypeptide antigen or a fragment thereof. The EfaA polypeptide antigen may, for instance, comprise or consist of the amino acid sequence of SEQ ID NOs: 5, 11, or 17. In some embodiments, the EfaA polypeptide antigen comprises or consists of an amino acid sequence that is at least 85%, 90%, 95%, 96%, 97%, 98%, or 99% identical to any one of SEQ ID NOs: 5, 11, or 17. In some embodiments, the EfaA polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 5, 11, or 17.

In some embodiments, the at least one recombinant Enterococcus polypeptide antigen is an Esp polypeptide antigen or a fragment thereof. The Esp polypeptide antigen may, for instance, comprise or consist of the amino acid sequence of SEQ ID NOs: 6, 12, or 18. In some embodiments, the Esp polypeptide antigen comprises or consists of an amino acid sequence that is at least 85%, 90%, 95%, 96%, 97%, 98%, or 99% identical to any one of SEQ ID NOs: 6, 12, or 18. In some embodiments, the Esp polypeptide antigen comprises or consists of an amino acid sequence that is at least 15%, 15%, 20%, 25%, 30%, 35%, 40%, 45%, 50%, 55%, 60%, 65%, 70%, 75%, 80%, 85%, 90%, or 95% of the length of any one of SEQ ID NOs: 6, 12, or 18.

The immunogenic compositions described herein may comprise or consist of any number of recombinant Enterococcus polypeptide antigens. For instance, an immunogenic composition may comprise or consist of 1, 2, 3, 4, 5, 6, or more Enterococcus polypeptide antigens. An immunogenic composition may, for example, comprise or consist of between 1 and 6; 1 and 5; 1 and 4; 1 and 3; 1 and 2; 2 and 6; 2 and 5; 2 and 4; 2 and 3; 3 and 6; 3 and 5; 3 and 4; 4 and 6; 4 and 5; or 5 and 6 polypeptide antigens. In some embodiments, the immunogenic compositions may comprise polypeptide antigens of different Enterococcus proteins (e.g., a wildtype Ace polypeptide antigen and a wildtype AdcA polypeptide antigen). Additionally, or alternatively, the immunogenic compositions may comprise multiple polypeptide antigens of the same Enterococcus protein (e.g., two different fragments of an AdcA polypeptide antigen). In some embodiments, the 1, 2, 3, 4, 5, 6, or more of the Enterococcus polypeptide antigens are fragments of the polypeptide antigens.

In some embodiments, the immunogenic compositions may comprise more than one recombinant polypeptide antigen, or fragment thereof, from the same Enterococcus species (e.g., two or more polypeptide antigens from E. faecalis). In certain embodiments, the immunogenic compositions may comprise more than one polypeptide antigen, or fragment thereof, from different Enterococcus species (e.g., at least one polypeptide antigen from E. faecalis and at least one polypeptide antigen from E. faecium; at least one polypeptide antigen from E. faecalis and at least one polypeptide antigen from E. faecium; at least one polypeptide antigen from E. faecalis and at least one polypeptide antigen from E. durans; at least one polypeptide antigen from E. faecium and at least one polypeptide antigen from E. durans; at least one polypeptide from each of E. Faceium, E. faecalis, and E. durans).

In some embodiments, the immunogenic compositions described herein comprise at least one recombinant E. faecalis polypeptide antigen or a fragment thereof. For instance, an immunogenic composition may comprise a first recombinant E. faecalis polypeptide antigen and a second recombinant E. faecalis polypeptide. An immunogenic composition may comprise a third, fourth, fifth, or sixth E. faecalis polypeptide. Thus, in certain embodiments, an immunogenic composition may comprise a first recombinant E. faecalis polypeptide antigen, a second recombinant E. faecalis polypeptide, and a third recombinant E. faecalis polypeptide. In certain embodiments, an immunogenic composition may comprise a first recombinant E. faecalis polypeptide antigen, a second recombinant E. faecalis polypeptide, a third recombinant E. faecalis polypeptide, and a fourth recombinant E. faecalis polypeptide. In certain embodiments, an immunogenic composition may comprise a first recombinant E. faecalis polypeptide antigen, a second recombinant E. faecalis polypeptide, a third recombinant E. faecalis polypeptide, a fourth recombinant E. faecalis polypeptide, and a fifth recombinant E. faecalis polypeptide. In some embodiments, an immunogenic composition may comprise a first recombinant E. faecalis polypeptide antigen, a second recombinant E. faecalis polypeptide, a third recombinant E. faecalis polypeptide, a fourth recombinant E. faecalis polypeptide, a fifth recombinant E. faecalis polypeptide, and a sixth recombinant E. faecalis polypeptide.

In a certain embodiments, the immunogenic compositions described herein comprise at least one recombinant E. faecium polypeptide antigen or a fragment thereof. For instance, an immunogenic composition may comprise a first recombinant E. faecium polypeptide antigen and a second recombinant E. faecium polypeptide. An immunogenic composition may comprise a third, fourth, fifth, or sixth E. faecium polypeptide. Thus, in certain embodiments, an immunogenic composition may comprise a first recombinant E. faecium polypeptide antigen, a second recombinant E. faecium polypeptide, and a third recombinant E. faecium polypeptide. In certain embodiments, an immunogenic composition may comprise a first recombinant E. faecium polypeptide antigen, a second recombinant E. faecium polypeptide, a third recombinant E. faecium polypeptide, and a fourth recombinant E. faecium polypeptide. In certain embodiments, an immunogenic composition may comprise a first recombinant E. faecium polypeptide antigen, a second recombinant E. faecium polypeptide, a third recombinant E. faecium polypeptide, a fourth recombinant E. faecium polypeptide, and a fifth recombinant E. faecium polypeptide. In some embodiments, an immunogenic composition may comprise a first recombinant E. faecium polypeptide antigen, a second recombinant E. faecium polypeptide, a third recombinant E. faecium polypeptide, a fourth recombinant E. faecium polypeptide, a fifth recombinant E. faecium polypeptide, and a sixth recombinant E. faecium polypeptide.

In a certain embodiments, the immunogenic compositions described herein comprise at least one recombinant E. durans polypeptide antigen or a fragment thereof. For instance, an immunogenic composition may comprise a first recombinant E. durans polypeptide antigen and a second recombinant E. durans polypeptide. An immunogenic composition may comprise a third, fourth, fifth, or sixth E. durans polypeptide. Thus, in certain embodiments, an immunogenic composition may comprise a first recombinant E. durans polypeptide antigen, a second recombinant E. durans polypeptide, and a third recombinant E. durans polypeptide. In certain embodiments, an immunogenic composition may comprise a first recombinant E. durans polypeptide antigen, a second recombinant E. durans polypeptide, a third recombinant E. durans polypeptide, and a fourth recombinant E. durans polypeptide. In certain embodiments, an immunogenic composition may comprise a first recombinant E. durans polypeptide antigen, a second recombinant E. durans polypeptide, a third recombinant E. durans polypeptide, a fourth recombinant E. durans polypeptide, and a fifth recombinant E. durans polypeptide. In some embodiments, an immunogenic composition may comprise a first recombinant E. durans polypeptide antigen, a second recombinant E. durans polypeptide, a third recombinant E. durans polypeptide, a fourth recombinant E. durans polypeptide, a fifth recombinant E. durans polypeptide, and a sixth recombinant E. durans polypeptide.

For clarity, it should be noted that the numbering designations used herein (e.g., first, second, third, etc. recombinant polypeptide antigen) are interchangeable within a given composition. For instance, in an immunogenic composition comprising a first, second, and third recombinant polypeptide antigen, where the three recombinant polypeptide antigens are AdcAII, EfaA, and EbpA, each of these three recombinant polypeptide antigens could be called the first polypeptide antigen, where the remaining two are the second and third (interchangeably).

Additionally, or alternatively, an immunogenic composition described herein may not comprise a second recombinant Enterococcus polypeptide antigen. For instance, in some embodiments, an immunogenic composition does not comprise a second recombinant E. faecalis polypeptide antigen (e.g., the immunogenic composition may comprise only one recombinant E. faecalis polypeptide antigen. In certain embodiments, an immunogenic composition does not comprise a third recombinant polypeptide antigen (e.g., the immunogenic composition may comprise no more than 1 or 2 polypeptide antigens). In certain embodiments, an immunogenic composition does not comprise a fourth recombinant polypeptide antigen (e.g., the immunogenic composition may comprise no more than 1, 2, or 3 polypeptide antigens). In certain embodiments, an immunogenic composition does not comprise a fifth recombinant polypeptide antigen (e.g., the immunogenic composition may comprise no more than 1, 2, 3, or 4 polypeptide antigens). In some embodiments, an immunogenic composition does not comprise a sixth recombinant polypeptide antigen (e.g., the immunogenic composition may comprise no more than 1, 2, 3, 4, or 5 polypeptide antigens). In some embodiments, one or more of the at least one polypeptide antigen is a fragment of the polypeptide antigen.

Different combinations of the recombinant polypeptide antigens may have different immunogenic properties, allowing for the activity of the final immunogenic compositions to be fine-tuned. In some embodiments, an immunogenic composition comprises a first and a second recombinant E. faecalis polypeptide antigen. In some embodiments, the first polypeptide antigen is an AdcAII polypeptide antigen and the second recombinant E. faecalis polypeptide antigen is an EfaA polypeptide antigen. In some embodiments, the AdcAII polypeptide antigen comprises or consists of the amino acid sequence of SEQ ID NO: 3 or SEQ ID NO: 15; and the EfaA polypeptide antigen comprises or consists of the amino acid sequence of SEQ ID NO: 5 or SEQ ID NO: 17. In certain embodiments, the AdcAII polypeptide antigen comprises or consists of the amino acid sequence of SEQ ID NO: 15, and the EfaA polypeptide antigen comprises or consists of the amino acid sequence of SEQ ID NO: 17. In some embodiments, one or more of the polypeptide antigens is a fragment of the polypeptide antigen.

In some embodiments, an immunogenic composition comprises a first and a second recombinant E. faecium polypeptide antigen or fragment thereof. In some embodiments, the first polypeptide antigen is an AdcAII polypeptide antigen and the second recombinant E. faecium polypeptide antigen is an EfaA polypeptide antigen. In some embodiments, the AdcAII polypeptide antigen comprises or consists of the amino acid sequence of SEQ ID NO: 9; and the EfaA polypeptide antigen comprises or consists of the amino acid sequence of SEQ ID NO: 11. In certain embodiments, the AdcAII polypeptide antigen comprises or consists of the amino acid sequence of SEQ ID NO: 9, and the EfaA polypeptide antigen comprises or consists of the amino acid sequence of SEQ ID NO: 11. In some embodiments, one or more of the polypeptide antigens is a fragment of the polypeptide antigen.

In some instances, an immunogenic composition comprises a first, second, and third recombinant E. faecalis polypeptide antigen. In some embodiments, the first recombinant E. faecalis polypeptide antigen is an AdcAII polypeptide antigen, the second recombinant E. faecalis polypeptide antigen is an Esp polypeptide antigen, and the third recombinant E. faecalis polypeptide antigen is an Ace polypeptide antigen. In some embodiments, the AdcAII polypeptide antigen comprises an amino acid sequence that is at least 95% identical to SEQ ID NO: 3 or SEQ ID NO: 15; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13. In certain embodiments, the AdcAII polypeptide antigen comprises or consists of the amino acid sequence of SEQ ID NO: 15, the Esp polypeptide antigen comprises or consists of the amino acid sequence of SEQ ID NO: 18, and the Ace polypeptide antigen comprises or consists of the amino acid sequence of SEQ ID NO: 13. In some embodiments, one or more of the polypeptide antigens is a fragment of the polypeptide antigen.

In some instances, an immunogenic composition comprises a first, second, and third recombinant E. faecium polypeptide antigen. In some embodiments, the first recombinant E. faecium polypeptide antigen is an AdcAII polypeptide antigen, the second recombinant E. faecium polypeptide antigen is an Esp polypeptide antigen, and the third recombinant E. faecium polypeptide antigen is an Ace polypeptide antigen. In some embodiments, the AdcAII polypeptide antigen comprises an amino acid sequence that is at least 95% identical to SEQ ID NO: 9; the Esp polypeptide antigen comprises an amino acid sequence that is at least 95% identical to SEQ ID NO: 12; and the Ace polypeptide antigen comprises an amino acid sequence that is at least 95% identical to SEQ ID NO: 7. In certain embodiments, the AdcAII polypeptide antigen comprises or consists of the amino acid sequence of SEQ ID NO: 9, the Esp polypeptide antigen comprises or consists of the amino acid sequence of SEQ ID NO: 12, and the Ace polypeptide antigen comprises or consists of the amino acid sequence of SEQ ID NO: 7. In some embodiments, one or more of the polypeptide antigens is a fragment of the polypeptide antigen.

In some embodiments, the first recombinant E. faecalis polypeptide antigen is an AdcAII polypeptide antigen, the second recombinant E. faecalis polypeptide antigen is an EfaA polypeptide antigen, and the third recombinant E. faecalis polypeptide antigen is an EbpA polypeptide antigen. In some embodiments, the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16. In certain embodiments, the AdcAII polypeptide antigen comprises or consists of the amino acid sequence of SEQ ID NO: 15, the EfaA polypeptide antigen comprises or consists of the amino acid sequence of SEQ ID NO: 17, and the EbpA polypeptide antigen comprises or consists of the amino acid sequence of SEQ ID NO: 16. In some embodiments, one or more of the polypeptide antigens is a fragment of the polypeptide antigen.

In some embodiments, the first recombinant E. faecium polypeptide antigen is an AdcAII polypeptide antigen, the second recombinant E. faecium polypeptide antigen is an EfaA polypeptide antigen, and the third recombinant E. faecium polypeptide antigen is an EbpA polypeptide antigen. In some embodiments, the AdcAII polypeptide antigen comprises or consists of the amino acid sequence of SEQ ID NO: 9; the EfaA polypeptide antigen comprises or consists of the amino acid sequence of SEQ ID NO: 11; and the EbpA polypeptide antigen comprises or consists of the amino acid sequence of SEQ ID NO: 10 In some embodiments, one or more of the polypeptide antigens is a fragment of the polypeptide antigen.

In some embodiments, the first recombinant E. faecalis polypeptide antigen is an AdcAII polypeptide antigen, the second recombinant E. faecalis polypeptide antigen is an Ace polypeptide antigen, and the third recombinant E. faecalis polypeptide antigen is an EbpA polypeptide antigen. In some embodiments, the AdcAII polypeptide antigen comprises or consists of the amino acid sequence of SEQ ID NO: 3 or SEQ ID NO: 15; the Ace polypeptide antigen comprises or consists of the amino acid sequence of SEQ ID NO: 1 or SEQ ID NO: 13; and the EbpA polypeptide antigen comprises or consists of the amino acid sequence of SEQ ID NO: 4 or SEQ ID NO: 16. In certain embodiments, the AdcAII polypeptide antigen comprises or consists of the amino acid sequence of SEQ ID NO: 15, the Ace polypeptide antigen comprises or consists of the amino acid sequence of SEQ ID NO: 13, and the EbpA polypeptide antigen comprises or consists of the amino acid sequence of SEQ ID NO: 16. In some embodiments, one or more of the polypeptide antigens is a fragment of the polypeptide antigen.

In some embodiments, the first recombinant E. faecium polypeptide antigen is an AdcAII polypeptide antigen, the second recombinant E. faecium polypeptide antigen is an Ace polypeptide antigen, and the third recombinant E. faecium polypeptide antigen is an EbpA polypeptide antigen. In some embodiments, the AdcAII polypeptide antigen comprises or consists of the amino acid sequence of SEQ ID NO: 9; the Ace polypeptide antigen comprises or consists of the amino acid sequence of SEQ ID NO: 7; and the EbpA polypeptide antigen comprises or consists of the amino acid sequence of SEQ ID NO: 10. In some embodiments, one or more of the polypeptide antigens is a fragment of the polypeptide antigen.

In some embodiments, the first recombinant E. faecalis polypeptide antigen is an AdcA polypeptide antigen, the second recombinant E. faecalis polypeptide antigen is an EfaA polypeptide antigen, and the third recombinant E. faecalis polypeptide antigen is an EbpA polypeptide antigen. In some embodiments, the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16. In certain embodiments, the AdcA polypeptide antigen comprises or consists of the amino acid sequence of SEQ ID NO: 14, the EfaA polypeptide antigen comprises or consists of the amino acid sequence of SEQ ID NO: 17, and the EbpA polypeptide antigen comprises or consists of the amino acid sequence of SEQ ID NO: 16. In some embodiments, one or more of the polypeptide antigens is a fragment of the polypeptide antigen.

In some embodiments, the first recombinant E. faecium polypeptide antigen is an AdcA polypeptide antigen, the second recombinant E. faecium polypeptide antigen is an EfaA polypeptide antigen, and the third recombinant E. faecium polypeptide antigen is an EbpA polypeptide antigen. In some embodiments, the AdcAII polypeptide antigen comprises or consists of the amino acid sequence of SEQ ID NO: 9; the EfaA polypeptide antigen comprises or consists of the amino acid sequence of SEQ ID NO: 11; and the EbpA polypeptide antigen comprises or consists of the amino acid sequence of SEQ ID NO: 15. In some embodiments, one or more of the polypeptide antigens is a fragment of the polypeptide antigen.

In some instances, an immunogenic composition comprises a first, second, third, and fourth recombinant E. faecalis polypeptide antigen. In some instances, an immunogenic composition comprises a first, second, third, and fourth recombinant E. faecium polypeptide antigen. In some embodiments, one or more of the polypeptide antigens is a fragment of the polypeptide antigen.

In some instance, an immunogenic composition comprises a first, second, third, fourth, and fifth recombinant E. faecalis polypeptide antigen In some embodiments, the first recombinant E. faecalis polypeptide antigen is an AdcA polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen, and the fifth recombinant E. faecalis polypeptide antigen is an Ace polypeptide antigen. In some embodiments, the AdcA polypeptide antigen comprises or consists of the amino acid sequence of SEQ ID NO: 2 or SEQ ID NO: 14; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13. In certain embodiments, the AdcA polypeptide antigen comprises or consists of the amino acid sequence of SEQ ID NO: 14, the EfaA polypeptide antigen comprises or consists of the amino acid sequence of SEQ ID NO: 17, the EbpA polypeptide antigen comprises or consists of the amino acid sequence of SEQ ID NO: 16, the Esp polypeptide antigen comprises or consists of the amino acids sequence of SEQ ID NO: 18, and the Ace polypeptide antigen comprises or consists of the amino acid sequence of SEQ ID NO: 13. In some embodiments, one or more of the polypeptide antigens is a fragment of the polypeptide antigen.

In some instance, an immunogenic composition comprises a first, second, third, fourth, and fifth recombinant E. faecium polypeptide antigen In some embodiments, the first recombinant E. faecium polypeptide antigen is an AdcA polypeptide antigen, the second recombinant E. faecium polypeptide antigen is an EfaA polypeptide antigen, the third recombinant E. faecium polypeptide antigen is an EbpA polypeptide antigen, the fourth recombinant E. faecium polypeptide antigen is an Esp polypeptide antigen, and the fifth recombinant E. faecium polypeptide antigen is an Ace polypeptide antigen. In some embodiments, the AdcA polypeptide antigen comprises or consists of the amino acid sequence of SEQ ID NO: 8; the EfaA polypeptide antigen comprises or consists of the amino acid sequence of SEQ ID NO: 11; the EbpA polypeptide antigen comprises or consists of the amino acid sequence of SEQ ID NO: 10; the Esp polypeptide antigen comprises or consists of the amino acid sequence of SEQ ID NO: 12; and the Ace polypeptide antigen comprises or consists of the amino acid sequence of SEQ ID NO: 7. In some embodiments, one or more of the polypeptide antigens is a fragment of the polypeptide antigen.

In some embodiments, the first recombinant E. faecalis polypeptide antigen is an AdcAII polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen; and the fifth recombinant E. faecalis polypeptide antigen is an Ace polypeptide antigen. In some embodiments, the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13. In certain embodiments, the AdcAII polypeptide antigen comprises or consists of the amino acid sequence of SEQ ID NO: 15, the EfaA polypeptide antigen comprises or consists of the amino acid sequence of SEQ ID NO: 17, the EbpA polypeptide antigen comprises or consists of the amino acid sequence of SEQ ID NO: 16, the Esp polypeptide antigen comprises or consists of the amino acids sequence of SEQ ID NO: 18, and the Ace polypeptide antigen comprises or consists of the amino acid sequence of SEQ ID NO: 13. In some embodiments, one or more of the polypeptide antigens is a fragment of the polypeptide antigen.

In some embodiments, the first recombinant E. faecium polypeptide antigen is an AdcAII polypeptide antigen, the second recombinant E. faecium polypeptide antigen is an EfaA polypeptide antigen, the third recombinant E. faecium polypeptide antigen is an EbpA polypeptide antigen, the fourth recombinant E. faecium polypeptide antigen is an Esp polypeptide antigen; and the fifth recombinant E. faecium polypeptide antigen is an Ace polypeptide antigen. In some embodiments, the AdcAII polypeptide antigen comprises or consists of the amino acid sequence of SEQ ID NO: 9; the EfaA polypeptide antigen comprises or consists of the amino acid sequence of SEQ ID NO: 11; the EbpA polypeptide antigen comprises or consists of the amino acid sequence of SEQ ID NO: 10; the Esp polypeptide antigen comprises or consists of the amino acid sequence of SEQ ID NO: 12; and the Ace polypeptide antigen comprises or consists of the amino acid sequence of SEQ ID NO: 7. In some embodiments, one or more of the polypeptide antigens is a fragment of the polypeptide antigen.

The present disclosure provides immunogenic compositions comprising additional adjuvant and excipients. In some embodiments, an immunogenic composition comprises one or more adjuvants to potentiate the immune response to one or more of the polypeptide antigens described herein. Suitable vaccine adjuvants include, by way of example only, aluminum salts, saponin, bacteria-related adjuvants, TLR-agonists, and/or combinations thereof. Examples of aluminum salts include aluminum phosphate, aluminum hydroxide, and aluminum sulfate (e.g., alum). In some embodiments, saponins include saponin Quil A and the Quil A-derived saponin QS-21, as well as immune stimulating complexes (ISCOMs) formed upon admixture of cholesterol, phospholipid, and a saponin. Bacteria-related adjuvants may include, without limitation, cell wall peptidoglycans and lipopolysaccharides derived from Gram negative bacteria such as Mycobacterium spp., Corynebacterium parvum, C. granulosum, Bordetella pertussis, and Neisseria meningitis, such as Lipid A, monophosphoryl Lipid A (MPLA), other Lipid A derivatives and mimetics (e.g., RC529), enterobacterial lipopolysaccharide (“LPS”), TLR4 ligands, and trehalose dimycolate (“TDM”). In some embodiments, TLR-agonist adjuvants may include BCG, PAM3CSK4, MALP-2, zymosan, poly I: C, LPS, flagellin, imiquimod, resiquimod, and CpG ODN.

Excipients may be added to optimize, for instance, the stability or delivery of the immunogenic compositions described herein. The excipients are immunologically and pharmacologically inert components that are “pharmaceutically acceptable.” A “pharmaceutically acceptable” component herein is one that (1) can be included in a immunogenic composition administered to a subject without causing significant unwanted biological effects or interacting in a deleterious manner with any of the other components of the formulation; and (2) meets the criteria set out in the Inactive Ingredient prepared by the U.S. Food and Drug Administration, and, preferably, has also been designated “Generally Regarded as Safe” (“GRAS”). The type of excipient or excipients incorporated into the immunogenic compositions described herein will depend, in part, on the selected mode of administration and the particular formulation type or dosage form, e.g., injectable liquid formulations, intranasal spray formulations, or the like; modes of administration and corresponding formulations are discussed infra. In general, however, inert components that can be advantageously incorporated into the immunogenic compositions described herein include, without limitation, vehicles, solubilizers, emulsifiers, stabilizers, preservatives, isotonicity agents, buffer systems, dispersants, diluents, viscosity modifiers, absorption enhancers, and combinations thereof. A thorough discussion of pharmaceutically acceptable inert additives is available in Gennaro (2000) Remington: The Science and Practice of Pharmacy, 20th Ed., ISBN: 0683306472.

In some embodiments, the immunogenic compositions described herein are provided as a sterile formulation for administration to a subject, e.g., as a suspension, solution or in lyophilized form to be rehydrated prior to use.

### Therapeutic Methods

As discussed herein, the immunogenic compositions of the present disclosure may be suitable for both therapeutic and prophylactic uses against Enterococcus infection. In some embodiments, the present disclosure provides a method of inducing a protective immune response against an Enterococcus bacterium (e.g., E. faecalis, E. faecium, E. durans) in a subject comprising administering an immunogenic composition described herein to the subject. In certain embodiments are provided the use of the immunogenic compositions described herein for inducing a protective immune response against an Enterococcus bacterium in a subject. In some embodiments, provided herein are the use of the immunogenic compositions described herein in the manufacture of a medicament for inducing a protective immune response against an Enterococcus bacterium in a subject.

As used herein, the term “protective immune response” encompasses eliciting an antibody response (e.g., anti-Enterococcus) in a subject. Antibody titers generated after administration of the immunogenic compositions described herein can be determined by means known in the art, for example by ELISA assays of serum samples derived from immunized subjects (See Example 4 herein). In some embodiments, the immunogenic compositions described herein elicit antibody responses in treated subjects, wherein the antibodies generated bind to one or multiple (i.e., two or more) Enterococcus peptides. For instance, the immunogenic compositions described herein comprising polypeptides from one Enterococcus species (e.g., E. faecalis) may generate antibodies in a subject that are cross-reactive with the corresponding peptides present in a different Enterococcus species (e.g., E. faecium, E. durans, or any combination of E. faecalis, E. faecium, and E. durans). This enables the vaccines described herein to protect against, or treat, infection of multiple pathogenic Enterococcus species. In some embodiments, the immunogenic compositions described herein do not elicit antibody responses against human proteins or tissue.

Herein, the term “subject” refers to a mammal. In some embodiments, the subject is a mouse, a rat, a dog, a cat, a guinea pig, a rabbit, a sheep, a horse, a cow, a non-human primate, or a human. In some embodiments, the subject is a human.

As stated above, provided herein are methods and uses of the immunogenic compositions for inducing a protective immune response against an Enterococcus bacterium in a subject. The methods and uses may involve administration of the immunogenic composition therapeutically, i.e., to treat a subject suffering from Enterococcus-related root canal failure, endocarditis, bacteremia, urinary tract infections, prostatitis, intraabdominal infection, cellulitis, dysbiotic gastrointestinal tract, prosthetic joint infection, or wound infections. The methods involve administration of the immunogenic composition prophylactically, meaning that, for example, the method reduces the risk of Enterococcus-related root canal failure, endocarditis, bacteremia, urinary tract infections, prostatitis, intraabdominal infection, cellulitis, dysbiotic gastrointestinal tract, prosthetic joint infection, or wound infections developing in a subject. When the immunogenic composition is used prophylactically, the subject may be predisposed to an Enterococcus infection as a result of any number of risk factors, including, but not limited to location (e.g., hospital stay, veterinary hospital stay) or predisposition (e.g., previous Enterococcus infection, weakened immune system). In some embodiments, the Enterococcus bacterium is E. faecalis, E. faecium, or E. durans. In some embodiments, the Enterococcus bacterium is E. faecalis.

Provided herein are also methods, and uses of an immunogenic composition, for treating a disorder in a subject in need thereof. As described above for prophylactic treatment, the subject in need thereof may be suffering from, for instance, root canal failure, endocarditis, bacteremia, urinary tract infections, prostatitis, intraabdominal infection, cellulitis, dysbiotic gastrointestinal tract, prosthetic joint infection, or wound infections. In some embodiments, the Enterococcus bacterium is E. faecalis or E. faecium. In some embodiments, the Enterococcus bacterium is E. faecalis. In some embodiments, the Enterococcus bacterium is E. faecium. In some embodiments, the Enterococcus bacterium is E. durans.

In any of the methods, the immunogenic composition for use, or the use of an immunogenic composition described herein, the subject may, in some embodiments, be at risk for, or is experiencing one or more of root canal failure, endocarditis, bacteremia, urinary tract infections, prostatitis, intraabdominal infection, cellulitis, dysbiotic gastrointestinal tract, prosthetic joint infection, or wound infections. In some embodiments of the methods, immunogenic compositions for use, or the use of immunogenic compositions herein, the risk of one or more of root canal failure, endocarditis, bacteremia, urinary tract infections, prostatitis, intraabdominal infection, cellulitis, dysbiotic gastrointestinal tract, prosthetic joint infection, or wound infections in a subject is reduced compared to before administration of the immunogenic composition.

The mode of administration largely dictates the type of formulation or dosage form that comprises the immunogenic composition. Compositions formulated for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain excipients such as solubilizers, emulsifiers, stabilizers, preservatives, isotonicity agents, buffer systems, dispersants, diluents, viscosity modifiers, absorption enhancers, and combinations thereof. Injectable formulations are rendered sterile by incorporation of a sterilizing agent, filtration through a bacteria-retaining filter, irradiation, or heat. They can also be manufactured using a sterile injectable medium. The immunogenic composition or individual components thereof may also be in dried, e.g., lyophilized, form that may be rehydrated with a suitable vehicle immediately prior to administration via injection.

The immunogenic compositions described herein are administered to a subject within the context of an appropriate dosage regimen. The composition may be administered once, or two or more times spaced out over an extended time period. For example, an initial, “prime” dose may be followed by at least one “boost” dose. The time interval between the prime and the subsequent boost dose, and between boost doses, is usually in the range of about 2 to about 24 weeks, more typically in the range of about 2 to 12 weeks, such as 2 to 8 weeks, 3-6 weeks, etc. Regardless of the mode of administration, e.g., intramuscular injection, intranasal administration, or the like, the volume of a single dose of the vaccine will generally be in the range of about 1 μL to about 500 μL, typically in the range of about 1 μL to about 250 μL, about 2.5 μL to about 200 μL, or about 5 μL to about 150 μL. It will be appreciated that the concentration of total antigen in the immunogenic composition corresponds to an immunologically effective dose of the composition per unit volume, working from the aforementioned dose volume guidelines.

For ease of use, the immunogenic composition of the invention can be incorporated into a packaged product, or “kit,” including instructions for self-administration or administration by a medical practitioner. The kit includes a sealed container housing a dose of the vaccine formulation, typically a “unit dose” appropriate for a single dosage event that is immunologically effective. The vaccine may be in liquid form and thus ready to administer as an injection or the like, or it may be in another form that requires the user to perform a preparation process prior to administration, e.g., hydration of a lyophilized formulation, activation of an inert component, or the like. The kit may also include two or more sealed containers with the prime dose in a first container and a boost dose in one or more additional containers, or an Enterococcus vaccine formulation in a first container and a vaccine directed against another infection, which may or may not be related to the Enterococcus infection, in another container.

It is to be understood that while the invention has been described in conjunction with a number of specific embodiments, the foregoing description as well as the experimental section that follows are intended to illustrate and not limit the scope of the invention. In this regard, no attempt is made to show structural details of the invention in more detail than is necessary for the fundamental understanding of the invention, the description taken with the drawings and/or examples making apparent to those skilled in the art how the invention may be embodied in practice. This disclosure includes all modifications and equivalents of the subject matter recited in the claims appended hereto as permitted by applicable law. Moreover, any combination of the elements of the invention described herein are encompassed by the disclosure unless otherwise indicated herein or clearly contradicted by context.

### Enumerated Embodiments

Embodiment I-1. An immunogenic composition comprising at least one recombinant Enterococcus faecalis (E. faecalis) polypeptide antigen or a fragment thereof.

Embodiment I-2. The immunogenic composition of embodiment I-1, wherein the at least one recombinant E. faecalis polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen.

Embodiment I-3. The immunogenic composition of embodiment I-1 or I-2, wherein the at least one recombinant E. faecalis polypeptide antigen comprises a first recombinant E. faecalis polypeptide antigen and a second recombinant E. faecalis polypeptide antigen.

Embodiment I-4. The immunogenic composition of embodiment I-3, wherein the first recombinant E. faecalis polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-5. The immunogenic composition of any one of embodiments I-1 to I-4, wherein the at least one recombinant E. faecalis polypeptide antigen or the first recombinant E. faecalis polypeptide antigen is an Ace polypeptide antigen.

Embodiment I-6. The immunogenic composition of any one of embodiments I-1 to I-4, wherein the at least one recombinant E. faecalis polypeptide antigen or the first recombinant E. faecalis polypeptide antigen is an AdcA polypeptide antigen.

Embodiment I-7. The immunogenic composition of any one of embodiments I-1 to I-4, wherein the at least one recombinant E. faecalis polypeptide antigen or the first recombinant E. faecalis polypeptide antigen is an AdcAII polypeptide antigen.

Embodiment I-8. The immunogenic composition of any one of embodiments I-1 to I-4, wherein the at least one recombinant E. faecalis polypeptide antigen or the first recombinant E. faecalis polypeptide antigen is an Ebp polypeptide antigen.

Embodiment I-9. The immunogenic composition of any one of embodiments I-1 to I-4, wherein the at least one recombinant E. faecalis polypeptide antigen or the first recombinant E. faecalis polypeptide antigen is an EfaA polypeptide antigen.

Embodiment I-15. The immunogenic composition of any one of embodiments I-1 to I-4, wherein the at least one recombinant E. faecalis polypeptide antigen or the first recombinant E. faecalis polypeptide antigen is an Esp polypeptide antigen.

Embodiment I-11. The immunogenic composition of any one of embodiments I-3 to I-15, wherein the at least one recombinant E. faecalis polypeptide antigen comprises a second recombinant E. faecalis polypeptide antigen, wherein the second recombinant E. faecalis polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-12. The immunogenic composition of any one of embodiments I-3 to I-11, wherein the at least one recombinant E. faecalis polypeptide antigen comprises a third recombinant E. faecalis polypeptide antigen, wherein the third recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-13. The immunogenic composition of any one of embodiments I-3 to I-12, wherein the at least one recombinant E. faecalis polypeptide antigen comprises a fourth E. faecalis polypeptide antigen, wherein the fourth E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-14. The immunogenic composition of any one of embodiments I-3 to I-13, wherein the at least one recombinant E. faecalis polypeptide antigen comprises a fifth recombinant E. faecalis polypeptide antigen, wherein the fifth recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-15. The immunogenic composition of any one of embodiments I-3 to I-14, wherein the at least one recombinant E. faecalis polypeptide antigen comprises a sixth recombinant E. faecalis polypeptide antigen, wherein the sixth recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment I-16. The immunogenic composition of any one of embodiments I-2 to I-15, wherein:
the Ace polypeptide antigen comprises or consists of the amino acid sequence of SEQ ID NO: 1 or SEQ ID NO: 13;
the AdcA polypeptide antigen comprises or consists of the amino acid sequence of SEQ ID NO: 2 or SEQ ID NO: 14;
the AdcAII polypeptide antigen comprises or consists of the amino acid sequence of SEQ ID NO: 3 or SEQ ID NO: 15;
the EbpA polypeptide antigen comprises or consists of the amino acid sequence of SEQ ID NO: 4 or SEQ ID NO: 16;
the EfaA polypeptide antigen comprises or consists of the amino acid sequence of SEQ ID NO: 5 or SEQ ID NO: 17; and
the Esp polypeptide antigen comprises or consists of the amino acid sequence of SEQ ID NO: 6 or SEQ ID NO: 18.

Embodiment I-17. The immunogenic composition of any one of embodiments I-1 to I-16, wherein the at least one recombinant E. faecalis polypeptide antigen comprises or consists of between 3 and 5 recombinant E. faecalis polypeptide antigens.

Embodiment I-18. The immunogenic composition of any one of embodiments I-3 to I-17, wherein the first recombinant E. faecalis polypeptide antigen is an AdcAII polypeptide antigen and the second recombinant E. faecalis polypeptide antigen is an EfaA polypeptide antigen.

Embodiment I-19. The immunogenic composition of any one of embodiments I-13 to I-17, wherein the first recombinant E. faecalis polypeptide antigen is an AdcAII polypeptide antigen, the second recombinant E. faecalis polypeptide antigen is an Esp polypeptide antigen, and the third recombinant E. faecalis polypeptide antigen is an Ace polypeptide antigen.

Embodiment I-20. The immunogenic composition of any one of embodiments I-13 to I-17, wherein the first recombinant E. faecalis polypeptide antigen is an AdcAII polypeptide antigen, the second recombinant E. faecalis polypeptide antigen is an EfaA polypeptide antigen, and the third recombinant E. faecalis polypeptide antigen is an EbpA polypeptide antigen.

Embodiment I-21. The immunogenic composition of any one of embodiments I-13 to I-17, wherein the first recombinant E. faecalis polypeptide antigen is an AdcAII polypeptide antigen, the second recombinant E. faecalis polypeptide antigen is an Ace polypeptide antigen, and the third recombinant E. faecalis polypeptide antigen is an EbpA polypeptide antigen.

Embodiment I-22. The immunogenic composition of any one of embodiments I-13 to I-17, wherein the first recombinant E. faecalis polypeptide antigen is an AdcA polypeptide antigen, the second recombinant E. faecalis polypeptide antigen is an EfaA polypeptide antigen, and the third recombinant E. faecalis polypeptide antigen is an EbpA polypeptide antigen.

Embodiment I-23. The immunogenic composition of any one of embodiments I-15 to I-17, wherein the first recombinant E. faecalis polypeptide antigen is an AdcA polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen, and the fifth recombinant E. faecalis polypeptide antigen is an Ace polypeptide antigen.

Embodiment I-24. The immunogenic composition of any one of embodiments I-14 to I-17, wherein the first recombinant E. faecalis polypeptide antigen is an AdcAII polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen; and the fifth recombinant E. faecalis polypeptide antigen is an Ace polypeptide antigen.

Embodiment I-25. The immunogenic composition of any one of embodiments I-1, I-2, I-4 to I-15, or I-16, wherein the composition does not comprise a second recombinant E. faecalis polypeptide antigen.

Embodiment I-26. The immunogenic composition of any one of embodiments I-3 to I-11 or I-16, wherein the composition does not comprise a third recombinant E. faecalis polypeptide antigen.

Embodiment I-27. The immunogenic composition of any one of embodiments I-3 to I-12 or I-16 to I-22, wherein the composition does not comprise a fourth recombinant E. faecalis polypeptide antigen.

Embodiment I-28. The immunogenic composition of any one of embodiments I-3 to I-13 or I-16 to I-22, wherein the composition does not comprise a fifth recombinant E. faecalis polypeptide antigen.

Embodiment I-29. The immunogenic composition of any one of embodiments I-3 to I-14 or I-16 to I-24, wherein the composition does not comprise a sixth recombinant E. faecalis polypeptide antigen.

Embodiment I-30. The immunogenic composition of any one of embodiments I-2 to I-24 or I-26 to I-29, wherein the AdcAII polypeptide antigen comprises an amino acid sequence that is at least 85%, at least 90%, or at least 95% identical to SEQ ID NO: 3 or SEQ ID NO: 15; and the EfaA polypeptide antigen comprises an amino acid sequence that is at least 95% identical to SEQ ID NO: 5 or SEQ ID NO: 17.

Embodiment I-31. The immunogenic composition of any one of embodiments I-2 to I-24 or I-26 to I-29, wherein the AdcAII polypeptide antigen comprises or consists of the amino acid sequence of SEQ ID NO: 3 or SEQ ID NO: 15; and the EfaA polypeptide antigen comprises or consists of the amino acid sequence of SEQ ID NO: 5 or SEQ ID NO: 17.

Embodiment I-32. The immunogenic composition of any one of embodiments I-2 to I-24 or I-27 to I-29, wherein the AdcAII polypeptide antigen comprises an amino acid sequence that is at least 85%, at least 90%, or at least 95% identical to SEQ ID NO: 3 or SEQ ID NO: 15; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

Embodiment I-33. The immunogenic composition of any one of embodiments I-2 to I-24 or I-27 to I-29, wherein the AdcAII polypeptide antigen comprises or consists of the amino acid sequence of SEQ ID NO: 3 or SEQ ID NO: 15; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

Embodiment I-34. The immunogenic composition of any one of embodiments I-2 to I-24 or I-27 to I-29, wherein the AdcAII polypeptide antigen comprises an amino acid sequence that is at least 85%, at least 90%, or at least 95% identical to SEQ ID NO: 3 or SEQ ID NO: 15; the EfaA polypeptide antigen comprises an amino acid sequence that is at least 95% identical to SEQ ID NO: 5 or SEQ ID NO: 17; and the EbpA polypeptide antigen comprises an amino acid sequence that is at least 95% identical to SEQ ID NO: 4 or SEQ ID NO: 16.

Embodiment I-35. The immunogenic composition of any one of embodiments I-2 to I-24 or I-27 to I-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

Embodiment I-36. The immunogenic composition of any one of embodiments I-2 to I-24 or I-27 to I-29, wherein the AdcAII polypeptide antigen comprises an amino acid sequence that is at least 85%, at least 90%, or at least 95% identical to SEQ ID NO: 3 or SEQ ID NO: 15; the Ace polypeptide antigen comprises an amino acid sequence that is at least 95% identical to SEQ ID NO: 1 or SEQ ID NO: 13; and the EbpA polypeptide antigen comprises an amino acid sequence that is at least 95% identical to SEQ ID NO: 4 or SEQ ID NO: 16.

Embodiment I-37. The immunogenic composition of any one of embodiments I-2 to I-24 or I-27 to I-29, wherein the AdcAII polypeptide antigen comprises or consists of the amino acid sequence of SEQ ID NO: 3 or SEQ ID NO: 15; the Ace polypeptide antigen comprises or consists of the amino acid sequence of SEQ ID NO: 1 or SEQ ID NO: 13; and the EbpA polypeptide antigen comprises or consists of the amino acid sequence of SEQ ID NO: 4 or SEQ ID NO: 16.

Embodiment I-38. The immunogenic composition of any one of embodiments I-2 to I-24 or I-27 to I-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

Embodiment I-39. The immunogenic composition of any one of embodiments I-2 to I-24 or I-27 to I-29, wherein the AdcA polypeptide antigen comprises an amino acid sequence that is at least 85%, at least 90%, or at least 95% identical to SEQ ID NO: 2 or SEQ ID NO: 14; the EfaA polypeptide antigen comprises an amino acid sequence that is at least 95% identical to SEQ ID NO: 5 or SEQ ID NO: 17; and the EbpA polypeptide antigen comprises an amino acid sequence that is at least 95% identical to SEQ ID NO: 4 or SEQ ID NO: 16.

Embodiment I-40. The immunogenic composition of any one of embodiments I-2 to I-24 or I-27 to I-29, wherein the AdcA polypeptide antigen comprises or consists of the amino acid sequence of SEQ ID NO: 2 or SEQ ID NO: 14; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

Embodiment I-41. The immunogenic composition of any one of embodiments I-2 to I-24 or I-28 to I-29, wherein the AdcA polypeptide antigen comprises an amino acid sequence that is at least 85%, at least 90%, or at least 95% identical to SEQ ID NO: 2 or SEQ ID NO: 14; the EfaA polypeptide antigen comprises an amino acid sequence that is at least 95% identical to SEQ ID NO: 5 or SEQ ID NO: 17; the EbpA polypeptide antigen comprises an amino acid sequence that is at least 95% identical to SEQ ID NO: 4 or SEQ ID NO: 16; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

Embodiment I-42. The immunogenic composition of any one of embodiments I-2 to I-24 or I-28 to I-29, wherein the AdcA polypeptide antigen comprises or consists of the amino acid sequence of SEQ ID NO: 2 or SEQ ID NO: 14; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

Embodiment I-43. The immunogenic composition of any one of embodiments I-2 to I-24 or I-28 to I-29, wherein the AdcAII polypeptide antigen comprises an amino acid sequence that is at least 85%, at least 90%, or at least 95% identical to SEQ ID NO: 3 or SEQ ID NO: 15; the EfaA polypeptide antigen comprises an amino acid sequence that is at least 95% identical to SEQ ID NO: 5 or SEQ ID NO: 17; the EbpA polypeptide antigen comprises an amino acid sequence that is at least 95% identical to SEQ ID NO: 4 or SEQ ID NO: 16; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

Embodiment I-44. The immunogenic composition of any one of embodiments I-2 to I-24 or I-28 to I-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

Embodiment I-45. The immunogenic composition of any one of embodiments I-1 to I-44, further comprising one or more adjuvants selected from alum, saponin, monophosphoryl lipid A (MPLA), TLR-agonist, or combinations thereof.

Embodiment I-46. The immunogenic composition of any one of embodiments I-1 to I-45, further comprising at least one excipient.

Embodiment I-47. The immunogenic composition of embodiment I-46, wherein the at least one excipient is selected from vehicles, solubilizers, emulsifiers, stabilizers, preservatives, isotonicity agents, buffer systems, dispersants, diluents, viscosity modifiers, and absorption enhancers.

Embodiment I-48. The immunogenic composition of any one of embodiments I-45 to I-47, wherein the composition is formulated as a sterile injectable solution.

Embodiment I-49. A method of inducing a protective immune response against an Enterococcus bacterium in a subject comprising administering the immunogenic composition of any one of embodiments I-1 to I-48 to the subject.

Embodiment I-50. The method of embodiment I-49, wherein the immunogenic composition induces an antibody response in the subject against the Enterococcus bacterium and does not induce an antibody response in the subject against human tissue.

Embodiment I-51. The immunogenic composition of any one of embodiments I-1 to I-48 for use in treating a disorder in a subject in need thereof.

Embodiment I-52. The immunogenic composition of any one of embodiments I-1 to I-48 for use in treating a disorder in a subject in need thereof, wherein the disorder is related to infection by an Enterococcus bacterium.

Embodiment I-53. Use of the immunogenic composition of any one of embodiments I-1 to I-48 for inducing a protective immune response against an Enterococcus bacterium in a subject.

Embodiment I-54. Use of the immunogenic composition of any one of embodiments I-1 to I-44 in the manufacture of a medicament for inducing a protective immune response against an Enterococcus bacterium in a subject.

Embodiment I-55. The method of embodiment I-49 or I-50, the immunogenic composition for use of embodiment I-51 or I-52, or the use of embodiment I-53 or I-54, wherein the Enterococcus bacterium is E. faecalis, E. faecium, or E. durans.

Embodiment I-56. The method of any one of embodiment I-49 or I-50, the immunogenic composition for use of embodiment I-51 or I-52, or the use of embodiment I-53 or I-54, wherein the Enterococcus bacterium is E. faecalis.

Embodiment I-57. The method of embodiments I-49 to I-50, the immunogenic composition for use of embodiment I-51 or I-52, or the use of embodiment I-53 or I-54, wherein the subject is at risk for, or is experiencing, root canal failure, endocarditis, bacteremia, urinary tract infections, prostatitis, intraabdominal infection, cellulitis, dysbiotic gastrointestinal tract, prosthetic joint infection, or wound infections.

Embodiment I-58. A method of any one of embodiment I-49 or I-50, the immunogenic composition for use of embodiment I-51 or I-52, or the use of embodiment I-53 or I-54, wherein the risk of root canal failure, endocarditis, bacteremia, urinary tract infections, prostatitis, intraabdominal infection, cellulitis, dysbiotic gastrointestinal tract, prosthetic joint infection, or wound infections in a subject is reduced compared to before administration of the immunogenic composition.

Embodiment II-1. An immunogenic composition comprising at least one recombinant Enterococcus faecium (E. faecium) polypeptide antigen or a fragment thereof.

Embodiment II-2. The immunogenic composition of embodiment II-1, wherein the at least one recombinant E. faecium polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen.

Embodiment II-3. The immunogenic composition of embodiment II-1 or II-2, wherein the at least one recombinant E. faecium polypeptide antigen comprises a first recombinant E. faecium polypeptide antigen and a second recombinant E. faecium polypeptide antigen.

Embodiment II-4. The immunogenic composition of embodiment II-3, wherein the first recombinant E. faecium polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-5. The immunogenic composition of any one of embodiments II-1 to II-4, wherein the at least one recombinant E. faecium polypeptide antigen or the first recombinant E. faecium polypeptide antigen is an Ace polypeptide antigen.

Embodiment II-6. The immunogenic composition of any one of embodiments II-1 to II-4, wherein the at least one recombinant E. faecium polypeptide antigen or the first recombinant E. faecium polypeptide antigen is an AdcA polypeptide antigen.

Embodiment II-7. The immunogenic composition of any one of embodiments II-1 to II-4, wherein the at least one recombinant E. faecium polypeptide antigen or the first recombinant E. faecium polypeptide antigen is an AdcAII polypeptide antigen.

Embodiment II-8. The immunogenic composition of any one of embodiments II-1 to II-4, wherein the at least one recombinant E. faecium polypeptide antigen or the first recombinant E. faecium polypeptide antigen is an Ebp polypeptide antigen.

Embodiment II-9. The immunogenic composition of any one of embodiments II-1 to II-4, wherein the at least one recombinant E. faecium polypeptide antigen or the first recombinant E. faecium polypeptide antigen is an EfaA polypeptide antigen.

Embodiment II-15. The immunogenic composition of any one of embodiments II-1 to II-4, wherein the at least one recombinant E. faecium polypeptide antigen or the first recombinant E. faecium polypeptide antigen is an Esp polypeptide antigen.

Embodiment II-11. The immunogenic composition of any one of embodiments II-3 to II-15, wherein the at least one recombinant E. faecium polypeptide antigen comprises a second recombinant E. faecium polypeptide antigen, wherein the second recombinant E. faecium polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-12. The immunogenic composition of any one of embodiments II-3 to II-11, wherein the at least one recombinant E. faecium polypeptide antigen comprises a third recombinant E. faecium polypeptide antigen, wherein the third recombinant E. faecium polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-13. The immunogenic composition of any one of embodiments II-3 to II-12, wherein the at least one recombinant E. faecium polypeptide antigen comprises a fourth E. faecium polypeptide antigen, wherein the fourth E. faecium polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-14. The immunogenic composition of any one of embodiments II-3 to II-13, wherein the at least one recombinant E. faecium polypeptide antigen comprises a fifth recombinant E. faecium polypeptide antigen, wherein the fifth recombinant E. faecium polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-15. The immunogenic composition of any one of embodiments II-3 to II-14, wherein the at least one recombinant E. faecium polypeptide antigen comprises a sixth recombinant E. faecium polypeptide antigen, wherein the sixth recombinant E. faecium polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

Embodiment II-16. The immunogenic composition of any one of embodiments II-2 to II-15, wherein:
the Ace polypeptide antigen comprises or consists of the amino acid sequence of SEQ ID NO: 7;
the AdcA polypeptide antigen comprises or consists of the amino acid sequence of SEQ ID NO: 8;
the AdcAII polypeptide antigen comprises or consists of the amino acid sequence of SEQ ID NO: 9;
the EbpA polypeptide antigen comprises or consists of the amino acid sequence of SEQ ID NO: 10;
the EfaA polypeptide antigen comprises or consists of the amino acid sequence of SEQ ID NO: 11; and
the Esp polypeptide antigen comprises or consists of the amino acid sequence of SEQ ID NO: 12.

Embodiment II-17. The immunogenic composition of any one of embodiments II-1 to II-16, wherein the at least one recombinant E. faecium polypeptide antigen comprises or consists of between 3 and 5 recombinant E. faecium polypeptide antigens.

Embodiment II-18. The immunogenic composition of any one of embodiments II-3 to II-17, wherein the first recombinant E. faecium polypeptide antigen is an AdcAII polypeptide antigen and the second recombinant E. faecium polypeptide antigen is an EfaA polypeptide antigen.

Embodiment II-19. The immunogenic composition of any one of embodiments II-13 to II-17, wherein the first recombinant E. faecium polypeptide antigen is an AdcAII polypeptide antigen, the second recombinant E. faecium polypeptide antigen is an Esp polypeptide antigen, and the third recombinant E. faecium polypeptide antigen is an Ace polypeptide antigen.

Embodiment II-20. The immunogenic composition of any one of embodiments II-13 to II-17, wherein the first recombinant E. faecium polypeptide antigen is an AdcAII polypeptide antigen, the second recombinant E. faecium polypeptide antigen is an EfaA polypeptide antigen, and the third recombinant E. faecium polypeptide antigen is an EbpA polypeptide antigen.

Embodiment II-21. The immunogenic composition of any one of embodiments II-13 to II-17, wherein the first recombinant E. faecium polypeptide antigen is an AdcAII polypeptide antigen, the second recombinant E. faecium polypeptide antigen is an Ace polypeptide antigen, and the third recombinant E. faecium polypeptide antigen is an EbpA polypeptide antigen.

Embodiment II-22. The immunogenic composition of any one of embodiments II-13 to II-17, wherein the first recombinant E. faecium polypeptide antigen is an AdcA polypeptide antigen, the second recombinant E. faecium polypeptide antigen is an EfaA polypeptide antigen, and the third recombinant E. faecium polypeptide antigen is an EbpA polypeptide antigen.

Embodiment II-23. The immunogenic composition of any one of embodiments II-15 to II-17, wherein the first recombinant E. faecium polypeptide antigen is an AdcA polypeptide antigen, the second recombinant E. faecium polypeptide antigen is an EfaA polypeptide antigen, the third recombinant E. faecium polypeptide antigen is an EbpA polypeptide antigen, the fourth recombinant E. faecium polypeptide antigen is an Esp polypeptide antigen, and the fifth recombinant E. faecium polypeptide antigen is an Ace polypeptide antigen.

Embodiment II-24. The immunogenic composition of any one of embodiments II-14 to II-17, wherein the first recombinant E. faecium polypeptide antigen is an AdcAII polypeptide antigen, the second recombinant E. faecium polypeptide antigen is an EfaA polypeptide antigen, the third recombinant E. faecium polypeptide antigen is an EbpA polypeptide antigen, the fourth recombinant E. faecium polypeptide antigen is an Esp polypeptide antigen; and the fifth recombinant E. faecium polypeptide antigen is an Ace polypeptide antigen.

Embodiment II-25. The immunogenic composition of any one of embodiments II-1, II-2, II-4 to II-15, or II-16, wherein the composition does not comprise a second recombinant E. faecium polypeptide antigen.

Embodiment II-26. The immunogenic composition of any one of embodiments II-3 to II-11 or II-16, wherein the composition does not comprise a third recombinant E. faecium polypeptide antigen.

Embodiment II-27. The immunogenic composition of any one of embodiments II-3 to II-12 or II-16 to II-22, wherein the composition does not comprise a fourth recombinant E. faecium polypeptide antigen.

Embodiment II-28. The immunogenic composition of any one of embodiments II-3 to II-13 or II-16 to II-22, wherein the composition does not comprise a fifth recombinant E. faecium polypeptide antigen.

Embodiment II-29. The immunogenic composition of any one of embodiments II-3 to II-14 or II-16 to II-24, wherein the composition does not comprise a sixth recombinant E. faecium polypeptide antigen.

Embodiment II-30. The immunogenic composition of any one of embodiments II-2 to II-24 or II-26 to II-29, wherein the AdcAII polypeptide antigen comprises an amino acid sequence that is at least 85%, at least 90%, or at least 95% identical to SEQ ID NO: 9; and the EfaA polypeptide antigen comprises an amino acid sequence that is at least 95% identical to SEQ ID NO: 11.

Embodiment II-31. The immunogenic composition of any one of embodiments II-2 to II-24 or II-26 to II-29, wherein the AdcAII polypeptide antigen comprises or consists of the amino acid sequence of SEQ ID NO: 3 or SEQ ID NO: 15; and the EfaA polypeptide antigen comprises or consists of the amino acid sequence of SEQ ID NO: 5 or SEQ ID NO: 17.

Embodiment II-32. The immunogenic composition of any one of embodiments II-2 to II-24 or II-27 to II-29, wherein the AdcAII polypeptide antigen comprises an amino acid sequence that is at least 85%, at least 90%, or at least 95% identical to SEQ ID NO: 9; the Esp polypeptide antigen comprises an amino acid sequence that is at least 95% identical to SEQ ID NO: 12; and the Ace polypeptide antigen comprises an amino acid sequence that is at least 95% identical to SEQ ID NO: 7.

Embodiment II-33. The immunogenic composition of any one of embodiments II-2 to II-24 or II-27 to II-29, wherein the AdcAII polypeptide antigen comprises or consists of the amino acid sequence of SEQ ID NO: 9; the Esp polypeptide antigen comprises or consists of the amino acid sequence of SEQ ID NO: 12; and the Ace polypeptide antigen comprises or consists of the amino acid sequence of SEQ ID NO: 7.

Embodiment II-34. The immunogenic composition of any one of embodiments II-2 to II-24 or II-27 to II-29, wherein the AdcAII polypeptide antigen comprises an amino acid sequence that is at least 85%, at least 90%, or at least 95% identical to SEQ ID NO: 9; the EfaA polypeptide antigen comprises an amino acid sequence that is at least 95% identical to SEQ ID NO: 11; and the EbpA polypeptide antigen comprises an amino acid sequence that is at least 95% identical to SEQ ID NO: 10.

Embodiment II-35. The immunogenic composition of any one of embodiments II-2 to II-24 or II-27 to II-29, wherein the AdcAII polypeptide antigen comprises or consists of the amino acid sequence of SEQ ID NO: 9; the EfaA polypeptide antigen comprises or consists of the amino acid sequence of SEQ ID NO: 11; and the EbpA polypeptide antigen comprises or consists of the amino acid sequence of SEQ ID NO: 10.

Embodiment II-36. The immunogenic composition of any one of embodiments II-2 to II-24 or II-27 to II-29, wherein the AdcAII polypeptide antigen comprises an amino acid sequence that is at least 85%, at least 90%, or at least 95% identical to SEQ ID NO: 9; the Ace polypeptide antigen comprises an amino acid sequence that is at least 95% identical to SEQ ID NO: 7; and the EbpA polypeptide antigen comprises an amino acid sequence that is at least 95% identical to SEQ ID NO: 10.

Embodiment II-37. The immunogenic composition of any one of embodiments II-2 to II-24 or II-27 to II-29, wherein the AdcAII polypeptide antigen comprises or consists of the amino acid sequence of SEQ ID NO: 9; the Ace polypeptide antigen comprises or consists of the amino acid sequence of SEQ ID NO: 7; and the EbpA polypeptide antigen comprises or consists of the amino acid sequence of SEQ ID NO: 10.

Embodiment II-38. The immunogenic composition of any one of embodiments II-2 to II-24 or II-27 to II-29, wherein the AdcAII polypeptide antigen comprises or consists of the amino acid sequence of SEQ ID NO: 9; the EfaA polypeptide antigen comprises or consists of the amino acid sequence of SEQ ID NO: 11; and the EbpA polypeptide antigen comprises or consists of the amino acid sequence of SEQ ID NO: 10.

Embodiment II-39. The immunogenic composition of any one of embodiments II-2 to II-24 or II-27 to II-29, wherein the AdcA polypeptide antigen comprises an amino acid sequence that is at least 85%, at least 90%, or at least 95% identical to SEQ ID NO: 8; the EfaA polypeptide antigen comprises an amino acid sequence that is at least 95% identical to SEQ ID NO: 11; and the EbpA polypeptide antigen comprises an amino acid sequence that is at least 95% identical to SEQ ID NO:10.

Embodiment II-40. The immunogenic composition of any one of embodiments II-2 to II-24 or II-27 to II-29, wherein the AdcA polypeptide antigen comprises or consists of the amino acid sequence of SEQ ID NO: 8; the EfaA polypeptide antigen comprises or consists of the amino acid sequence of SEQ ID NO: 11; and the EbpA polypeptide antigen comprises or consists of the amino acid sequence of SEQ ID NO: 10.

Embodiment II-41. The immunogenic composition of any one of embodiments II-2 to II-24 or II-28 to II-29, wherein the AdcA polypeptide antigen comprises an amino acid sequence that is at least 85%, at least 90%, or at least 95% identical to SEQ ID NO: 8; the EfaA polypeptide antigen comprises an amino acid sequence that is at least 95% identical to SEQ ID NO: 11; the EbpA polypeptide antigen comprises an amino acid sequence that is at least 95% identical to SEQ ID NO: 10; the Esp polypeptide antigen comprises an amino acid sequence that is at least 95% identical to SEQ ID NO: 12; and the Ace polypeptide antigen comprises an amino acid sequence that is at least 95% identical to SEQ ID NO: 7.

Embodiment II-42. The immunogenic composition of any one of embodiments II-2 to II-24 or II-28 to II-29, wherein the AdcA polypeptide antigen comprises or consists of the amino acid sequence of SEQ ID NO: 8; the EfaA polypeptide antigen comprises or consists of the amino acid sequence of SEQ ID NO: 11; the EbpA polypeptide antigen comprises or consists of the amino acid sequence of SEQ ID NO: 10; the Esp polypeptide antigen comprises or consists of the amino acid sequence of SEQ ID NO: 12; and the Ace polypeptide antigen comprises or consists of the amino acid sequence of SEQ ID NO: 7.

Embodiment II-43. The immunogenic composition of any one of embodiments II-2 to II-24 or II-28 to II-29, wherein the AdcAII polypeptide antigen comprises an amino acid sequence that is at least 85%, at least 90%, or at least 95% identical to SEQ ID NO: 9; the EfaA polypeptide antigen comprises an amino acid sequence that is at least 95% identical to SEQ ID NO: 11; the EbpA polypeptide antigen comprises an amino acid sequence that is at least 95% identical to SEQ ID NO: 10; the Esp polypeptide antigen comprises an amino acid sequence that is at least 95% identical to SEQ ID NO: 12; and the Ace polypeptide antigen comprises an amino acid sequence that is at least 95% identical to SEQ ID NO: 7.

Embodiment II-44. The immunogenic composition of any one of embodiments II-2 to II-24 or II-28 to II-29, wherein the AdcAII polypeptide antigen comprises or consists of the amino acid sequence of SEQ ID NO: 9; the EfaA polypeptide antigen comprises or consists of the amino acid sequence of SEQ ID NO: 11; the EbpA polypeptide antigen comprises or consists of the amino acid sequence of SEQ ID NO: 10; the Esp polypeptide antigen comprises or consists of the amino acid sequence of SEQ ID NO: 12; and the Ace polypeptide antigen comprises or consists of the amino acid sequence of SEQ ID NO: 7.

Embodiment II-45. The immunogenic composition of any one of embodiments II-1 to II-44, further comprising one or more adjuvants selected from alum, saponin, monophosphoryl lipid A (MPLA), TLR-agonist, or combinations thereof.

Embodiment II-46. The immunogenic composition of any one of embodiments II-1 to II-45, further comprising at least one excipient.

Embodiment II-47. The immunogenic composition of embodiment II-46, wherein the at least one excipient is selected from vehicles, solubilizers, emulsifiers, stabilizers, preservatives, isotonicity agents, buffer systems, dispersants, diluents, viscosity modifiers, and absorption enhancers.

Embodiment II-48. The immunogenic composition of any one of embodiments II-45 to II-47, wherein the composition is formulated as a sterile injectable solution.

Embodiment II-49. A method of inducing a protective immune response against an Enterococcus bacterium in a subject comprising administering the immunogenic composition of any one of embodiments II-1 to II-48 to the subject.

Embodiment II-50. The method of embodiment II-49, wherein the immunogenic composition induces an antibody response in the subject against the Enterococcus bacterium and does not induce an antibody response in the subject against human tissue.

Embodiment II-51. The immunogenic composition of any one of embodiments II-1 to II-48 for use in treating a disorder in a subject in need thereof.

Embodiment II-52. The immunogenic composition of any one of embodiments II-1 to II-48 for use in treating a disorder in a subject in need thereof, wherein the disorder is related to infection by an Enterococcus bacterium.

Embodiment II-53. Use of the immunogenic composition of any one of embodiments II-1 to II-48 for inducing a protective immune response against an Enterococcus bacterium in a subject.

Embodiment II-54. Use of the immunogenic composition of any one of embodiments II-1 to II-44 in the manufacture of a medicament for inducing a protective immune response against an Enterococcus bacterium in a subject.

Embodiment II-55. The method of embodiment II-49 or II-50, the immunogenic composition for use of embodiment II-51 or II-52, or the use of embodiment II-53 or II-54, wherein the Enterococcus bacterium is E. faecalis, E. faecium, or E. durans.

Embodiment II-56. The method of any one of embodiment II-49 or II-50, the immunogenic composition for use of embodiment II-51 or II-52, or the use of embodiment II-53 or II-54, wherein the Enterococcus bacterium is E. faecium.

Embodiment II-57. The method of embodiments II-49 to II-50, the immunogenic composition for use of embodiment II-51 or II-52, or the use of embodiment II-53 or II-54, wherein the subject is at risk for, or is experiencing, root canal failure, endocarditis, bacteremia, urinary tract infections, prostatitis, intraabdominal infection, cellulitis, dysbiotic gastrointestinal tract, prosthetic joint infection, or wound infections.

Embodiment II-58. A method of any one of embodiment II-49 or II-50, the immunogenic composition for use of embodiment II-51 or II-52, or the use of embodiment II-53 or II-54, wherein the risk of root canal failure, endocarditis, bacteremia, urinary tract infections, prostatitis, intraabdominal infection, cellulitis, dysbiotic gastrointestinal tract, prosthetic joint infection, or wound infections in a subject is reduced compared to before administration of the immunogenic composition.

Embodiment II-59. The immunogenic composition of any one of Embodiments I-1 to I-48 or II-1 to II-48, the method of any one of Embodiments I-49 to I-50, I-55 to I-58, II-49 to II-50, or II-55 to II-58, the immunogenic composition for use of Embodiments I-51, I-52, II-51, or II-52, or the use of Embodiments I-53, I-54, II-53, or II-54, wherein one or more of the at least one polypeptide antigen is a fragment of the polypeptide antigen.

Embodiment II-60. The immunogenic composition, method, immunogenic composition for use, or use of Embodiments I-59 or II-59, wherein the fragment of the at least one polypeptide antigen is at least 15%, 15%, 20%, 25%, 30%, 35%, 40%, 45%, 50%, 55%, 60%, 65%, 70%, 75%, 80%, 85%, 90%, or 95% of the length of the at least one polypeptide antigen.

Embodiment II-61. Invention of product, process, system, kit or use, characterized by one or more elements disclosed in the application.

### EXAMPLES

Unless defined otherwise, all technical and scientific terms used herein have the commonly understood meaning. Practitioners are particularly directed to Green & Sambrook (eds.) Molecular Cloning: A Laboratory Manual, 4th ed., Cold Spring Harbor Laboratory Press, Cold Spring Harbor, N.Y. (2012); Ausubel et al., Current Protocols in Molecular Biology (Supplement 99) (New York: John Wiley & Sons, 2012), and Plotkin et al., Vaccines, Sixth Ed. (London: Elsevier, 2013). Examples of appropriate molecular techniques for generating recombinant nucleic acids, cloning, activating and derivatizing biomolecules, purifying and identifying proteins and peptides, and other pertinent techniques are also described and/or cited in U.S. Patent Publication No. US 2018/0333484 A1 to Fairman et al. (Vaxcyte, Inc.), and PCT Publication No. WO 2014/172631 A2, each of which is incorporated by reference herein. For examples of techniques and components necessary for parenteral administration of biomolecules described herein, practitioners are directed to Remington, Essentials of Pharmaceutics, Pharmaceutical Press, London (2012). Methods for cell-free protein synthesis are also described in Spirin & Swartz (2008) Cell-free Protein Synthesis, Wiley-VCH, Weinheim, Germany.

### Example 1: Enterococcus Antigen Expression Screen

To determine how different Enterococcus polypeptide antigens express in a cell-free platform, microscale cell-free protein synthesis reactions are performed in the presence of radiolabeled leucine, the incorporation of which allows the assessment of total protein and soluble protein expression in the reaction mixture.

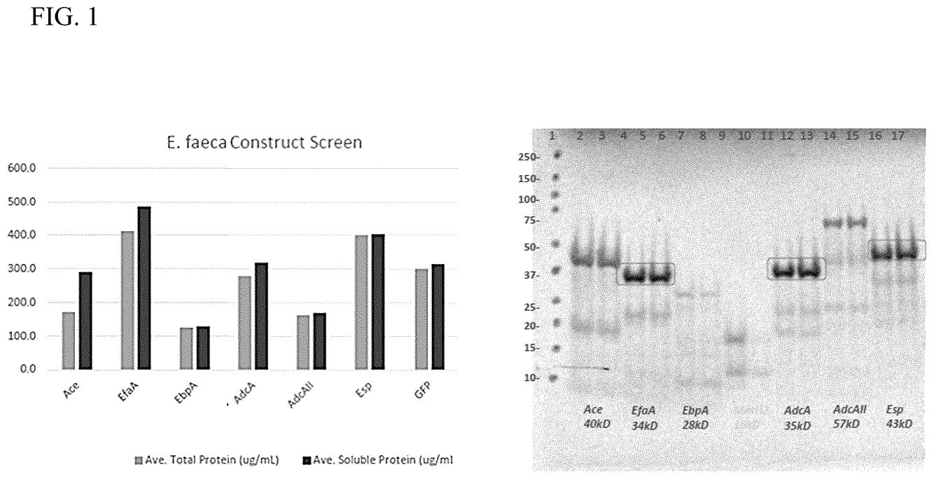

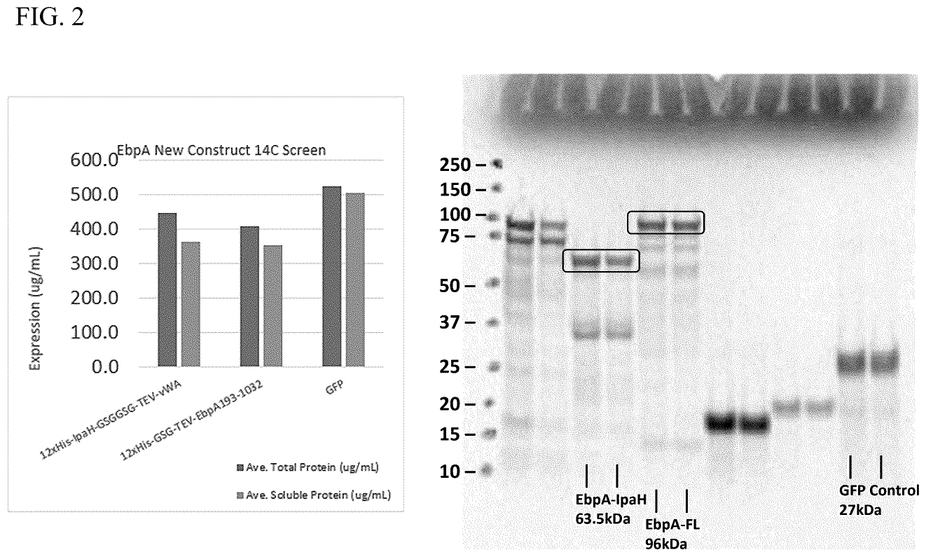

Microscale expression test reactions were performed in 150 μL of supermix with 2% ¹⁴C-labeled leucine, plasmid DNA for each E. faecalis polypeptide construct, cell-free E. coli extract, T7 polymerase, and water. Test reactions were performed with constructs corresponding to SEQ ID NOs: 13-18. FIG. 1 shows the total average protein and average soluble protein of the expressed polypeptides (μg/mL) as well as the accompanying SDS page gels. Most E. faecalis polypeptide antigens expressed with a high amount of soluble protein. Because the initial EbpA construct (vWA domain, 28 kD) did not express well, additional constructs were expressed (FIG. 2). The EbpA construct (aa193-1532) provided better expression than the vWA domain fragment.

### Example 2: Enterococcus Antigen Expression and Purification Scaleup

Cell-free extracts were thawed to room temperature and incubated with 50 μM iodoacetamide for 30 min, as previously described (Knapp et al. 2007). OCFS reactions were run at 25° C. for up to 15 h containing 30% (v/v) iodoacetamide-treated extract with 8 mM magnesium glutamate, 15 mM ammonium glutamate, 130 mM potassium glutamate, 35 mM sodium pyruvate, 1.2 mM AMP, 0.86 mM each of GMP, UMP, and CMP, 2 mM amino acids (1 mM for tyrosine), 4 mM sodium oxalate, 1 mM putrescine, 1.5 mM spermidine, 15 mM potassium phosphate, 150 nM T7 RNAP, 2-15 μg/mL plasmid DNA template, 1-15 μM E. coli DsbC. Reduced (GSH) and oxidized (GSSG) glutathione were added to a total concentration of 5 mM. The initial redox potential was calculated using the Nernst equation with E0=205 mV as the standard potential of the GSH/GSSG couple at 30° C. and pH 7.

Proteins produced at 250 μL scale or less were monitored by incorporation of 1-[U-14C]-leucine by measuring the TCA-precipitable soluble and total protein in 24-well plates or Petri dishes without shaking (Voloshin and Swartz, 2005), or in 96-well plates at 30 μL scale (with shaking). Proteins were analyzed by reducing or non-reducing 12% SDS-PAGE gels with Sypro staining according to the manufacturer s recommendations and analyzed by autoradiography using a PhosphoImager.

Proteins produced at the larger scale were plated evenly in 20 cm petri dishes, forming a thin layer at the bottom, and the plates were incubated at 25° C. for 14-16 hours. The pool was harvested the next day at 7,500×g for 35 min, the supernatant was collected, and the salt content was adjusted to 50 mM Tris pH8, 150 mM NaCl, and15 mM imidazole, prior to being subjected to a 0.22 μM filtration

To purify the polypeptide antigens, NiCapture was used to bind the His tagged protein. 5 mL HisTrap excel columns were equilibrated in Buffer A (50 mM Tris pH8, 150 mM NaCl, and 15 mM imidazole) and washed to baseline with 15 CV of Buffer A, 15CV Buffer A2 (+0.05% Triton X-150 for endotoxin removal), followed by 12 CV of Buffer A to remove the detergent. The polypeptide antigens were eluted using a 150% Buffer B gradient (50 mM Tris pH8, 150 mM NaCl, and 300 mM imidazole). The fractions corresponding to the elution peak were pooled, and the TeV sequences were cleaved using 1:15 TEV protease to protein. Dialysis was performed overnight in 1× TBS. The sample was retrieved and passed over 1 mL HisTrap using 1× TBS as Buffer A, and 300 mM imidazole as B1. The flowthrough was collected and concentrated using a 15 kD cut-off Amicon. SDS-page gels were run with flow-through, washed and eluted from the first Akta purification, and again after Tev cleavage, also stained with Coomassie Blue for size and purity as well as TeV Cleavage confirmation. The Endotoxin level was tested using Charles River Endosafe Nexgen-PTS150 Endotoxin testing detection system. Aliquots of purified polypeptide antigen were prepared, labeled, and stored at −80 C.

Table 2 shows the results of select E. faecalis polypeptide antigen 150 mL-scale expression/purification and TEV cleavage.

TABLE 2
Exemplary Enterococcus Polypeptide Antigens

| | | | TEV | | Aliquots | Aliquots | | | Total | |
|---|---|---|---|---|---|---|---|---|---|---|
| SEQ | Antigen | MW | Treated | Concentration | # of | Volume | Endotoxin | | Yield | Storage |
| ID NO: | name | kD | (Y/N) | (mg/mL) | Tubes | (mL) | (EU/ug) | Buffer | mg | temp |
| 13 | Ace | 39.81 | Y | 4.2 | 17 | 0.25 | 0.11 | 1 × TBS | 17.9 | −80° C. |
| 14 | AdcA | 34.48 | Y | 2.7 | 35 | 0.25 | 0.27 | 1 × TBS | 23.8 | −80° C. |
| 15 | AdcAII | 57.03 | Y | 2.0 | 44 | 0.25 | 0.27 | 1 × TBS | 22.2 | −80° C. |
| 16 | EbpA | 96.00 | Y | 3.5 | 30 | 0.25 | 0.19 | 1 × TBS | 26.4 | −80° C. |
| 17 | EfaA | 34.33 | Y | 15.25 | 16 | 0.15 | 0.06 | 1 × TBS | 36.6 | −80° C. |
| 18 | Esp | 43.28 | Y | 3.88 | 22 | 0.20 | 0.22 | 1 × TBS + | 17.1 | −80° C. |
| | | | | | | | | 15% Gly | | |

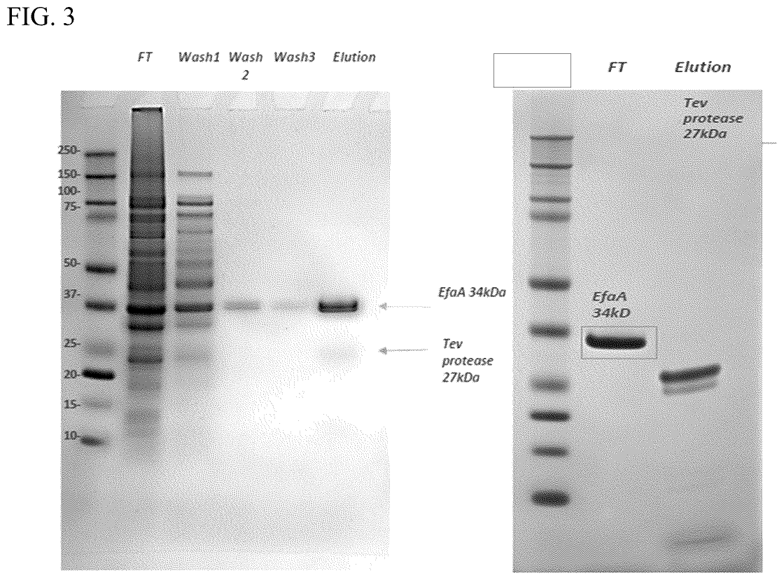

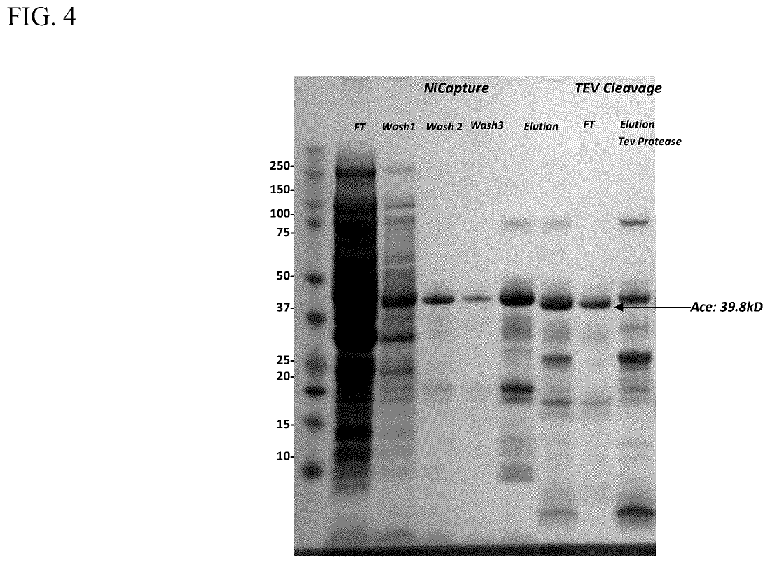

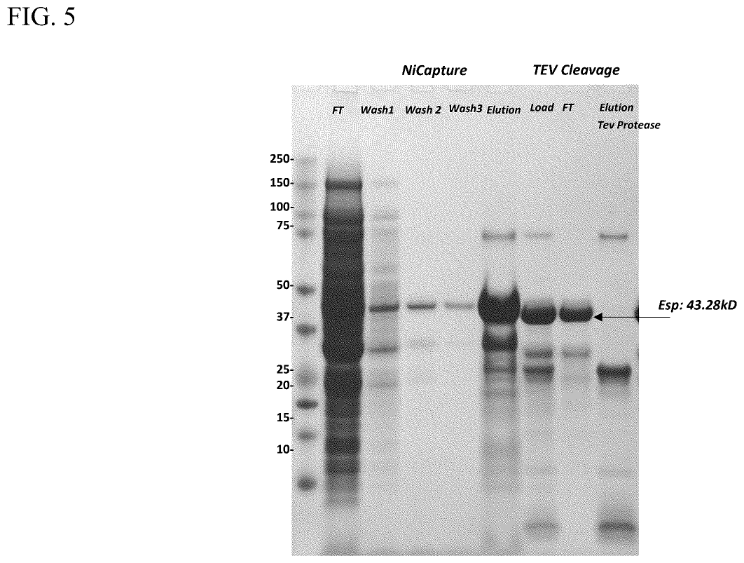

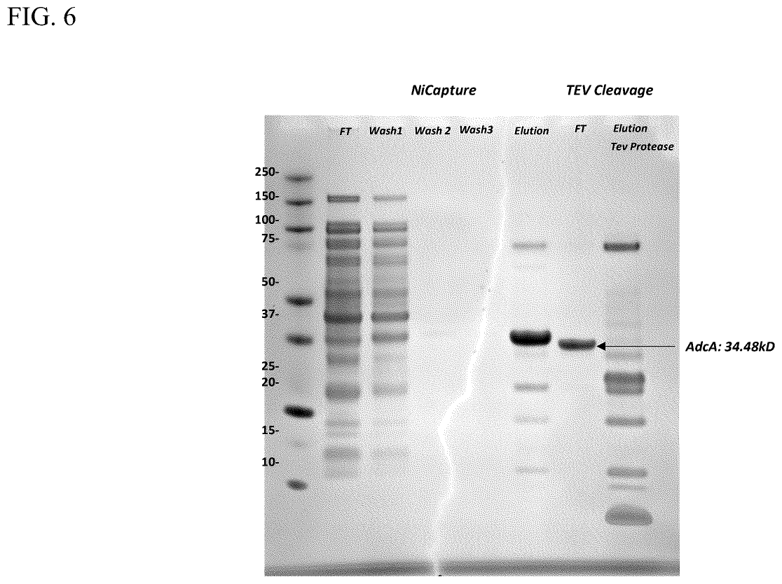

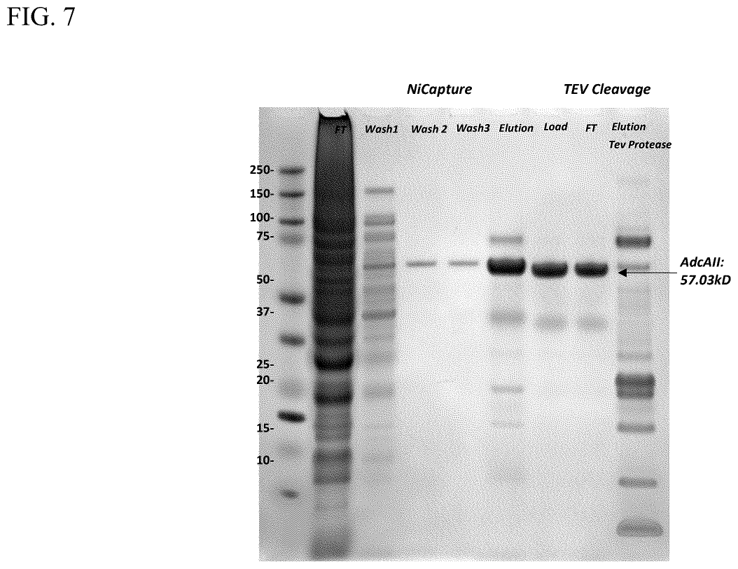

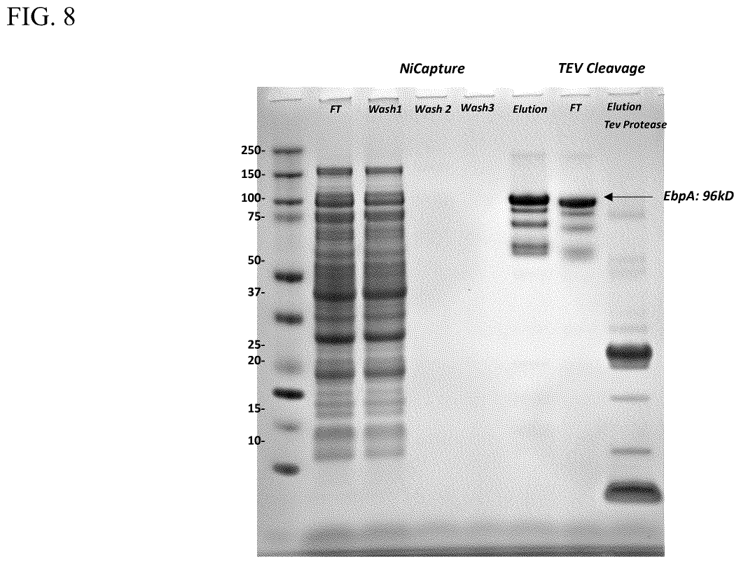

FIGS. 3-8 show gels after expression/purification and TEV cleavage, respectively, for exemplary E. faecalis polypeptide antigens. It was found that the Esp polypeptide antigen of SEQ ID NO: 18 was unstable during multiple freeze/thaw cycles. Adjusting the storage buffer to 1×TBS with 15% glycerol increased the stability of the polypeptide.

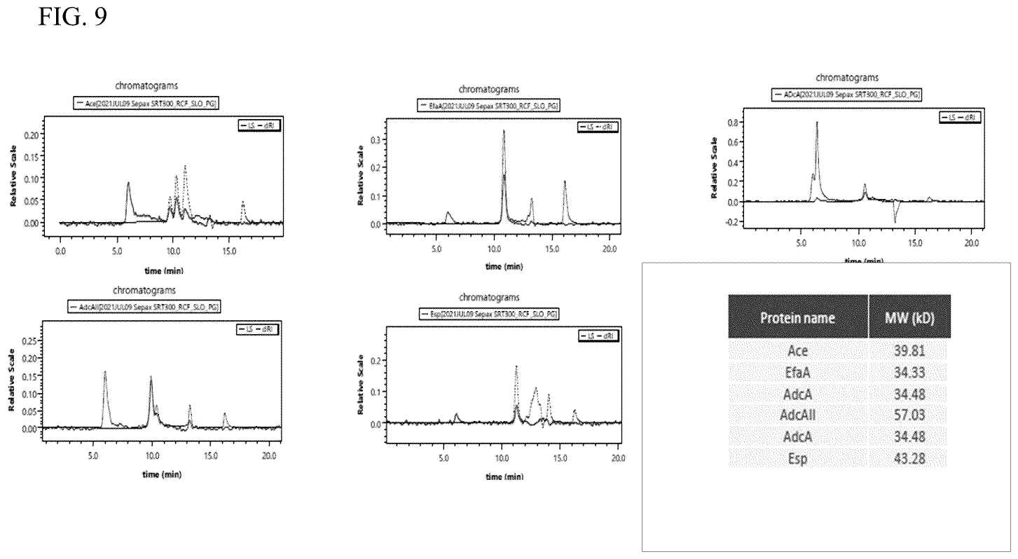

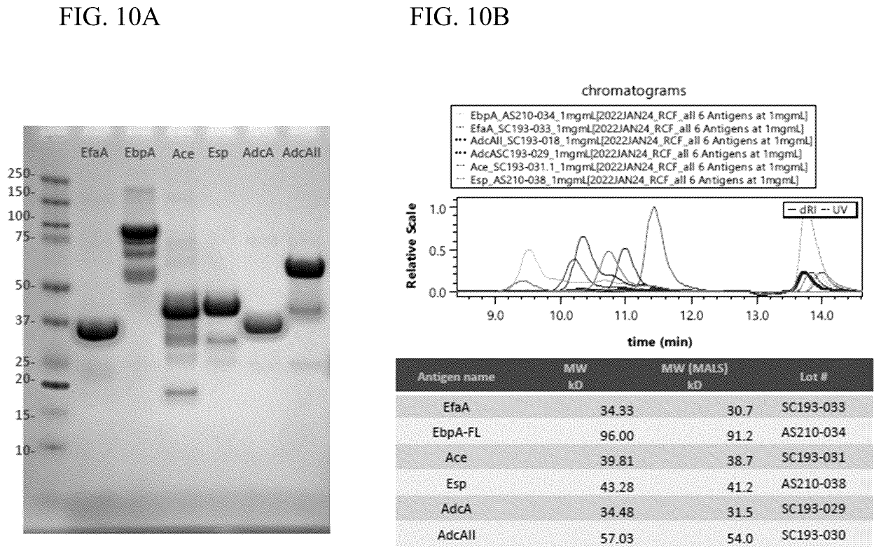

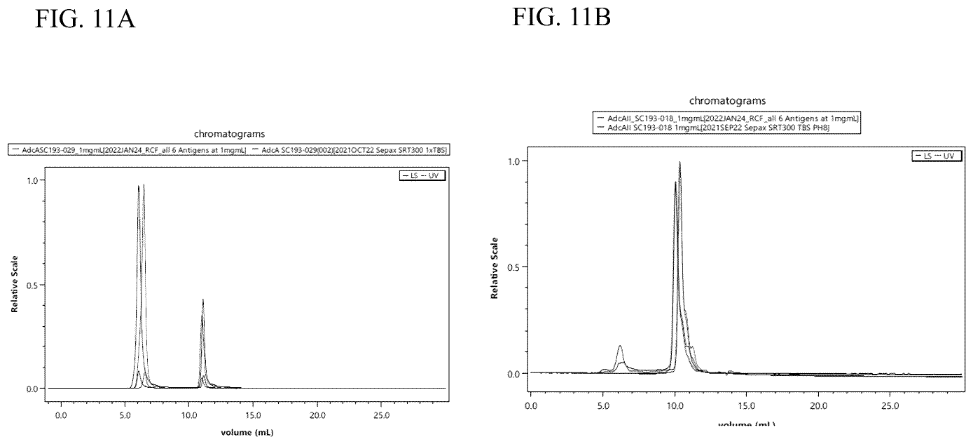

Multi-angle light scattering coupled with size exclusion chromatography (SEC-MALS) was used to confirm the molecular weight of the isolated exemplary polypeptide antigens. FIG. 9 shows the chromatographs for the SEC-MALS molecular weight analysis for polypeptide antigens of SEQ ID NOs: 12-18.

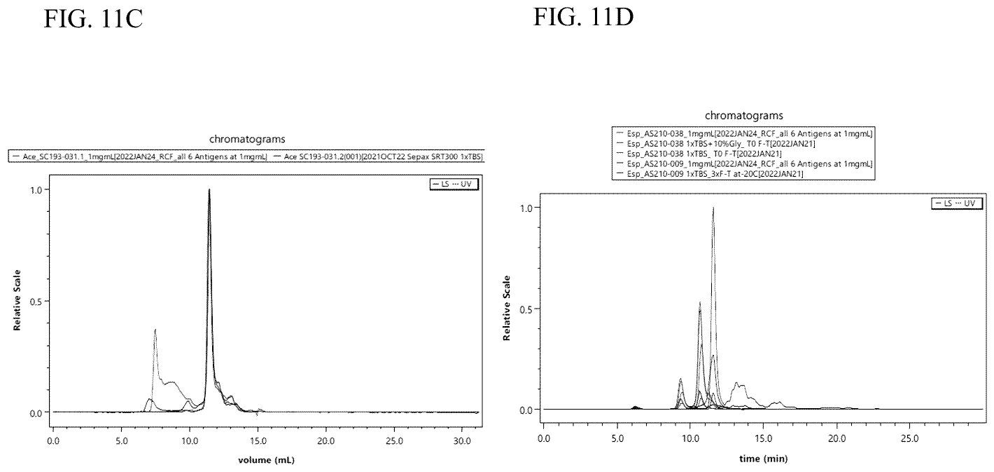

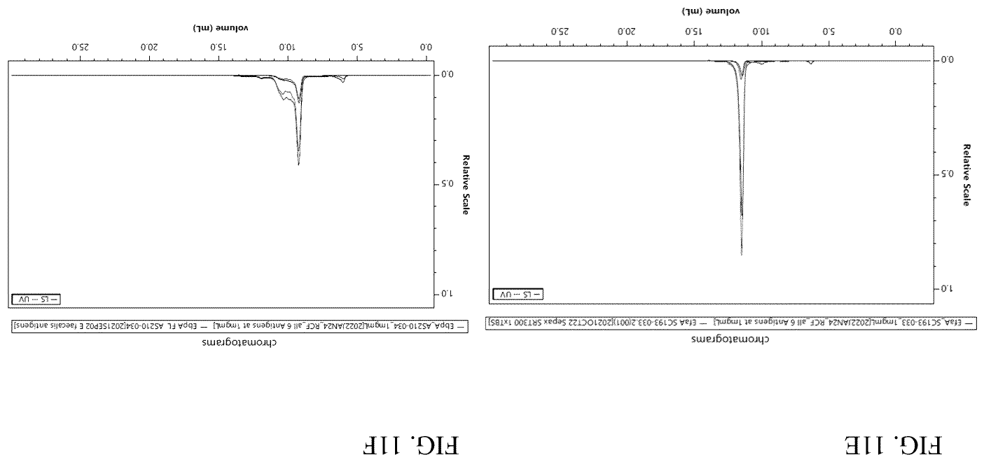

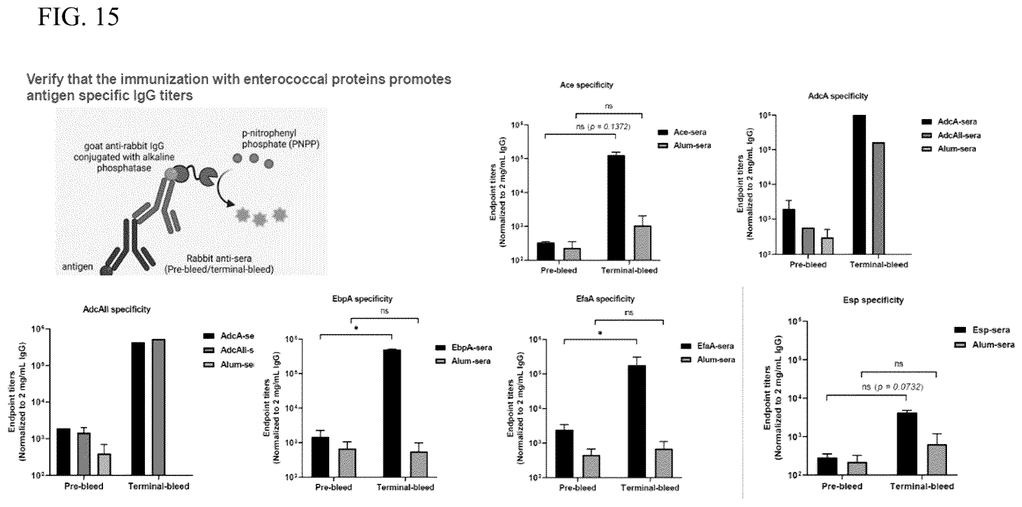

Final confirmation of molecular weight was obtained by SEC-MALS for the polypeptide antigens used in animal studies. FIGS. 15A and 15B show SDS-PAGE and SEC-MALS data, respectively, for this batch of polypeptide antigen expression. Samples of each polypeptide antigen were also subjected to stability studies comprising 3 freeze-thaw cycles. FIGS. 11A-11F show the SEC-MALS data after conducting the freeze thaw cycles on samples of E. faecalis AdcA, AdcAII, Ace, Esp, EfaA, and EbpA, respectively. FIG. 11A shows that AdcA is relatively stable, with only a small decrease in the main peak magnitude and molecular weight. FIG. 11B shows that AdcAII is also relatively stable, with only a small decrease in the main peak magnitude and a slight increase in molecular weight. Ace showed consistent SEC-MALS peaks (FIG. 11C). FIG. 11D shows the results of stability studies with two different Esp samples. As described earlier, and now shown in FIG. 11D, Esp was not stable until 15% glycerol was added to the storage buffer. FIGS. 11E and 11F show that EfaA and EpbA, respectively, are relatively stable to freeze thaw cycles.

### Example 3: Immunization Study in CD-1 Mice with Individual Exemplary E. faecalis Immunogenic Polypeptide Antigens

Seven groups of female CD-1 mice, each comprising 15 mice, were dosed with 5 μg of each antigen (EfaA, Ace, Esp, AdcA, AdcAII, and alum control) on each of Days 0, 7, and 14. Terminal bleed was conducted on Day 21, and the serum was analyzed.

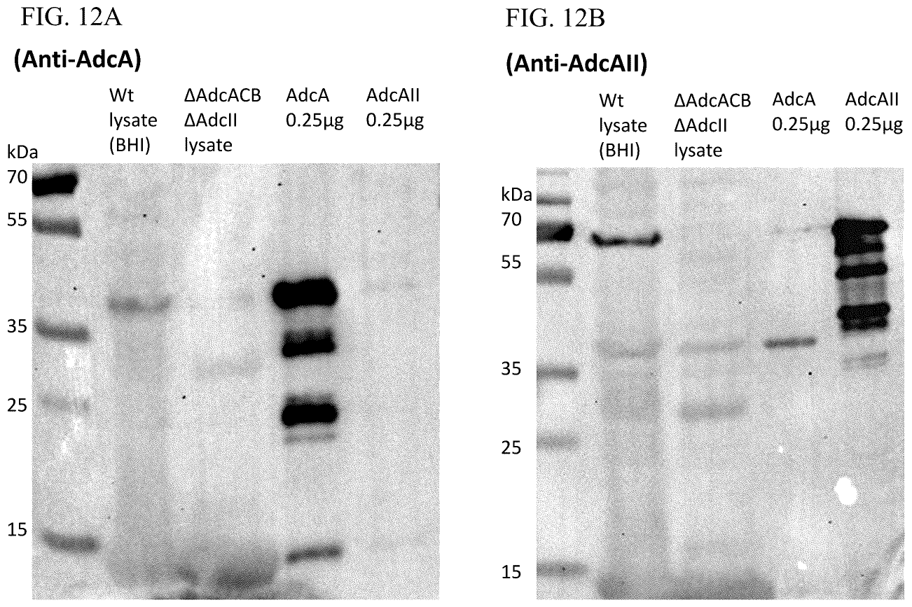

Immunoblots are developed to analyze antigen specificity. Polypeptides are loaded and run on an SDS-gel and transferred to a PVDF membrane. The membrane is then probed with the collected mouse serum containing anti-sera (primary antibodies against each antigen) which then binds to proteins on the PVDF membrane if they are present. A goat anti-mouse sera conjugated with horseradish peroxidase (secondary antibody) allows for visualization of the protein band. FIG. 12A shows in Lane 1: “Wt lysate (BHI)” that the mouse antisera generated against the AdcA antigen detects the presence of AdcA protein in the cell lysate generated from wild-type E. faecalis OG1RF grown on Blood Heart Infusion broth, although the band elutes slightly higher for the expected Mw of 35 kDa for AdcA. Lane 2: “AAdcACB/AAdcA lysate” probes cell lysate generated from AAdcACB/AAdcA null mutant strain of E. faecalis OG1RF and shows the absence of AdcA protein, as is expected. The last two lanes are 0.25 μg of each of the antigens used for immunization of the mice. Lane 3: “AdcA 0.25 μg” shows that the AdcA antigen is detected by the antisera, and that there are some fragments present. Lane 4: “AdcAII 0.25 μg” shows that there is little to no cross-reactivity of the AdcA antisera with the AdcAII antigen. FIG. 12B shows in Lane 1: “Wt lysate (BHI)” that the mouse antisera generated against the AdcAII antigen detects the presence of AdcAII protein in the cell lysate generated from wild-type E. faecalis OG1RF grown on Blood Heart Infusion broth, although the band elutes slightly higher for the expected Mw of 57 kDa for AdcAII. Lane 2: “AAdcACB AAdcA lysate” probes cell lysate generated from AAdcACB/AAdcA null mutant strain of E. faecalis OG1RF and interestingly detects some presence of AdcA protein, which suggest cross-reactivity of AdcAII antisera to AdcA. The last two lanes are 0.25 μg of each of the antigens used for immunization of the mice. Lane 3: “AdcA 0.25 μg” shows that some of the AdcA antigen bands are detected by the AdcAII antisera, indicating cross-reactivity. Lane 4: “AdcAII 0.25 μg” shows that the AdcAII antigen is detected by the AdcAII antisera, as is to be expected. Overall, the Western immunoblots indicate that expression of antigens by E. faecalis OGRFI is conditional, that the antisera generated against the AdcA antigen is not cross-reactive to the AdcAII antigen, whereas, the antisera generated against the AdcAII antigen is cross-reactive to the AdcA antigen.

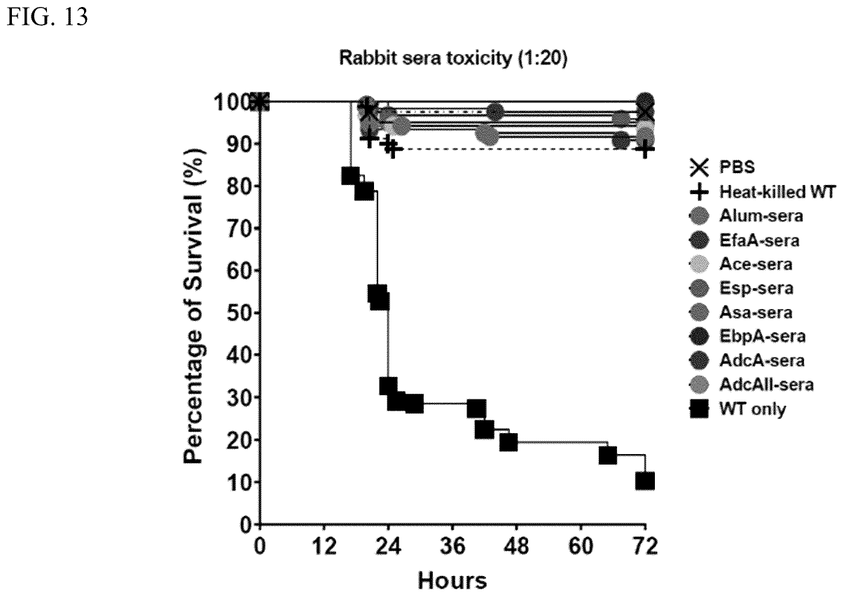

A G. mellonella immunization model was used to analyze the neutralizing and opsonizing capacities of the mouse anti-sera to modulate E. faecalis virulence. Details on this model may be found, for example, in Lam, et al. Bacterial Virulence, Methods in Mol. Bio. 2021. FIG. 13 shows the percent survival of the G. mellonella after being treated with the rabbit anti-sera derived from each of the treatment arms. After 72 hours, only approximately 15% of the WT group survived. However, each of the anti-sera arms increased survival to greater than about 90% after 72 hours.

### Example 4: Immunization Study in Rabbits with Exemplary E. faecalis Immunogenic Compositions

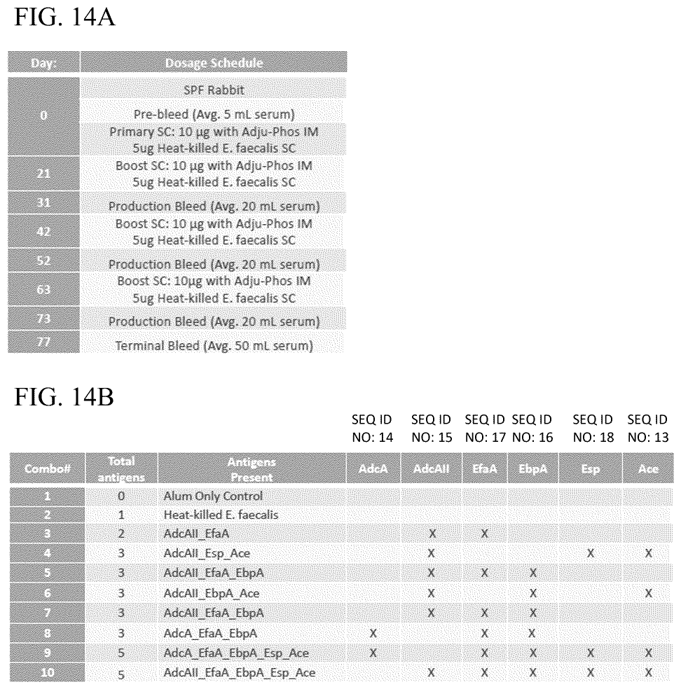

New Zealand White Rabbits were dosed (according to the schedule in FIG. 14A) with an alum control, heat-killed E. faecalis, or one of 8 immunogenic compositions as described herein. The immunogenic compositions tested are shown in FIG. 14B. For each of the immunogenic compositions, the rabbits were dosed at 15 μg per constituent polypeptide antigen.

Antigen-specific IgG ELISAs were used to assess the change in IgG levels post-immunization. FIG. 15 shows that anti-sera derived from animals dosed with individual E. faecalis polypeptide antigens raises antigen-specific IgG titers relative to pre-immunized animals and relative to an alum control.

### Example 5: Immunization Study in Rabbits with Exemplary E. faecalis Immunogenic Compositions

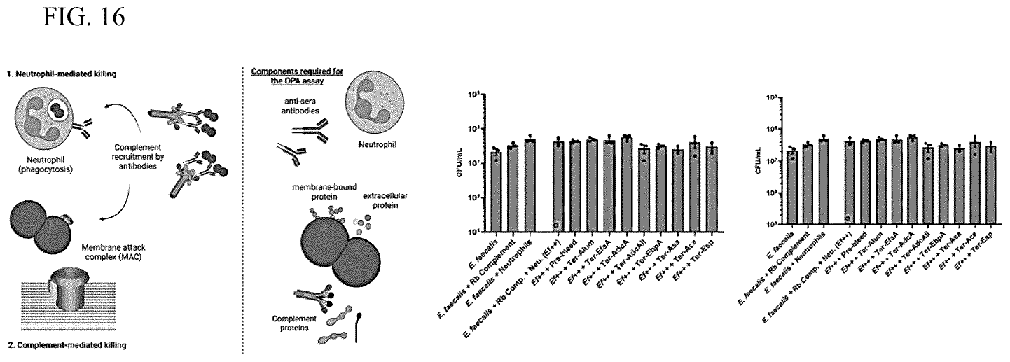

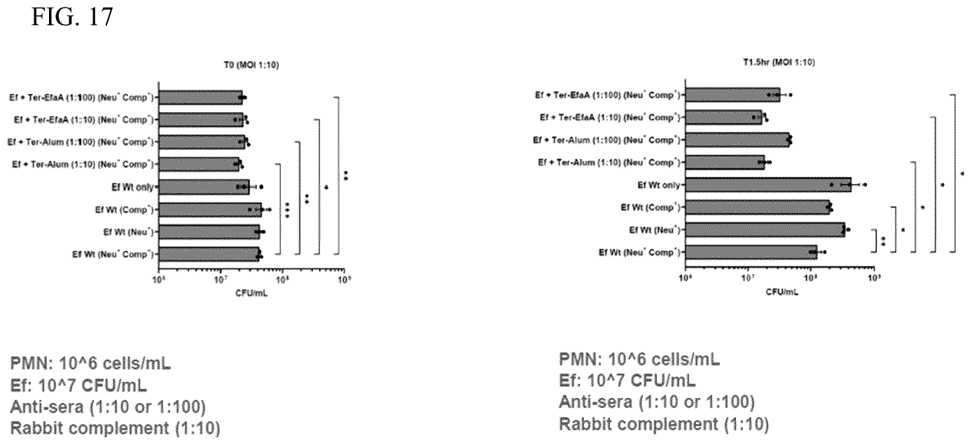

An opsonophagocytic assay (see e.g., Nahm, et. al., 2014, Protocol for multiplexed opsonophagocytic killing assay (UAB-MOPA) for antibodies against Streptococcus pneumoniae) was used to assess antibody-dependent killing efficacy by neutrophils post-immunization. FIG. 16 provides an overview of the opsonophagocytic (OPA) assay. The results at the start of incubation and 90 minutes post-incubation demonstrate that there was opsonophagocytic activity observed when adding the antisera to the bacteria, suggesting that these antigens may be useful as a vaccine antigen. FIG. 17 shows that anti-sera antibodies have an opsonizing/neutralizing effect on neutrophil-mediated killing of E. faecalis in vitro.

### Example 6: Passive and Active Immunization Studies Using a Mouse Model of Peritonitis Infection

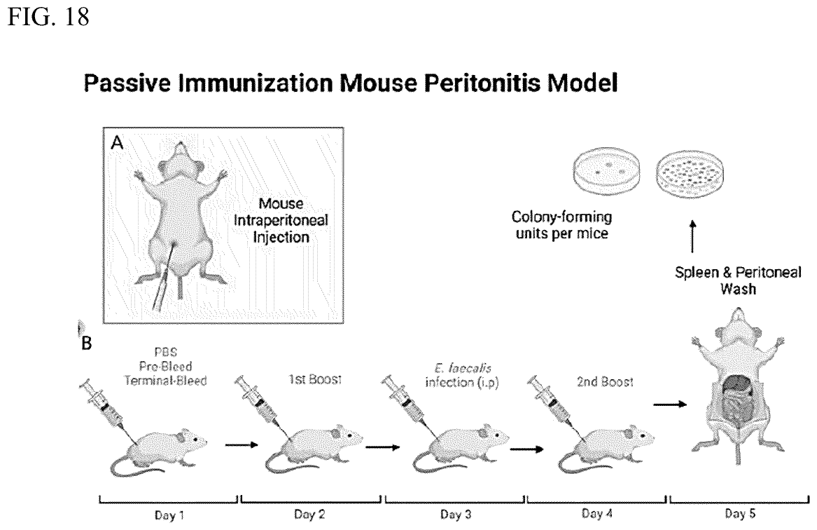

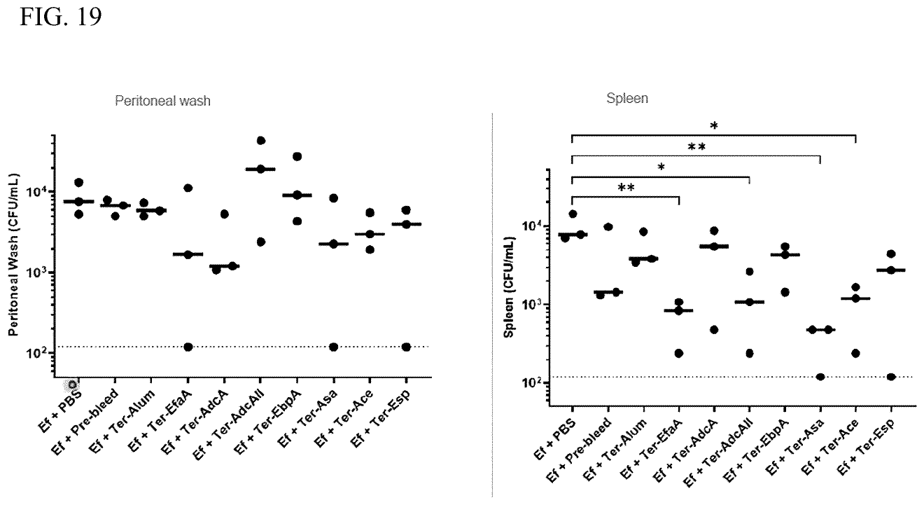

Passive/active immunization studies were conducted by the laboratory of Dr. Jose Lemos at the University of Florida in order to determine the protection efficiency of the anti-sera generated against the E. faecalis antigens. FIG. 18 shows an overview of the mouse peritonitis model used to determine the protection efficacy of the various anti-sera produced against E. faecalis. Briefly, on day 1 a group (N=3 mice per group) of healthy mice (one group for each antigen investigated) undergo primary vaccination with the various mouse antisera generated (3 negative controls of PBS-only, pre-bleed serum and alum-only terminal-bleed sera) as well as seven terminal bleeds from the antigens under study (EfaA, AdcA, AdcAII, EbpA, Asa, Ace and Esp). On day 2, the individual mouse groups receive a boost with the same antisera as day 1. On day 3 the mice are challenged with an injection of E. faecalis OG1RF (158 cells/mL) into the peritoneal cavity. On day 4, the mouse groups receive a second boost vaccination, and on day 5 the mice are sacrificed and the various tissues (spleen and peritoneal cavity) are washed and the wash solution is collected and transferred onto BHI plates to check for colony forming units. The data of FIG. 19 show that relative to the three controls, passive immunization with either anti-EfaA or anti-AdcAII antisera resulted in a significantly decrease (˜5-fold) in E. faecalis CFU recovered from spleen whereas mice immunized with EfaA- or AdcA-specific antisera had reduced bacteria recovered from the peritoneal cavity (1-log and 5-fold, respectively).

### Example 7: Passive Immunization Study Using a Mouse Model of Peritonitis Infection

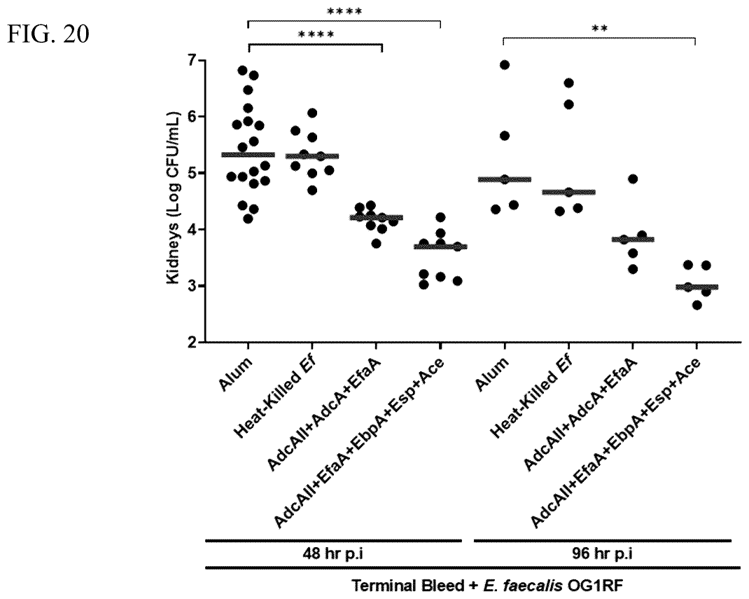

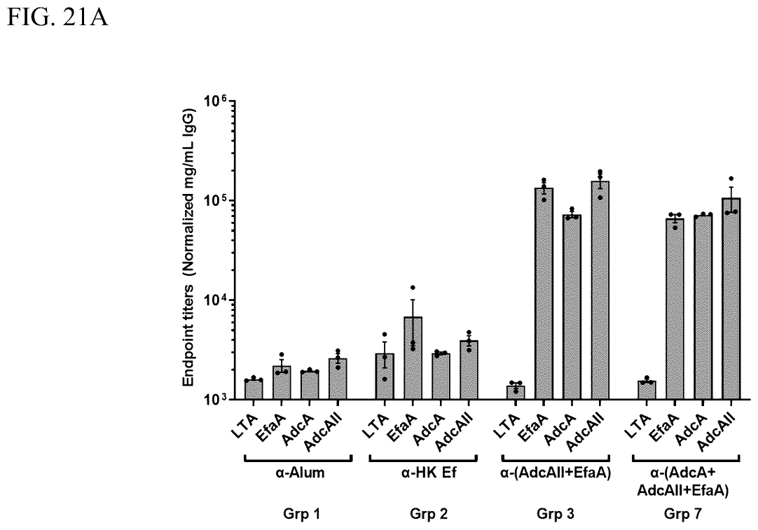

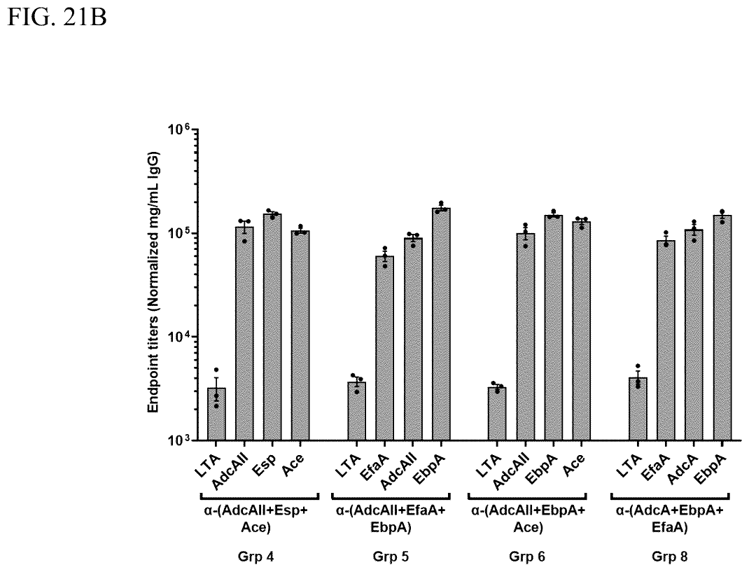

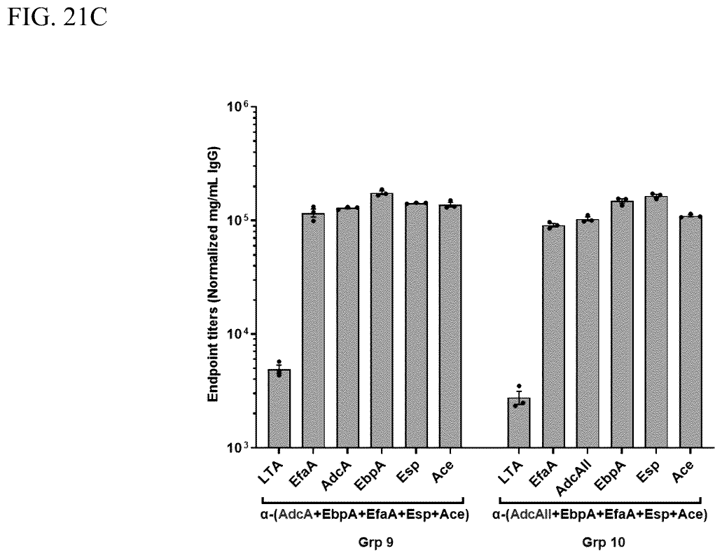

Following the procedure of Example 6, a passive immunization study was conducted in order to determine the protection efficiency of anti-sera generated against E. faecalis antigen combinations. The kidneys of the test subjects were analyzed for colony forming units. FIG. 20 shows the results of this study, where antisera derived from combinations of (AdcAII+AdcA+EfA) and (AdcAII+EfaA+EbpA+Esp+Ace) both significantly decrease E. faecalis CFU recovered from the kidney. Additionally, terminal bleed antigen specific IgG titers were obtained according to the protocols above. FIG. 21A, FIG. 21B, and FIG. 21C show terminal bleed IgG titers for ten treatment groups (Grp 1-Grp 15). All enterococcal peptide combinations show significant IgG titers versus alum control (Grp 1) and heat-killed E. faecalis (Grp 2).

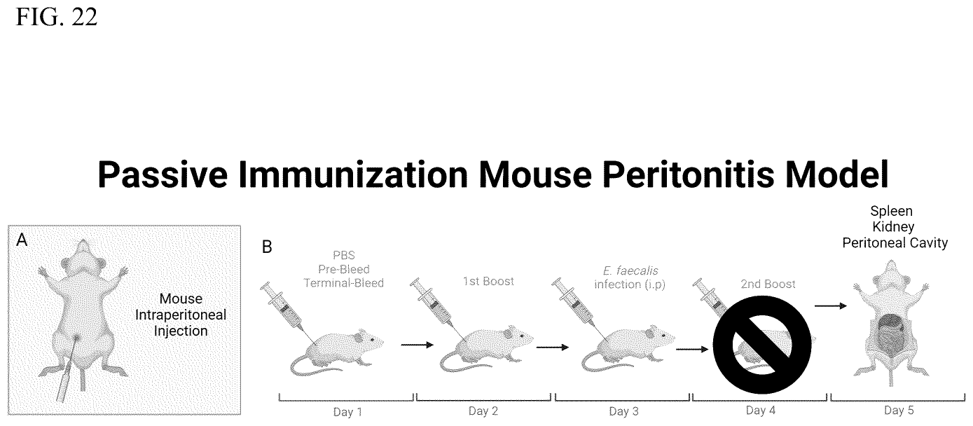

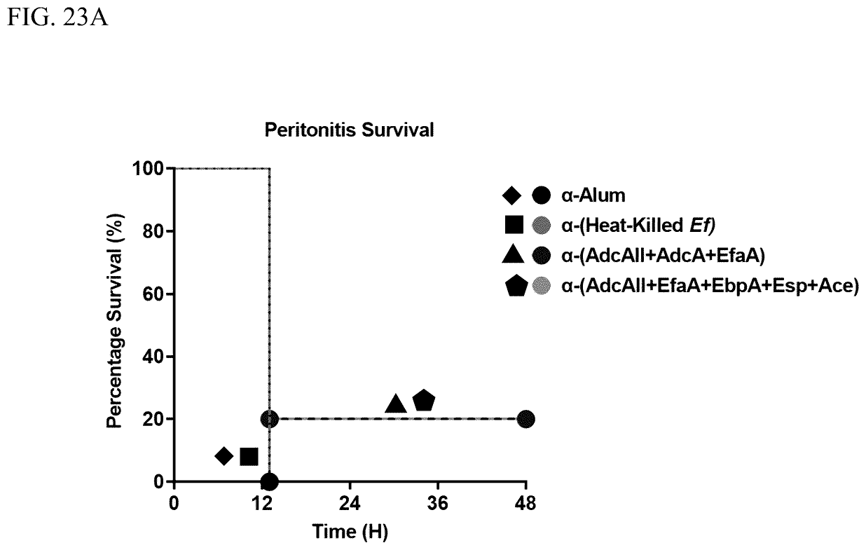

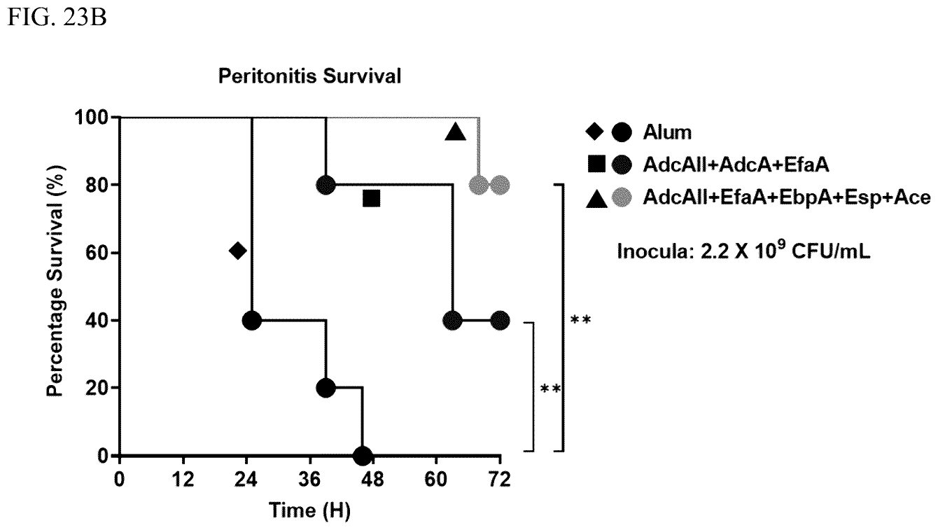

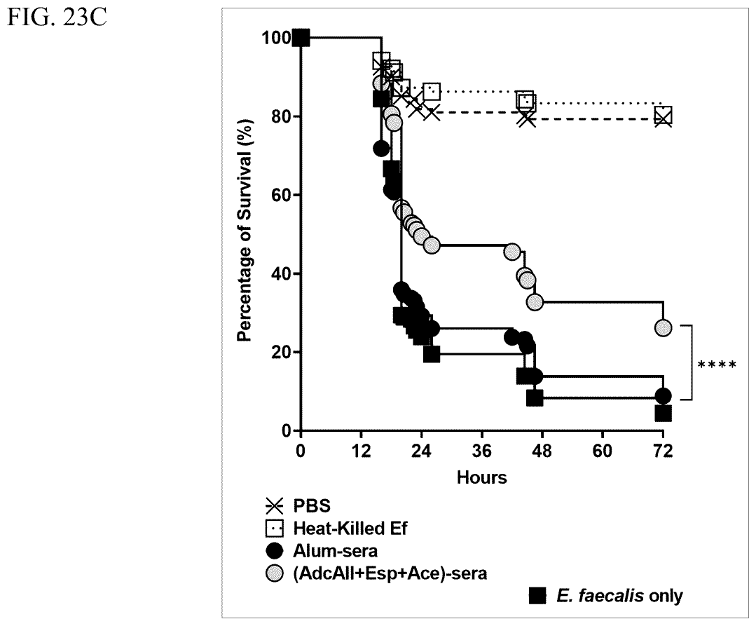

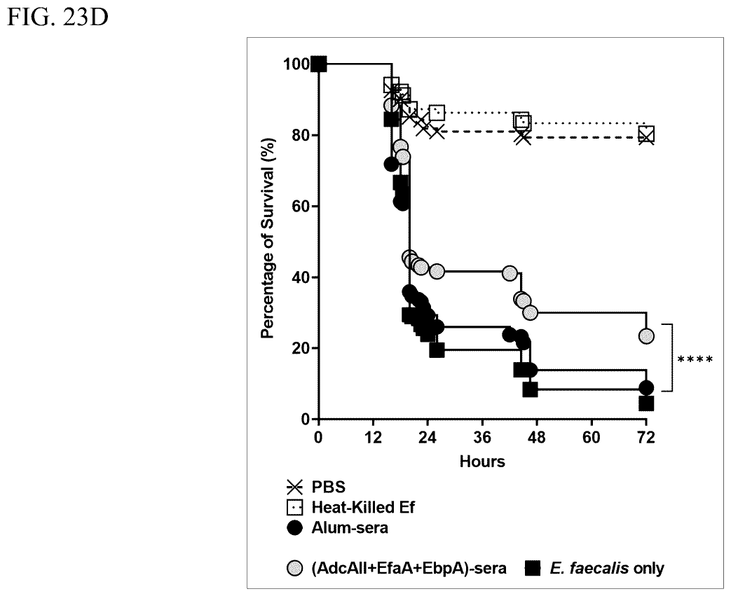

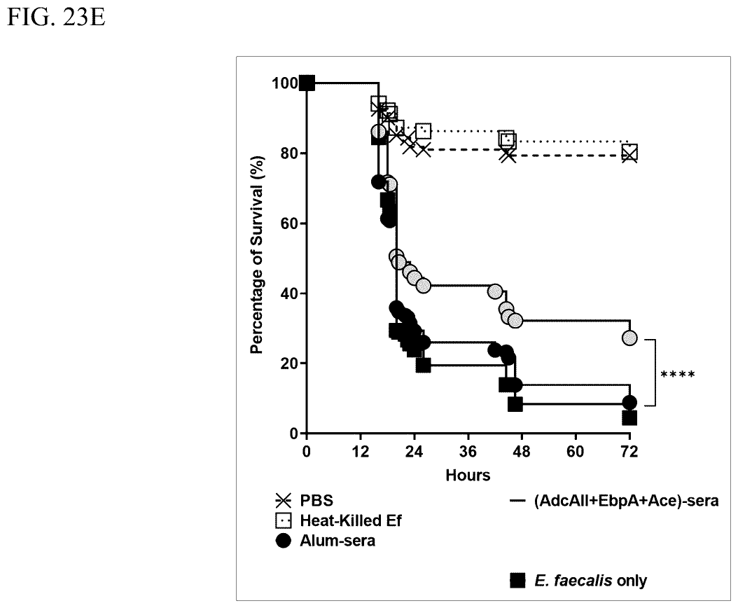

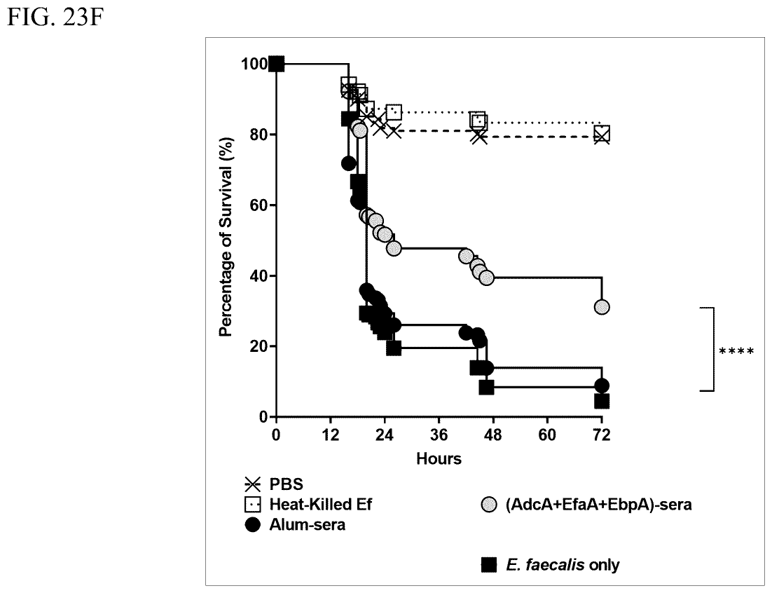

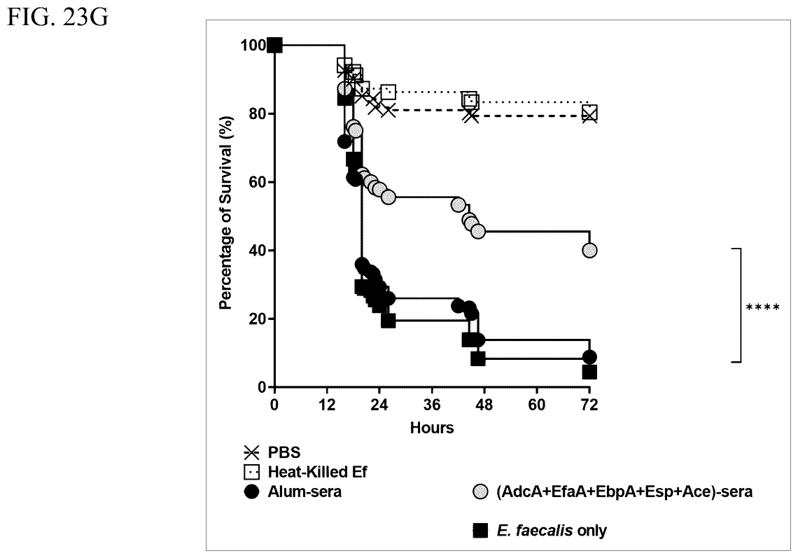

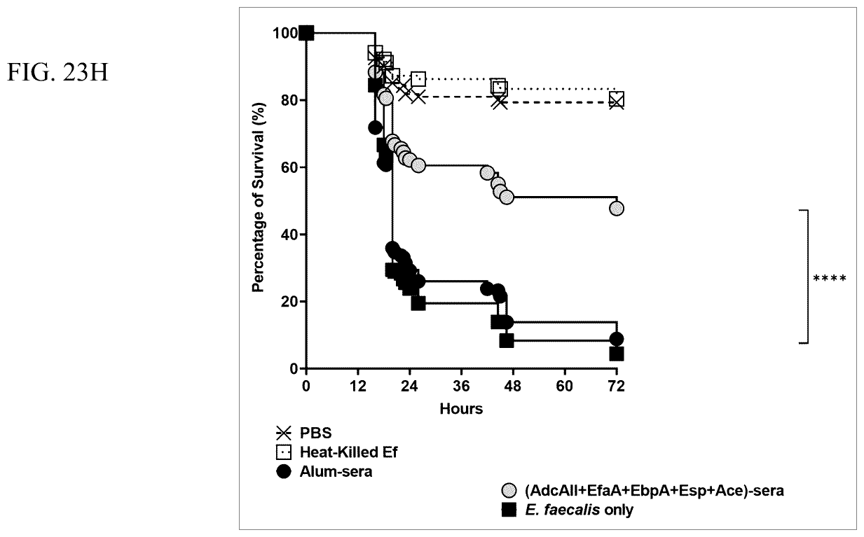

FIG. 22 shows an overview of the mouse peritonitis model used to determine the protection efficacy of the various anti-sera produced against E. faecalis, which is the same procedure described in Example 6, except that no second boost was given on Day 4 of the study. FIG. 23A and FIG. 23B show survival data for animals inoculated with 2.02×15¹⁵ CFU/mL of strain OG1RF and 2.2×15⁹ CFU/mL of strain OG1RF, respectively. In the case of the animals dosed at 2.2×15⁹ CFU/mL, both combinations of (AdcAII+AdcA+EfaA) and (AdcAII+EfA+EbpA+Esp+Ace) resulted in a statistically significant increase in survival at 72 hours post inoculation, versus control.

Survival studies were also conducted with additional antigen combinations (FIGS. 23C, 23D, 23E, 23F, 23G, 23H), with each experimental group demonstrating a statistically significant increase in overall survival of the subjects.

### Example 8: Effectiveness of E. faecalis OG1RF Antigen-Specific Anti-Sera Against Other Clinical Enterococcal Strains

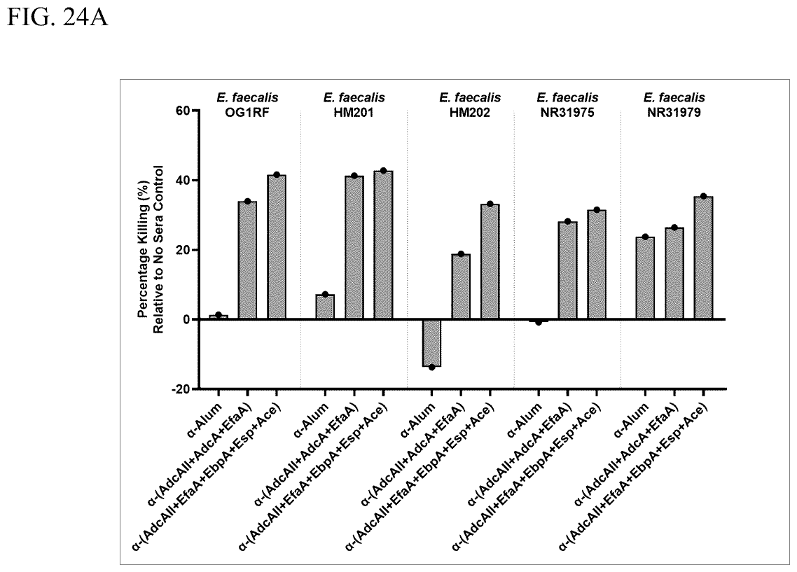

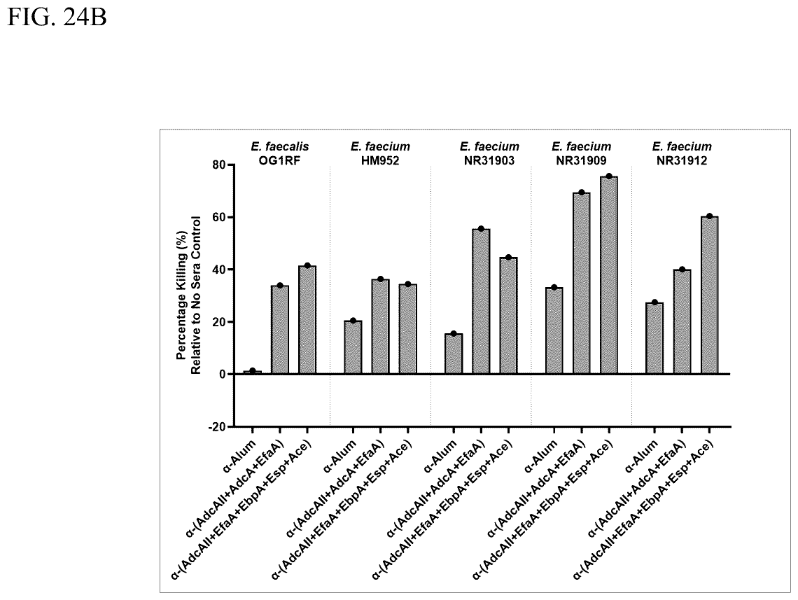

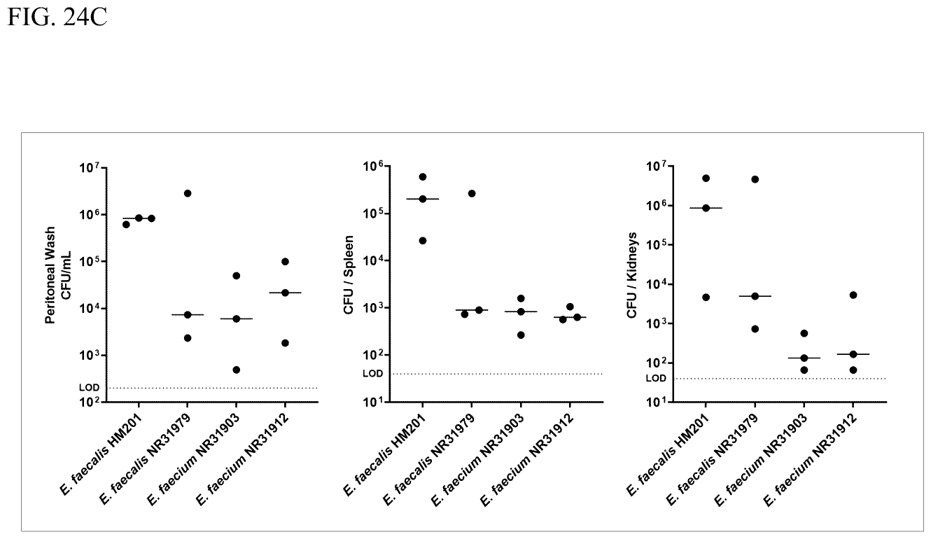

The effectiveness of antigen combinations in other clinically relevant enterococcal strains was determined in the same way described above. FIG. 24A (E. faecalis strains) and FIG. 24B (E. facium) show the results for strains E. faecalis HM201 (blood isolate; VanR), E. faecalis HM202 (fecal isolate; KanR), E. faecalis NR31975 (blood isolate; ErmR GenR), E. faecalis NR31979 (urine isolate; VanR), E. faecium HM952 (normal flora), E. faecium NR31903 (fecal isolate; clonal of CC17 that is AmpR, carry esp, hyl and acm), E. faecium NR31909 (fecal isolate; clonal of CC17), and E. faecium NR31912 (fecal isolate; CC17; VanR) obtained from the NIH BEI Resources Repository. The E. faecium strains exhibit killing efficiency of the tested antigen combinations similar to E. faecalis strains, showing that the combinations described herein are capable of treating infections of multiple Enterococcal species and strains thereof.

FIG. 24C shows a summary of colony forming unit (CFU) data generated from a mouse peritoneal (peritonitis) infection model (as described previously) for E. faecalis HM201, E. faecalis NR31979, E. faecium NR31903, and E. faecium NR31912.

What is claimed is:

1. An immunogenic composition comprising at least one recombinant Enterococcus faecalis (E. faecalis) polypeptide antigen or a fragment thereof.

2. The immunogenic composition of claim 1, wherein the at least one recombinant E. faecalis polypeptide antigen is an Ace polypeptide antigen, an AdcA polypeptide antigen, an AdcAII polypeptide antigen, an EbpA polypeptide antigen, an EfaA polypeptide antigen, or an Esp polypeptide antigen.

3. The immunogenic composition of claim 1 or 2, wherein the at least one recombinant E. faecalis polypeptide antigen comprises a first recombinant E. faecalis polypeptide antigen and a second recombinant E. faecalis polypeptide antigen.

4. The immunogenic composition of claim 3, wherein the first recombinant E. faecalis polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

5. The immunogenic composition of any one of claims 1-4, wherein the at least one recombinant E. faecalis polypeptide antigen or the first recombinant E. faecalis polypeptide antigen is an Ace polypeptide antigen.

6. The immunogenic composition of any one of claims 1-4, wherein the at least one recombinant E. faecalis polypeptide antigen or the first recombinant E. faecalis polypeptide antigen is an AdcA polypeptide antigen.

7. The immunogenic composition of any one of claims 1-4, wherein the at least one recombinant E. faecalis polypeptide antigen or the first recombinant E. faecalis polypeptide antigen is an AdcAII polypeptide antigen.

8. The immunogenic composition of any one of claims 1-4, wherein the at least one recombinant E. faecalis polypeptide antigen or the first recombinant E. faecalis polypeptide antigen is an Ebp polypeptide antigen.

9. The immunogenic composition of any one of claims 1-4, wherein the at least one recombinant E. faecalis polypeptide antigen or the first recombinant E. faecalis polypeptide antigen is an EfaA polypeptide antigen.

10. The immunogenic composition of any one of claims 1-4, wherein the at least one recombinant E. faecalis polypeptide antigen or the first recombinant E. faecalis polypeptide antigen is an Esp polypeptide antigen.

11. The immunogenic composition of any one of claims 3-10, wherein the at least one recombinant E. faecalis polypeptide antigen comprises a second recombinant E. faecalis polypeptide antigen, wherein the second recombinant E. faecalis polypeptide antigen is an Ace polypeptide antigen, an AdcA polypeptide antigen, an AdcAII polypeptide antigen, an Ebp polypeptide antigen, an EfaA polypeptide antigen, or an Esp polypeptide antigen.

12. The immunogenic composition of any one of claims 3-11, wherein the at least one recombinant E. faecalis polypeptide antigen comprises a third recombinant E. faecalis polypeptide antigen, wherein the third recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

13. The immunogenic composition of any one of claims 3-12, wherein the at least one recombinant E. faecalis polypeptide antigen comprises a fourth E. faecalis polypeptide antigen, wherein the fourth E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

14. The immunogenic composition of any one of claims 3-13, wherein the at least one recombinant E. faecalis polypeptide antigen comprises a fifth recombinant E. faecalis polypeptide antigen, wherein the fifth recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

15. The immunogenic composition of any one of claims 3-14, wherein the at least one recombinant E. faecalis polypeptide antigen comprises a sixth recombinant E. faecalis polypeptide antigen, wherein the sixth recombinant E. faecalis polypeptide antigen is: an Ace polypeptide antigen, an AdcA polypeptide antigen, an AdcAII polypeptide antigen, an Ebp polypeptide antigen, an EfaA polypeptide antigen, or an Esp polypeptide antigen.

16. The immunogenic composition of any one of claims 2-15, wherein:
the Ace polypeptide antigen comprises or consists of the amino acid sequence of SEQ ID NO: 1 or SEQ ID NO: 13;
the AdcA polypeptide antigen comprises or consists of the amino acid sequence of SEQ ID NO: 2 or SEQ ID NO: 14;
the AdcAII polypeptide antigen comprises or consists of the amino acid sequence of SEQ ID NO: 3 or SEQ ID NO: 15;
the EbpA polypeptide antigen comprises or consists of the amino acid sequence of SEQ ID NO: 4 or SEQ ID NO: 16;
the EfaA polypeptide antigen comprises or consists of the amino acid sequence of SEQ ID NO: 5 or SEQ ID NO: 17; and
the Esp polypeptide antigen comprises or consists of the amino acid sequence of SEQ ID NO: 6 or SEQ ID NO: 18.

17. The immunogenic composition of any one of claims 1-16, wherein the at least one recombinant E. faecalis polypeptide antigen comprises or consists of between 3 and 5 recombinant E. faecalis polypeptide antigen.

18. The immunogenic composition of any one of claims 3-17, wherein the first recombinant E. faecalis polypeptide antigen is an AdcAII polypeptide antigen and the second recombinant E. faecalis polypeptide antigen is an EfaA polypeptide antigen.

19. The immunogenic composition of any one of claims 13-17, wherein the first recombinant E. faecalis polypeptide antigen is an AdcAII polypeptide antigen, the second recombinant E. faecalis polypeptide antigen is an Esp polypeptide antigen, and the third recombinant E. faecalis polypeptide antigen is an Ace polypeptide antigen.

20. The immunogenic composition of any one of claims 13-17, wherein the first recombinant E. faecalis polypeptide antigen is an AdcAII polypeptide antigen, the second recombinant E. faecalis polypeptide antigen is an EfaA polypeptide antigen, and the third recombinant E. faecalis polypeptide antigen is an EbpA polypeptide antigen.

21. The immunogenic composition of any one of claims 13-17, wherein the first recombinant E. faecalis polypeptide antigen is an AdcAII polypeptide antigen, the second recombinant E. faecalis polypeptide antigen is an Ace polypeptide antigen, and the third recombinant E. faecalis polypeptide antigen is an EbpA polypeptide antigen.

22. The immunogenic composition of any one of claims 13-17, wherein the first recombinant E. faecalis polypeptide antigen is an AdcA polypeptide antigen, the second recombinant E. faecalis polypeptide antigen is an EfaA polypeptide antigen, and the third recombinant E. faecalis polypeptide antigen is an EbpA polypeptide antigen.

23. The immunogenic composition of any one of claims 15-17, wherein the first recombinant E. faecalis polypeptide antigen is an AdcA polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen, and the fifth recombinant E. faecalis polypeptide antigen is an Ace polypeptide antigen.

24. The immunogenic composition of any one of claims 14-17, wherein the first recombinant E. faecalis polypeptide antigen is an AdcAII polypeptide antigen, the second recombinant E. faecalis polypeptide antigen is an EfaA polypeptide antigen, the third recombinant E. faecalis polypeptide antigen is an EbpA polypeptide antigen, the fourth recombinant E. faecalis polypeptide antigen is an Esp polypeptide antigen; and the fifth recombinant E. faecalis polypeptide antigen is an Ace polypeptide antigen.

25. The immunogenic composition of any one of claim 1, 2, 4-15, or 16, wherein the composition does not comprise a second recombinant E. faecalis polypeptide antigen.

26. The immunogenic composition of any one of claim 3-11 or 16, wherein the composition does not comprise a third recombinant E. faecalis polypeptide antigen.

27. The immunogenic composition of any one of claim 3-12 or 16-22, wherein the composition does not comprise a fourth recombinant E. faecalis polypeptide antigen.

28. The immunogenic composition of any one of claim 3-13 or 16-22, wherein the composition does not comprise a fifth recombinant E. faecalis polypeptide antigen.

29. The immunogenic composition of any one of claim 3-14 or 16-24, wherein the composition does not comprise a sixth recombinant E. faecalis polypeptide antigen.

30. The immunogenic composition of any one of claim 2-24 or 26-29, wherein the AdcAII polypeptide antigen comprises an amino acid sequence that is at least 85%, at least 90%, or at least 95% identical to SEQ ID NO: 3 or SEQ ID NO: 15; and the EfaA polypeptide antigen comprises an amino acid sequence that is at least 95% identical to SEQ ID NO: 5 or SEQ ID NO: 17.

31. The immunogenic composition of any one of claim 2-24 or 26-29, wherein the AdcAII polypeptide antigen comprises or consists of the amino acid sequence of SEQ ID NO: 3 or SEQ ID NO: 15; and the EfaA polypeptide antigen comprises or consists of the amino acid sequence of SEQ ID NO: 5 or SEQ ID NO: 17.

32. The immunogenic composition of any one of claim 2-24 or 27-29, wherein the AdcAII polypeptide antigen comprises an amino acid sequence that is at least 85%, at least 90%, or at least 95% identical to SEQ ID NO: 3 or SEQ ID NO: 15; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

33. The immunogenic composition of any one of claim 2-24 or 27-29, wherein the AdcAII polypeptide antigen comprises or consists of the amino acid sequence of SEQ ID NO: 3 or SEQ ID NO: 15; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

34. The immunogenic composition of any one of claim 2-24 or 27-29, wherein the AdcAII polypeptide antigen comprises an amino acid sequence that is at least 85%, at least 90%, or at least 95% identical to SEQ ID NO: 3 or SEQ ID NO: 15; the EfaA polypeptide antigen comprises an amino acid sequence that is at least 95% identical to SEQ ID NO: 5 or SEQ ID NO: 17; and the EbpA polypeptide antigen comprises an amino acid sequence that is at least 95% identical to SEQ ID NO: 4 or SEQ ID NO: 16.

35. The immunogenic composition of any one of claim 2-24 or 27-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

36. The immunogenic composition of any one of claim 2-24 or 27-29, wherein the AdcAII polypeptide antigen comprises an amino acid sequence that is at least 85%, at least 90%, or at least 95% identical to SEQ ID NO: 3 or SEQ ID NO: 15; the Ace polypeptide antigen comprises an amino acid sequence that is at least 95% identical to SEQ ID NO: 1 or SEQ ID NO: 13; and the EbpA polypeptide antigen comprises an amino acid sequence that is at least 95% identical to SEQ ID NO: 4 or SEQ ID NO: 16.

37. The immunogenic composition of any one of claim 2-24 or 27-29, wherein the AdcAII polypeptide antigen comprises or consists of the amino acid sequence of SEQ ID NO: 3 or SEQ ID NO: 15; the Ace polypeptide antigen comprises or consists of the amino acid sequence of SEQ ID NO: 1 or SEQ ID NO: 13; and the EbpA polypeptide antigen comprises or consists of the amino acid sequence of SEQ ID NO: 4 or SEQ ID NO: 16.

38. The immunogenic composition of any one of claim 2-24 or 27-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

39. The immunogenic composition of any one of claim 2-24 or 27-29, wherein the AdcA polypeptide antigen comprises an amino acid sequence that is at least 85%, at least 90%, or at least 95% identical to SEQ ID NO: 2 or SEQ ID NO: 14; the EfaA polypeptide antigen comprises an amino acid sequence that is at least 95% identical to SEQ ID NO: 5 or SEQ ID NO: 17; and the EbpA polypeptide antigen comprises an amino acid sequence that is at least 95% identical to SEQ ID NO: 4 or SEQ ID NO: 16.

40. The immunogenic composition of any one of claim 2-24 or 27-29, wherein the AdcA polypeptide antigen comprises or consists of the amino acid sequence of SEQ ID NO: 2 or SEQ ID NO: 14; the EfaA polypeptide antigen comprises or consists of the amino acid sequence of SEQ ID NO: 5 or SEQ ID NO: 17; and the EbpA polypeptide antigen comprises or consists of the amino acid sequence of SEQ ID NO: 4 or SEQ ID NO: 16.

41. The immunogenic composition of any one of claim 2-24 or 28-29, wherein the AdcA polypeptide antigen comprises an amino acid sequence that is at least 85%, at least 90%, or at least 95% identical to SEQ ID NO: 2 or SEQ ID NO: 14; the EfaA polypeptide antigen comprises an amino acid sequence that is at least 95% identical to SEQ ID NO: 5 or SEQ ID NO: 17; the EbpA polypeptide antigen comprises an amino acid sequence that is at least 95% identical to SEQ ID NO: 4 or SEQ ID NO: 16; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

42. The immunogenic composition of any one of claim 2-24 or 28-29, wherein the AdcA polypeptide antigen comprises or consists of the amino acid sequence of SEQ ID NO: 2 or SEQ ID NO: 14; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

43. The immunogenic composition of any one of claim 2-24 or 28-29, wherein the AdcAII polypeptide antigen comprises an amino acid sequence that is at least 85%, at least 90%, or at least 95% identical to SEQ ID NO: 3 or SEQ ID NO: 15; the EfaA polypeptide antigen comprises an amino acid sequence that is at least 95% identical to SEQ ID NO: 5 or SEQ ID NO: 17; the EbpA polypeptide antigen comprises an amino acid sequence that is at least 95% identical to SEQ ID NO: 4 or SEQ ID NO: 16; the Esp polypeptide antigen comprises an amino acid sequence that is at least 95% identical to SEQ ID NO: 6 or SEQ ID NO: 18; and the Ace polypeptide antigen comprises an amino acid sequence that is at least 95% identical to SEQ ID NO: 1 or SEQ ID NO: 13.

44. The immunogenic composition of any one of claim 2-24 or 28-29, wherein the AdcAII polypeptide antigen comprises or consists of the amino acid sequence of SEQ ID NO: 3 or SEQ ID NO: 15; the EfaA polypeptide antigen comprises or consists of the amino acid sequence of SEQ ID NO: 5 or SEQ ID NO: 17; the EbpA polypeptide antigen comprises or consists of the amino acid sequence of SEQ ID NO: 4 or SEQ ID NO: 16; the Esp polypeptide antigen comprises or consists of the amino acid sequence of SEQ ID NO: 6 or SEQ ID NO: 18; and the Ace polypeptide antigen comprises or consists of the amino acid sequence of SEQ ID NO: 1 or SEQ ID NO: 13.

45. The immunogenic composition of any one of claims 1-44, further comprising one or more adjuvants selected from alum, saponin, monophosphoryl lipid A (MPLA), TLR-agonist, or combinations thereof.

46. The immunogenic composition of any one of claims 1-45, further comprising at least one excipient.

47. The immunogenic composition of claim 46, wherein the at least one excipient is selected from vehicles, solubilizers, emulsifiers, stabilizers, preservatives, isotonicity agents, buffer systems, dispersants, diluents, viscosity modifiers, and absorption enhancers.

48. The immunogenic composition of any one of claims 45-47, wherein the composition is formulated as a sterile injectable solution.

49. A method of inducing a protective immune response against an Enterococcus bacterium in a subject comprising administering the immunogenic composition of any one of claims 1-48 to the subject.

50. The method of claim 49, wherein the immunogenic composition induces an antibody response in the subject against the Enterococcus bacterium and does not induce an antibody response in the subject against human tissue.

51. The immunogenic composition of any one of claims 1-48 for use in treating a disorder in a subject in need thereof.

52. The immunogenic composition of any one of claims 1-48 for use in treating a disorder in a subject in need thereof, wherein the disorder is related to infection by an Enterococcus bacterium.

53. Use of the immunogenic composition of any one of claims 1-48 for inducing a protective immune response against an Enterococcus bacterium in a subject.

54. Use of the immunogenic composition of any one of claims 1-44 in the manufacture of a medicament for inducing a protective immune response against an Enterococcus bacterium in a subject.

55. The method of claim 49 or 50, the immunogenic composition for use of claim 51 or 52, or the use of claim 53 or 54, wherein the Enterococcus bacterium is E. faecalis, E. faecium, or E. durans.

56. The method of any one of claim 49 or 50, the immunogenic composition for use of claim 51 or 52, or the use of claim 53 or 54, wherein the Enterococcus bacterium is E. faecalis.

57. The method of claims 49-50, the immunogenic composition for use of claim 51 or 52, or the use of claim 53 or 54, wherein the subject is at risk for, or is experiencing, root canal failure, endocarditis, bacteremia, urinary tract infections, prostatitis, intraabdominal infection, cellulitis, dysbiotic gastrointestinal tract, prosthetic joint infection, or wound infections.

58. A method of any one of claim 49 or 50, the immunogenic composition for use of claim 51 or 52, or the use of claim 53 or 54, wherein the risk of root canal failure, endocarditis, bacteremia, urinary tract infections, prostatitis, intraabdominal infection, cellulitis, dysbiotic gastrointestinal tract, prosthetic joint infection, or wound infections in a subject is reduced compared to before administration of the immunogenic composition.

59. The immunogenic composition of any one of claims 1-48, the method of any one of claims 49-50, 55-58, the immunogenic composition for use of claim 51 or 52, or the use of claim 53 or 54, wherein one or more of the at least one polypeptide antigen is a fragment of the polypeptide antigen.

60. The immunogenic composition, method, immunogenic composition for use, or use of claim 59, wherein the fragment of the at least one polypeptide antigen is at least 15%, 15%, 20%, 25%, 30%, 35%, 40%, 45%, 50%, 55%, 60%, 65%, 70%, 75%, 80%, 85%, 90%, or 95% of the length of the at least one polypeptide antigen.

61. Invention of product, process, system, kit or use, characterized by one or more elements disclosed in the application.
